(19) United States
(12) Patent Application Publication
(10) Pub. No.: US 2026/0021045 A1
(43) Pub. Date: Jan. 22, 2026

(54) METHODS AND FORMULATIONS FOR TREATMENT OF LIVER CANCER USING TRIFLURIDINE

(71) Applicant: JND Therapeutics, Inc., Wellesley, MA (US)

(72) Inventors: David Dove, Tucson, AZ (US); Jonathan Reis, Wellesley, MA (US); Yair Fisher, Avigdor (IL); Roger Zanon, Minnetonka, MN (US)

(73) Assignee: JND Therapeutics, Inc., Wellesley, MA (US)

(21) Appl. No.: 19/270,727

(22) Filed: Jul. 16, 2025

Related U.S. Application Data
(60) Provisional application No. 63/672,594, filed on Jul. 17, 2024.
(60) Provisional application No. 63/758,622, filed on Feb. 14, 2025.

(51) Int. Cl. A61K 9/08; A61K 9/00; A61K 31/7072; A61K 47/12; A61K 47/18; A61P 35/00

(52) U.S. Cl. CPC A61K 9/08; A61K 9/0019; A61K 31/7072; A61K 47/12; A61K 47/18; A61P 35/00

(57) ABSTRACT

The present disclosure provides methods of treating liver cancer using trifluridine. The methods include administering to a subject a therapeutically effective amount of trifluridine by hepatic arterial infusion. The methods include administering to a subject a therapeutically effective amount of trifluridine by hepatic arterial infusion, wherein the subject's alanine aminotransferase, aspartate aminotransferase, alkaline phosphatase, and bilirubin levels do not rise more than about 20% above the subject's alanine aminotransferase, aspartate aminotransferase, alkaline phosphatase, and bilirubin levels prior to administration of the therapeutically effective amount of trifluridine. The present disclosure provides a liquid pharmaceutical formulation comprising trifluridine, a buffer, and a solvent system comprising water and a co-solvent, wherein the formulation has a pH of about 1 to 8. The present disclosure also provides a method of treating liver cancer with the liquid pharmaceutical formulation.

### CROSS-REFERENCE TO RELATED APPLICATIONS

This application claims the benefit of U.S. Provisional Patent Application Ser. No. 63/672,594, filed on Jul. 17, 2024, entitled “METHODS OF TREATMENT OF LIVER CANCER USING TRIFLURIDINE,” and U.S. Provisional Patent Application Ser. No. 63/758,622, filed on Feb. 14, 2025, entitled “TRIFLURIDINE FORMULATIONS AND METHODS OF TREATMENT OF LIVER CANCER USING TRIFLURIDINE,” the disclosures of which are incorporated herein by reference in their entireties.

### FIELD OF THE INVENTION

This invention relates to methods of treating liver cancer by administration of a therapeutically effective amount of trifluridine (FTD) by local administration or by hepatic arterial infusion (HAI). This invention further relates to pharmaceutical formulations that contain trifluridine.

### BACKGROUND OF THE INVENTION

Primary liver cancer is one of the most common forms of cancer in the world and the third leading cause of cancer deaths worldwide. Secondary liver cancer, or metastasis in the liver, is a cancer that starts somewhere else in the body and then spreads to the liver. Examples of secondary liver cancer include many common types of cancer, such as cancers of the colon, rectum, lung, and pancreas, melanoma, including ocular melanoma, and breast cancer. Treatment options for liver cancer have been limited, especially for late stage liver cancer patients. Surgery and radiation therapy are options for early stage liver cancer, but not very effective for advanced liver cancer. Systemic chemotherapies have not been particularly effective, and there are a limited number of drugs available for use.

Currently available treatment options can present extremely high levels of toxicity to liver cancer patients. For example, one treatment used to treat metastatic liver cancer, floxuridine, is a highly toxic drug with a narrow margin of safety. In patients with a high number of metastases, floxuridine is delivered via hepatic artery infusion in order to deliver a targeted higher dose. This route of administration has been associated with resolving hepatocyte necrosis but also with permanent cholangiocyte injury, progressing to biliary sclerosis, portal fibrosis, and permanent biliary obstruction, likely due to drug toxicity. Patients treated with floxuridine are carefully supervised since therapeutic response is unlikely to occur without some evidence of toxicity. Severe toxicity, gastrointestinal hemorrhage and even death may result from the use of floxuridine despite meticulous selection of patients and careful adjustment of dosage. Although severe toxicity is more likely in poor risk patients, fatalities may be encountered occasionally, even in patients in relatively good condition. Accordingly, there is a need for new effective treatments of liver cancer, both primary and metastatic.

### SUMMARY OF THE INVENTION

The present disclosure provides methods of treatment of liver cancer in a subject in need thereof by administration of a therapeutically effective amount of trifluridine (FTD) by local administration. The present disclosure also provides methods of treatment of liver cancer in a subject in need thereof by administration of a therapeutically effective amount of trifluridine by intravenous or intra-arterial delivery. Trifluridine is an antineoplastic thymidine-based nucleoside analogue and is described chemically as 2′-deoxy-5-(trifluoromethyl)uridine or α,α,α-trifluorothymidine. Its chemical structure is shown below in Formula I.

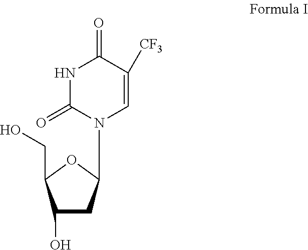 [SMILES: O=c1[nH]c(=O)n([C@H]2C[C@@H](O)[C@@H](CO)O2)cc1C(F)(F)F]

In some aspects, the subject to be treated is a human.

In some aspects, the subject to be treated is an human adult.

In some aspects, the subject to be treated is a human child.

In some aspects, the therapeutically effective amount of trifluridine is administered in the form of a formulation as described in more detail below.

In some aspects, the therapeutically effective amount of trifluridine is continuously administered by hepatic arterial infusion.

In some aspects, the therapeutically effective amount of trifluridine is continuously administered by hepatic arterial infusion via a pump.

In some aspects, the therapeutically effective amount of trifluridine is about 0.01 mg/kg/day to about 3 mg/kg/day.

In some aspects, the therapeutically effective amount of trifluridine is about 0.1 mg/kg/day to about 2 mg/kg/day.

In some aspects, the therapeutically effective amount of trifluridine is about 0.7 mg/kg/day to about 1 mg/kg/day.

In some aspects, the therapeutically effective amount of trifluridine is continuously administered for about 30 minutes to about 1 month.

In some aspects, the therapeutically effective amount of trifluridine is continuously administered for at least 7 days.

In some aspects, the therapeutically effective amount of trifluridine is continuously administered for at least 14 days.

In some aspects, the therapeutically effective amount of trifluridine is continuously administered for at least 28 days.

In some aspects, after the therapeutically effective amount of trifluridine is administered for at least 7 days, the subject's alanine aminotransferase level does not rise more than about 20% above the subject's alanine aminotransferase level prior to administration of the therapeutically effective amount of trifluridine.

In some aspects, after the therapeutically effective amount of trifluridine is administered for at least 14 days, the subject's alanine aminotransferase level does not rise more than about 20% above the subject's alanine aminotransferase level prior to administration of the therapeutically effective amount of trifluridine.

In some aspects, after the therapeutically effective amount of trifluridine is administered for at least 28 days, the subject's alanine aminotransferase level does not rise more than about 20% above the subject's alanine aminotransferase level prior to administration of the therapeutically effective amount of trifluridine.

In some aspects, after the therapeutically effective amount of trifluridine is administered for at least 7 days, the subject's alanine aminotransferase level does not rise more than about 20 U/L above the subject's alanine aminotransferase level prior to administration of the therapeutically effective amount of trifluridine.

In some aspects, after the therapeutically effective amount of trifluridine is administered for at least 14 days, the subject's alanine aminotransferase level does not rise more than about 20 U/L above the subject's alanine aminotransferase level prior to administration of the therapeutically effective amount of trifluridine.

In some aspects, after the therapeutically effective amount of trifluridine is administered for at least 28 days, the subject's alanine aminotransferase level does not rise more than about 20 U/L above the subject's alanine aminotransferase level prior to administration of the therapeutically effective amount of trifluridine.

In some aspects, after the therapeutically effective amount of trifluridine is administered for at least 7 days, the subject's aspartate aminotransferase level does not rise more than about 20% above the subject's aspartate aminotransferase level prior to administration of the therapeutically effective amount of trifluridine.

In some aspects, after the therapeutically effective amount of trifluridine is administered for at least 14 days, the subject's aspartate aminotransferase level does not rise more than about 20% above the subject's aspartate aminotransferase level prior to administration of the therapeutically effective amount of trifluridine.

In some aspects, after the therapeutically effective amount of trifluridine is administered for at least 28 days, the subject's aspartate aminotransferase level does not rise more than about 20% above the subject's aspartate aminotransferase level prior to administration of the therapeutically effective amount of trifluridine.

In some aspects, after the therapeutically effective amount of trifluridine is administered for at least 7 days, the subject's aspartate aminotransferase level does not rise more than about 20 U/L above the subject's aspartate aminotransferase level prior to administration of the therapeutically effective amount of trifluridine.

In some aspects, after the therapeutically effective amount of trifluridine is administered for at least 14 days, the subject's aspartate aminotransferase level does not rise more than about 20 U/L above the subject's aspartate aminotransferase level prior to administration of the therapeutically effective amount of trifluridine.

In some aspects, after the therapeutically effective amount of trifluridine is administered for at least 28 days, the subject's aspartate aminotransferase level does not rise more than about 20 U/L above the subject's aspartate aminotransferase level prior to administration of the therapeutically effective amount of trifluridine.

In some aspects, after the therapeutically effective amount of trifluridine is administered for at least 7 days, the subject's alkaline phosphatase level does not rise more than about 20% above the subject's alkaline phosphatase level prior to administration of the therapeutically effective amount of trifluridine.

In some aspects, after the therapeutically effective amount of trifluridine is administered for at least 14 days, the subject's alkaline phosphatase level does not rise more than about 20% above the subject's alkaline phosphatase level prior to administration of the therapeutically effective amount of trifluridine.

In some aspects, after the therapeutically effective amount of trifluridine is administered for at least 28 days, the subject's alkaline phosphatase level does not rise more than about 20% above the subject's alkaline phosphatase level prior to administration of the therapeutically effective amount of trifluridine.

In some aspects, after the therapeutically effective amount of trifluridine is administered for at least 7 days, the subject's alkaline phosphatase level does not rise more than about 50 IU/L above the subject's alkaline phosphatase level prior to administration of the therapeutically effective amount of trifluridine.

In some aspects, after the therapeutically effective amount of trifluridine is administered for at least 14 days, the subject's alkaline phosphatase level does not rise more than about 50 IU/L above the subject's alkaline phosphatase level prior to administration of the therapeutically effective amount of trifluridine.

In some aspects, after the therapeutically effective amount of trifluridine is administered for at least 28 days, the subject's alkaline phosphatase level does not rise more than about 50 IU/L above the subject's alkaline phosphatase level prior to administration of the therapeutically effective amount of trifluridine.

In some aspects, after the therapeutically effective amount of trifluridine is administered for at least 7 days, the subject's total bilirubin level does not rise more than about 20% above the subject's total bilirubin level prior to administration of the therapeutically effective amount of trifluridine.

In some aspects, after the therapeutically effective amount of trifluridine is administered for at least 14 days, the subject's total bilirubin level does not rise more than about 20% above the subject's total bilirubin level prior to administration of the therapeutically effective amount of trifluridine.

In some aspects, after the therapeutically effective amount of trifluridine is administered for at least 28 days, the subject's total bilirubin level does not rise more than about 20% above the subject's total bilirubin level prior to administration of the therapeutically effective amount of trifluridine.

In some aspects, after the therapeutically effective amount of trifluridine is administered for at least 7 days, the subject's total bilirubin level does not rise more than about 1 mg/dL above the subject's total bilirubin level prior to administration of the therapeutically effective amount of trifluridine.

In some aspects, after the therapeutically effective amount of trifluridine is administered for at least 14 days, the subject's total bilirubin level does not rise more than about 1 mg/dL above the subject's total bilirubin level prior to administration of the therapeutically effective amount of trifluridine.

In some aspects, after the therapeutically effective amount of trifluridine is administered for at least 28 days, the subject's total bilirubin level does not rise more than about 1 mg/dL above the subject's total bilirubin level prior to administration of the therapeutically effective amount of trifluridine.

In some aspects, the method of treating liver cancer does not comprise administering radiation therapy.

In some aspects, the subject has a plasma concentration of trifluridine of less than about 0.2 μM after the therapeutically effective amount of trifluridine is administered for at least 7 days.

In some aspects, the subject has a plasma concentration of trifluridine of about 0.07 μM or less after the therapeutically effective amount of trifluridine is administered for at least 7 days.

In some aspects, the subject has a plasma concentration of trifluridine of less than about 0.2 μM after the therapeutically effective amount of trifluridine is administered for at least 14 days.

In some aspects, the subject has a plasma concentration of trifluridine of about 0.07 μM or less after the therapeutically effective amount of trifluridine is administered for at least 14 days.

In some aspects, the subject has a plasma concentration of trifluridine of less than about 0.2 μM after the therapeutically effective amount of trifluridine is administered for at least 28 days.

In some aspects, the subject has a plasma concentration of trifluridine of about 0.07 μM or less after the therapeutically effective amount of trifluridine is administered for at least 28 days.

In some aspects, the method does not comprise filtering the blood of the liver.

In some aspects, the method can further comprise administering to the subject an effective amount of dexamethasone. In some aspects, the effective amount of dexamethasone is an amount effective to function as an anti-inflammatory agent.

In some aspects, the method can further comprise administering to the subject an effective amount of heparin. In some aspects, the effective amount of heparin is an amount effective to prevent clotting of the device, artery, and/or vein.

In some aspects, the method of treating liver cancer can further include administering an effective amount of an additional therapeutic agent. In some aspects, the additional therapeutic agent is a chemotherapeutic agent.

In some aspects, the additional chemotherapeutic agent is floxuridine.

In some aspects, the additional chemotherapeutic agent is fluorouracil.

In some aspects, the method can further comprise administering to the subject an effective amount of a thymidine phosphorylase (TP) inhibitor.

In some aspects, the thymidine phosphorylase (TP) inhibitor is tipiracil.

In some aspects, the method of treating liver cancer does not comprise administering a second agent. In some aspects, the method of treating liver cancer does not comprise administering a chemotherapeutic agent. In some aspects, the method of treating liver cancer does not comprise administering a thymidine phosophorylase (TP) inhibitor. In some aspects, the method of treating liver cancer does not comprise administering the thymidine phosphorylase (TP) inhibitor tipiracil.

The present disclosure further relates to pharmaceutical formulations that contain trifluridine (FTD).

In some aspects, the formulation, e.g., a liquid pharmaceutical formulation, has improved trifluridine solubility and trifluridine stability in an aqueous solution at body temperature. In some of these aspects, the improved formulation increases the daily dose of trifluridine that can be given to a subject in need thereof to increase potential for efficacy.

In some aspects, the formulation comprises about 50 mg/ml to about 150 mg/ml of trifluridine, about 5 mM to about 250 mM of buffer, and a solvent system comprising water and a co-solvent, wherein the formulation has a pH of about 1 to 8.

In some aspects, the formulation comprises about 50 mg/ml to about 100 mg/ml of trifluridine.

In some aspects, the formulation comprises about 50 mg/ml of trifluridine.

In some aspects, the formulation comprises about 70 mg/ml of trifluridine.

In some aspects, the formulation comprises about 100 mg/ml of trifluridine.

In some aspects, the formulation comprises about 65 mg/ml to about 75 mg/ml of trifluridine.

In some aspects, the buffer is selected from the group consisting of a phosphate buffer, borate buffer, acetate buffer, carbonate buffer, maleate buffer, gluconate buffer, tartrate buffer, citrate buffer, or combinations thereof.

In some aspects, the buffer comprises an acetate buffer.

In some aspects, the buffer comprises a citrate buffer.

In some aspects, the formulation comprises about 25 mM to about 100 mM of buffer.

In some aspects, the formulation comprises about 25 mM of buffer.

In some aspects, the formulation comprises about 50 mM of buffer.

In some aspects, the formulation comprises about 75 mM of buffer.

In some aspects, the formulation comprises about 100 mM of buffer.

In some aspects, the co-solvent is selected from the group consisting of ethanol, propylene glycol, dimethyl sulfoxide, dimethylacetamide, and N-methyl-2-pyrrolidone, or combinations thereof.

In some aspects, the solvent system comprises two or more co-solvents.

In some aspects, the co-solvent is ethanol.

In some aspects, the co-solvent is dimethyl sulfoxide.

In some aspects, the co-solvent is dimethylacetamide.

In some aspects, the co-solvent is N-methyl-2-pyrrolidone.

In some aspects, the solvent system comprises about 1% to about 50% v/v of co-solvent.

In some aspects, the solvent system comprises about 10% to about 30% v/v of co-solvent.

In some aspects, the solvent system comprises about 10% to about 20% v/v of co-solvent.

In some aspects, the pH is about 2 to about 5.

In some aspects, the pH is about 3 to about 3.2.

In some aspects, the pH is about 3.

In some aspects, the formulation does not comprise an anticoagulant.

In some aspects, the formulation does not comprise heparin.

In some aspects, the formulation does not comprise fondaparinux.

In some aspects, the formulation comprises an anticoagulant

In some aspects, the formulation comprises heparin.

In some aspects, the formulation comprises fondaparinux.

In some aspects, the formulation comprises about 50 mg/ml to about 100 mg/ml of trifluridine, about 25 mM to about 100 mM of citrate buffer, a solvent system comprising water and about 10% to about 30% v/v dimethylacetamide, wherein the formulation has a pH of about 2 to 5.

In a particular aspect, the formulation comprises about 70 mg/ml of trifluridine, about 75 mM citrate buffer, a solvent system comprising water and about 14% v/v dimethylacetamide, wherein the formulation has a pH of about 3.

The present disclosure also describes a method of treating liver cancer in a subject in need thereof by administering to the subject a therapeutically effective amount of the formulation disclosed herein.

In some aspects, the method can comprise locally administering a therapeutically effective amount of the formulation to the subject.

In some aspects, the method can comprise administering a therapeutically effective amount of the formulation to the subject by intravenous or intra-arterial delivery.

In some aspects, the method can comprise administering to the subject a therapeutically effective amount of the formulation by hepatic arterial infusion.

In some aspects, the therapeutically effective amount of the formulation is administered via a pump.

In some aspects, the therapeutically effective amount of the formulation comprises about 0.01 mg/kg/day to about 5 mg/kg/day of trifluridine.

In some aspects, the therapeutically effective amount of the formulation comprises about 0.1 mg/kg/day to about 2 mg/kg/day of trifluridine.

In some aspects, the therapeutically effective amount of the formulation comprises about 0.5 mg/kg/day to about 1.5 mg/kg/day of trifluridine.

### BRIEF DESCRIPTION OF THE FIGURES

FIG. 1 is a graph showing the efficacy of trifluridine in vitro via a cell cytotoxicity assay using five human cell lines: 1) DLD-1 (colorectal), 2) HCT-116 (colorectal), 3) WiDr (colorectal), 4) Li-7 (hepatocellular carcinoma), and 5) KKU-055 (cholangiocarcinoma). The IC₅₀ of trifluridine was measured on days 1, 3, 7, and 14.

FIGS. 2A-2D are graphs showing liver function in rats when dosed with trifluridine at 0.5 mg/day/kg, 1.5 mg/kg/day, and 4.5 mg/kg/day over 28 days. FIG. 2A shows alanine aminotransferase levels (U/L) over 28 days. FIG. 2B shows aspartate aminotransferase levels (U/L) over 28 days. FIG. 2C shows alkaline phosphatase levels (U/L) over 28 days. FIG. 2D shows bilirubin levels (mg/dL) over 28 days.

FIGS. 3A-3D are graphs showing liver function in rats when dosed with trifluridine at 5 mg/day/kg and 15 mg/kg/day over 28 days. FIG. 3A shows alanine aminotransferase levels (U/L) over 28 days. FIG. 3B shows aspartate aminotransferase levels (U/L) over 28 days. FIG. 3C shows alkaline phosphatase levels (U/L) over 28 days. FIG. 3D shows bilirubin levels (mg/dL) over 28 days.

FIG. 4 is a graph showing the effect of a co-solvent, in particular, dimethylacetamide (DMA), on trifluridine daily dose (mg/day) that can be provided, in formulations with and without heparin.

### DETAILED DESCRIPTION

The present disclosure is directed to methods of treatment of liver cancer by administration of a therapeutically effective amount of trifluridine (FTD) by local administration. The present disclosure is also directed to methods of treatment of liver cancer by administration of a therapeutically effective amount of trifluridine (FTD) by hepatic arterial infusion (HAI).

### Definitions

The singular forms “a,” “an,” and “the” include plural referents unless the context clearly dictates otherwise.

As used herein, the term “or” is a logical disjunction (i.e., and/or) and does not indicate an exclusive disjunction unless expressly indicated as such with the terms “either,” “unless,” “alternatively,” and words of similar effect.

As used herein, the term “about” refers to ±5% of the noted value, unless otherwise specified, and unless the upper bound of the range would exceed 100% of the composition, in which case the upper limit of the range is limited to 99.9%. Thus, and by way of example only, a composition including about 10 weight percent of a given ingredient could have from 9.5 to 10.5 weight percent of the compound. Similarly, a composition including about 95 weight percent of a given ingredient could have from 90.25 to 99.9 weight percent of the ingredient in the composition.

The terms “treat,” “treating,” and “treatment,” as used herein, refer to any type of intervention or process performed on, or administering an active agent to, a subject with the objective of reversing, alleviating, ameliorating, inhibiting, or slowing down the progression, development, severity or recurrence of a symptom, complication, condition or biochemical indicia associated with a disease or enhancing overall survival.

The term “therapeutically effective amount” refers to an amount of a compound that is effective in reducing, eliminating, treating or controlling the herein-described diseases and conditions or the symptoms of the herein-described diseases and conditions.

As used herein, the term “intra-arterial administration” refers to the injection or infusion of liquid substances directly into an artery. This term includes delivery via any type of intra-arterial access device.

As used herein, “U/L” refers to units per liter and “IU/L” refers to international units per liter.

### Methods of Treatment

The disclosure provides a method of treating liver cancer in a subject in need thereof comprising administering to the subject a therapeutically effective amount of trifluridine by local administration. Non-limiting examples of local administration include, but are not limited to, injection into a tumor mass (intratumoral), subcutaneous injection, intramuscular injection, and the like.

In some aspects, the subject has microscopic disease or is more prone to liver metastases such that the administration of a therapeutically effective amount of trifluridine treats microscopic disease (e.g., eliminates microscopic disease) and/or inhibits or slows down the progression from microscopic to macroscopic disease. In some aspects, the subject to be treated is a subject with macroscopic disease.

In some aspects, the subject to be treated is a human. In some aspects, the subject to be treated is a human adult. In some aspects, the subject to be treated is a human child.

In some aspects, the disclosure provides a method of treating liver cancer in a subject in need thereof comprising administering to the subject a therapeutically effective amount of trifluridine by intravenous (IV) or intra-arterial delivery. In some aspects, the therapeutically effective amount of trifluridine is administered by a bolus injection. In some aspects, the therapeutically effective amount of trifluridine is locally administered by a bolus injection. In some aspects, the therapeutically effective amount of trifluridine is administered by a bolus injection to the hepatic artery.

In some aspects, the therapeutically effective amount of trifluridine is locally administered via a bolus injection at a total daily dose from about 0.1 mg to about 10 g. In some aspects, the therapeutically effective amount of trifluridine is locally administered via a bolus injection at a total daily dose from about 0.1 mg to about 5 g, about 0.1 mg to about 4.5 g, about 0.1 mg to about 4 g, about 0.1 mg to about 3.5 g, about 0.1 mg to about 3 g, about 0.1 mg to about 2.5 g, about 0.1 mg to about 2 g, about 0.1 mg to about 1.5 g, about 0.1 mg to about 1 g, about 0.1 mg to about 500 mg, about 0.1 mg to about 250 mg, about 0.1 mg to about 200 mg, about 0.1 mg to about 150 mg, about 0.1 mg to about 100 mg, about 0.1 mg to about 90 mg, about 0.1 mg to about 80 mg, about 0.1 mg to about 70 mg, about 0.1 mg to about 60 mg, about 0.1 mg to about 50 mg, about 0.1 mg to about 40 mg, about 0.1 mg to about 30 mg, about 0.1 mg to about 20 mg, or about 0.1 mg to about 10 mg.

In some aspects, the therapeutically effective amount of trifluridine is locally administered via a bolus injection at a total daily dose from 0.1 mg to 10 g. In some aspects, the therapeutically effective amount of trifluridine is locally administered via a bolus injection at a total daily dose from 0.1 mg to 5 g, 0.1 mg to 4.5 g, 0.1 mg to 4 g, 0.1 mg to 3.5 g, 0.1 mg to 3 g, 0.1 mg to 2.5 g, 0.1 mg to 2 g, 0.1 mg to 1.5 g, 0.1 mg to 1 g, 0.1 mg to 500 mg, 0.1 mg to 250 mg, 0.1 mg to 200 mg, 0.1 mg to 150 mg, 0.1 mg to 100 mg, 0.1 mg to 90 mg, 0.1 mg to 80 mg, 0.1 mg to 70 mg, 0.1 mg to 60 mg, 0.1 mg to 50 mg, 0.1 mg to 40 mg, 0.1 mg to 30 mg, 0.1 mg to 20 mg, or 0.1 mg to 10 mg.

In some aspects, the therapeutically effective amount of trifluridine is locally administered via a bolus injection for about 1 second to about 1 hour. In some aspects, the therapeutically effective amount of trifluridine is locally administered via a bolus injection for about 1 second to about 45 minutes, about 1 second to about 40 minutes, about 1 second to about 35 minutes, about 1 second to about 30 minutes, about 1 second to about 25 minutes, about 1 second to about 20 minutes, about 1 second to about 15 minutes, about 1 second to about 10 minutes, about 1 second to about 5 minutes, about 1 second to about 1 minute, about 1 second to about 45 seconds, about 1 second to about 30 seconds, about 1 second to about 20 seconds, or about 1 second to about 10 seconds. In some aspects, the therapeutically effective amount of trifluridine is locally administered via a bolus injection for about 1 minute to about 60 minutes, about 1 minute to about 45 minutes, about 1 minute to about 40 minutes, about 1 minute to about 35 minutes, about 1 minute to about 30 minutes, about 1 minute to about 25 minutes, about 1 minute to about 20 minutes, about 1 minute to about 15 minutes, about 1 minute to about 10 minutes, or about 1 minute to about 5 minutes. In some aspects, the therapeutically effective amount of trifluridine is locally administered via a bolus injection for about 10 minutes to about 60 minutes, about 10 minutes to about 45 minutes, for about 10 minutes to about 40 minutes, about 10 minutes to about 35 minutes, about 10 minutes to about 30 minutes, about 10 minutes to about 25 minutes, or about 10 minutes to about 20 minutes.

In some aspects, the therapeutically effective amount of trifluridine is locally administered via a bolus injection for 1 second to 1 hour. In some aspects, the therapeutically effective amount of trifluridine is locally administered via a bolus injection for 1 second to 45 minutes, 1 second to 40 minutes, 1 second to 35 minutes, 1 second to 30 minutes, 1 second to 25 minutes, 1 second to 20 minutes, 1 second to 15 minutes, 1 second to 10 minutes, 1 second to 5 minutes, 1 second to 1 minute, 1 second to 45 seconds, 1 second to 30 seconds, 1 second to 20 seconds, or 1 second to 10 seconds. In some aspects, the therapeutically effective amount of trifluridine is locally administered via a bolus injection for 1 minute to 60 minutes, 1 minute to 45 minutes, about 1 minute to 40 minutes, 1 minute to 35 minutes, 1 minute to 30 minutes, 1 minute to 25 minutes, 1 minute to 20 minutes, 1 minute to 15 minutes, 1 minute to 10 minutes, or 1 minute to 5 minutes. In some aspects, the therapeutically effective amount of trifluridine is locally administered via a bolus injection for 10 minutes to 60 minutes, 10 minutes to 45 minutes, for 10 minutes to 40 minutes, 10 minutes to 35 minutes, 10 minutes to 30 minutes, 10 minutes to 25 minutes, or 10 minutes to 20 minutes.

In some aspects, the therapeutically effective amount of trifluridine is administered via a bolus injection to the hepatic artery at a total daily dose from about 0.1 mg to about 10 g. In some aspects, the therapeutically effective amount of trifluridine is administered via a bolus injection to the hepatic artery at a total daily dose from about 0.1 mg to about 5 g, about 0.1 mg to about 4.5 g, about 0.1 mg to about 4 g, about 0.1 mg to about 3.5 g, about 0.1 mg to about 3 g, about 0.1 mg to about 2.5 g, about 0.1 mg to about 2 g, about 0.1 mg to about 1.5 g, about 0.1 mg to about 1 g, about 0.1 mg to about 500 mg, about 0.1 mg to about 250 mg, about 0.1 mg to about 200 mg, about 0.1 mg to about 150 mg, about 0.1 mg to about 100 mg, about 0.1 mg to about 90 mg, about 0.1 mg to about 80 mg, about 0.1 mg to about 70 mg, about 0.1 mg to about 60 mg, about 0.1 mg to about 50 mg, about 0.1 mg to about 40 mg, about 0.1 mg to about 30 mg, about 0.1 mg to about 20 mg, or about 0.1 mg to about 10 mg.

In some aspects, the therapeutically effective amount of trifluridine is administered via a bolus injection to the hepatic artery at a total daily dose from 0.1 mg to 10 g. In some aspects, the therapeutically effective amount of trifluridine is administered via a bolus injection to the hepatic artery at a total daily dose from 0.1 mg to 5 g, 0.1 mg to 4.5 g, 0.1 mg to 4 g, 0.1 mg to 3.5 g, 0.1 mg to 3 g, 0.1 mg to 2.5 g, 0.1 mg to 2 g, 0.1 mg to 1.5 g, 0.1 mg to 1 g, 0.1 mg to 500 mg, 0.1 mg to 250 mg, 0.1 mg to 200 mg, 0.1 mg to 150 mg, 0.1 mg to 100 mg, 0.1 mg to 90 mg, 0.1 mg to 80 mg, 0.1 mg to 70 mg, 0.1 mg to 60 mg, 0.1 mg to 50 mg, 0.1 mg to 40 mg, 0.1 mg to 30 mg, 0.1 mg to 20 mg, or 0.1 mg to 10 mg.

In some aspects, the therapeutically effective amount of trifluridine is administered via a bolus injection to the hepatic artery for about 1 second to about 1 hour. In some aspects, the therapeutically effective amount of trifluridine is administered via a bolus injection to the hepatic artery for about 1 second to about 45 minutes, about 1 second to about 40 minutes, about 1 second to about 35 minutes, about 1 second to about 30 minutes, about 1 second to about 25 minutes, about 1 second to about 20 minutes, about 1 second to about 15 minutes, about 1 second to about 10 minutes, about 1 second to about 5 minutes, about 1 second to about 1 minute, about 1 second to about 45 seconds, about 1 second to about 30 seconds, about 1 second to about 20 seconds, or about 1 second to about 10 seconds. In some aspects, the therapeutically effective amount of trifluridine is administered via a bolus injection to the hepatic artery for about 1 minute to about 60 minutes, about 1 minute to about 45 minutes, about 1 minute to about 40 minutes, about 1 minute to about 35 minutes, about 1 minute to about 30 minutes, about 1 minute to about 25 minutes, about 1 minute to about 20 minutes, about 1 minute to about 15 minutes, about 1 minute to about 10 minutes, or about 1 minute to about 5 minutes. In some aspects, the therapeutically effective amount of trifluridine is administered via a bolus injection to the hepatic artery for about 10 minutes to about 60 minutes, about 10 minutes to about 45 minutes, for about 10 minutes to about 40 minutes, about 10 minutes to about 35 minutes, about 10 minutes to about 30 minutes, about 10 minutes to about 25 minutes, or about 10 minutes to about 20 minutes.

In some aspects, the therapeutically effective amount of trifluridine is administered via a bolus injection to the hepatic artery for 1 second to 1 hour. In some aspects, the therapeutically effective amount of trifluridine is administered via a bolus injection to the hepatic artery for 1 second to 45 minutes, 1 second to 40 minutes, 1 second to 35 minutes, 1 second to 30 minutes, 1 second to 25 minutes, 1 second to 20 minutes, 1 second to 15 minutes, 1 second to 10 minutes, 1 second to 5 minutes, 1 second to 1 minute, 1 second to 45 seconds, 1 second to 30 seconds, 1 second to 20 seconds, or 1 second to 10 seconds. In some aspects, the therapeutically effective amount of trifluridine is administered via a bolus injection to the hepatic artery for 1 minute to 60 minutes, 1 minute to 45 minutes, about 1 minute to 40 minutes, 1 minute to 35 minutes, 1 minute to 30 minutes, 1 minute to 25 minutes, 1 minute to 20 minutes, 1 minute to 15 minutes, 1 minute to 10 minutes, or 1 minute to 5 minutes. In some aspects, the therapeutically effective amount of trifluridine is administered via a bolus injection to the hepatic artery for 10 minutes to 60 minutes, 10 minutes to 45 minutes, for 10 minutes to 40 minutes, 10 minutes to 35 minutes, 10 minutes to 30 minutes, 10 minutes to 25 minutes, or 10 minutes to 20 minutes.

In some aspects, the therapeutically effective amount of trifluridine is administered by intravenous infusion. In some aspects, the therapeutically effective amount of trifluridine is administered by intravenous infusion via a pump. Intravenous infusion pumps known by and available to one of ordinary skill in the art is used to administer trifluridine. Examples of intravenous infusion pumps may include, but are not limited to, Baxter Sigma Spectrum Infusion Pump, BD CareFusion Alaris Medley 8015 PC Infusion Pump, Sigma Spectrum 6.05.14 Wireless B/G, Hospira/Abbott Plum A+13.41 Mednet, and ICU Medical Plum 360 Infusion Pump.

Infusion pumps known by and available to one of ordinary skill in the art is used to administer trifluridine, including, but not limited to, implantable pumps, external pumps, continuous flow pumps, and programmable pumps.

In some aspects, the therapeutically effective amount of trifluridine is administered by intra-arterial injection. In some aspects, the therapeutically effective amount of trifluridine is administered by intra-arterial infusion. In some aspects, the therapeutically effective amount of trifluridine is administered by intra-arterial infusion via a pump. Intra-arterial infusion pumps known by and available to one of ordinary skill in the art is used to administer trifluridine, including implantable pumps, external pumps, continuous flow pumps, and programmable pumps.

In some aspects, the therapeutically effective amount of trifluridine is administered by hepatic arterial delivery. In some aspects, the therapeutically effective amount of trifluridine is administered by hepatic arterial injection. In some aspects, the therapeutically effective amount of trifluridine is administered by hepatic arterial infusion. In some aspects, the therapeutically effective amount of trifluridine is administered by hepatic arterial infusion via a pump. Hepatic artery infusion pumps known by and available to one of ordinary skill in the art is used to administer trifluridine, including, but not limited to, implantable pumps, external pumps, continuous flow pumps, and programmable pumps. Examples of hepatic artery infusion pumps may include, but are not limited to, Intera 3000 Hepatic Artery Infusion (HAI) Pump.

In some aspects, the therapeutically effective amount of trifluridine is administered through the hepatic portal vein. In some aspects, the therapeutically effective amount of trifluridine is administered by hepatic portal vein injection. In some aspects, the therapeutically effective amount of trifluridine is administered by hepatic portal vein infusion. In some aspects, the therapeutically effective amount of trifluridine is administered by hepatic portal vein infusion via a pump. Infusion pumps known by and available to one of ordinary skill in the art is used to administer trifluridine, including, but not limited to, implantable pumps, external pumps, continuous flow pumps, and programmable pumps.

In some aspects, the therapeutically effective amount of trifluridine is continuously administered for an indefinite period of time. In some aspects, the therapeutically effective amount of trifluridine is continuously administered by intravenous or intra-arterial delivery. In some aspects, the therapeutically effective amount of trifluridine is continuously administered by hepatic portal vein infusion. In some aspects, the therapeutically effective amount of trifluridine is continuously administered by hepatic arterial infusion. In some aspects, the therapeutically effective amount of trifluridine is continuously administered for about 3 years or more. In some aspects, the therapeutically effective amount of trifluridine is continuously administered for about 2 years or more. In some aspects, the therapeutically effective amount of trifluridine is continuously administered for about a year or more. In some aspects, the therapeutically effective amount of trifluridine is continuously administered for about 9 months or more. In some aspects, the therapeutically effective amount of trifluridine is continuously administered for about 6 months or more. In some aspects, the therapeutically effective amount of trifluridine is continuously administered for about 3 months or more. In some aspects, the therapeutically effective amount of trifluridine is continuously administered for about 2 months or more. In some aspects, the therapeutically effective amount of trifluridine is continuously administered for about 1 month or more. In some aspects, the therapeutically effective amount of trifluridine is continuously administered until the cancer is treated or the desired therapeutic effect is achieved. In some aspects, the therapeutically effective amount of trifluridine is continuously administered up to the lifetime of the subject.

In some aspects, the therapeutically effective amount of trifluridine is continuously administered for at least 3 years or more. In some aspects, the therapeutically effective amount of trifluridine is continuously administered for at least 2 years or more. In some aspects, the therapeutically effective amount of trifluridine is continuously administered for at least a year or more. In some aspects, the therapeutically effective amount of trifluridine is continuously administered for at least 9 months or more. In some aspects, the therapeutically effective amount of trifluridine is continuously administered for at least 6 months or more. In some aspects, the therapeutically effective amount of trifluridine is continuously administered for at least 3 months or more. In some aspects, the therapeutically effective amount of trifluridine is continuously administered for at least 2 months or more. In some aspects, the therapeutically effective amount of trifluridine is continuously administered for at least 1 month or more. In some aspects, the therapeutically effective amount of trifluridine is continuously administered until the cancer is treated or the desired therapeutic effect is achieved. In some aspects, the therapeutically effective amount of trifluridine is continuously administered up to the lifetime of the subject.

In some aspects, the therapeutically effective amount of trifluridine is continuously administered for 3 years or more. In some aspects, the therapeutically effective amount of trifluridine is continuously administered for 2 years or more. In some aspects, the therapeutically effective amount of trifluridine is continuously administered for at least a year or more. In some aspects, the therapeutically effective amount of trifluridine is continuously administered for 9 months or more. In some aspects, the therapeutically effective amount of trifluridine is continuously administered for 6 months or more. In some aspects, the therapeutically effective amount of trifluridine is continuously administered for 3 months or more. In some aspects, the therapeutically effective amount of trifluridine is continuously administered for 2 months or more. In some aspects, the therapeutically effective amount of trifluridine is continuously administered for 1 month or more.

In some aspects, the therapeutically effective amount of trifluridine is continuously administered for 1 week. In some aspects, the therapeutically effective amount of trifluridine is continuously administered for 2 weeks. In some aspects, the therapeutically effective amount of trifluridine is continuously administered for 3 weeks. In some aspects, the therapeutically effective amount of trifluridine is continuously administered for 4 weeks. In some aspects, the therapeutically effective amount of trifluridine is continuously administered for 1 month. In some aspects, the therapeutically effective amount of trifluridine is continuously administered for 5 weeks. In some aspects, the therapeutically effective amount of trifluridine is continuously administered for 6 weeks. In some aspects, the therapeutically effective amount of trifluridine is continuously administered for 7 weeks. In some aspects, the therapeutically effective amount of trifluridine is continuously administered for 8 weeks. In some aspects, the therapeutically effective amount of trifluridine is continuously administered for 2 months. In some aspects, the therapeutically effective amount of trifluridine is continuously administered for 3 months. In some aspects, the therapeutically effective amount of trifluridine is continuously administered for 6 months. In some aspects, the therapeutically effective amount of trifluridine is continuously administered for 9 months. In some aspects, the therapeutically effective amount of trifluridine is continuously administered for 1 year. In some aspects, the therapeutically effective amount of trifluridine is continuously administered for 2 years.

In some aspects, the therapeutically effective amount of trifluridine is continuously administered for about 1 month to about 3 years. In some aspects, the therapeutically effective amount of trifluridine is continuously administered for about 1 month to about 2 years. In some aspects, the therapeutically effective amount of trifluridine is continuously administered for about 1 month to about 1 year. In some aspects, the therapeutically effective amount of trifluridine is continuously administered for about 1 month to about 9 months. In some aspects, the therapeutically effective amount of trifluridine is continuously administered for about 1 month to about 6 months. In some aspects, the therapeutically effective amount of trifluridine is continuously administered for about 1 month to about 3 months. In some aspects, the therapeutically effective amount of trifluridine is continuously administered for about 1 month to about 2 months.

In some aspects, the therapeutically effective amount of trifluridine is continuously administered for 1 month to 3 years. In some aspects, the therapeutically effective amount of trifluridine is continuously administered for 1 month to 2 years. In some aspects, the therapeutically effective amount of trifluridine is continuously administered for 1 month to 1 year. In some aspects, the therapeutically effective amount of trifluridine is continuously administered for 1 month to 9 months. In some aspects, the therapeutically effective amount of trifluridine is continuously administered for 1 month to 6 months. In some aspects, the therapeutically effective amount of trifluridine is continuously administered for 1 month to 3 months. In some aspects, the therapeutically effective amount of trifluridine is continuously administered for 1 month to 2 months.

In some aspects, the therapeutically effective amount of trifluridine is continuously administered for about 30 minutes. In some aspects, the therapeutically effective amount of trifluridine is continuously administered for about 1 hour, about 2 hours, about 3 hours, about 4 hours, about 8 hours, about 12 hours, about 24 hours, about 48 hours, about 72 hours, about 7 days, about 14 days, about 28 days, about a week, about 2 weeks, about 3 months, about a month.

In some aspects, the therapeutically effective amount of trifluridine is continuously administered for at least 30 minutes. In some aspects, the therapeutically effective amount of trifluridine is continuously administered for at least 1 hour, at least 2 hours, at least 3 hours, at least 4 hours, at least 8 hours, at least 12 hours, at least 24 hours, at least 48 hours, at least 72 hours, at least 7 days, at least 14 days, at least 28 days, at least a week, at least 2 weeks, at least 3 months, at least a month.

In some aspects, the therapeutically effective amount of trifluridine is continuously administered for about 30 minutes to about 3 months. In some aspects, the therapeutically effective amount of trifluridine is continuously administered for about 30 minutes to about 2 months. In some aspects, the therapeutically effective amount of trifluridine is continuously administered for about 30 minutes to about 1 month.

In some aspects, the therapeutically effective amount of trifluridine is continuously administered for about 7 days, about 14 days, or about 28 days. In some aspects, the therapeutically effective amount of trifluridine is continuously administered by intravenous or intra-arterial infusion until the cancer is treated or the desired therapeutic effect is achieved. In some aspects, the therapeutically effective amount of trifluridine is continuously administered by intravenous or intra-arterial infusion up to the lifetime of the subject.

In some aspects, the therapeutically effective amount of trifluridine is continuously administered for at least 7 days, at least 14 days, or at least 28 days. In some aspects, the therapeutically effective amount of trifluridine is continuously administered by intravenous or intra-arterial infusion until the cancer is treated or the desired therapeutic effect is achieved. In some aspects, the therapeutically effective amount of trifluridine is continuously administered by intravenous or intra-arterial infusion up to the lifetime of the subject.

In some aspects, the therapeutically effective amount of trifluridine is continuously administered by the infusion techniques described herein via a pump. In some aspects, the pump is internal. In some aspects, the internal pump is refilled about every 2 to 4 weeks to maintain continuous administration. In some aspects, the internal pump is refilled about every 2 to 3 weeks. In some aspects, the internal pump is refilled every week. In some aspects, the internal pump is refilled about every 2 weeks. In some aspects, the internal pump is refilled about every 3 weeks. In some aspects, the internal pump is refilled about every 4 weeks.

In some aspects, the therapeutically effective amount of trifluridine is continuously administered by the infusion techniques described herein via a pump. In some aspects, the pump is external. In some aspects, the external pump is refilled about every 2 to 4 weeks to maintain continuous administration. In some aspects, the external pump is refilled about every 2 to 3 weeks. In some aspects, the external pump is refilled every week. In some aspects, the external pump is refilled about every 2 weeks. In some aspects, the external pump is refilled about every 3 weeks. In some aspects, the external pump is refilled about every 4 weeks.

In some aspects, the therapeutically effective amount of trifluridine is administered via an internal pump implanted into the subject. In some aspects, the subject is a human. In some aspects, the therapeutically effective amount of trifluridine is administered via an internal pump in an amount of about 0.01 mg/kg/day to about 10 mg/kg/day. In some aspects, the therapeutically effective amount of trifluridine is administered via an internal pump in an amount of about 0.01 mg/kg/day to about 9 mg/kg/day, about 0.01 mg/kg/day to about 8 mg/kg/day, about 0.01 mg/kg/day to about 7 mg/kg/day, about 0.01 mg/kg/day to about 6 mg/kg/day, from about 0.01 mg/kg/day to about 5 mg/kg/day, about 0.01 mg/kg/day to about 4 mg/kg/day, about 0.01 mg/kg/day to about 3 mg/kg/day, about 0.01 mg/kg/day to about 2 mg/kg/day, about 0.01 mg/kg/day to about 1 mg/kg/day, about 0.01 mg/kg/day to about 0.9 mg/kg/day, about 0.01 mg/kg/day to about 0.8 mg/kg/day, about 0.01 mg/kg/day to about 0.7 mg/kg/day, about 0.01 mg/kg/day to about 0.6 mg/kg/day, about 0.01 mg/kg/day to about 0.5 mg/kg/day, about 0.01 mg/kg/day to about 0.4 mg/kg/day, about 0.01 mg/kg/day to about 0.3 mg/kg/day, about 0.01 mg/kg/day to about 0.2 mg/kg/day, or about 0.01 mg/kg/day to about 0.1 mg/kg/day.

In some aspects, the therapeutically effective amount of trifluridine is administered via an internal pump in an amount of about 0.1 mg/kg/day to about 10 mg/kg/day. In some aspects, the therapeutically effective amount of trifluridine is administered via an internal pump in an amount of about 0.1 mg/kg/day to about 9 mg/kg/day, about 0.1 mg/kg/day to about 8 mg/kg/day, about 0.1 mg/kg/day to about 7 mg/kg/day, about 0.1 mg/kg/day to about 6 mg/kg/day, 0.1 mg/kg/day to about 5 mg/kg/day, about 0.1 mg/kg/day to about 4 mg/kg/day, about 0.1 mg/kg/day to about 3 mg/kg/day, about 0.1 mg/kg/day to about 2 mg/kg/day, about 0.1 mg/kg/day to about 1 mg/kg/day, about 0.1 mg/kg/day to about 0.9 mg/kg/day, about 0.1 mg/kg/day to about 0.8 mg/kg/day, about 0.1 mg/kg/day to about 0.7 mg/kg/day, about 0.1 mg/kg/day to about 0.6 mg/kg/day, about 0.1 mg/kg/day to about 0.5 mg/kg/day, about 0.1 mg/kg/day to about 0.4 mg/kg/day, about 0.1 mg/kg/day to about 0.3 mg/kg/day, or about 0.1 mg/kg/day to about 0.2 mg/kg/day.

In some aspects, the therapeutically effective amount of trifluridine is administered via an internal pump in an amount of from about 0.7 mg/kg/day to about 10 mg/kg/day. In some aspects, the therapeutically effective amount of trifluridine is administered via an internal pump in an amount of 0.7 mg/kg/day to about 9 mg/kg/day, about 0.7 mg/kg/day to about 8 mg/kg/day, about 0.7 mg/kg/day to about 7 mg/kg/day, about 0.7 mg/kg/day to about 6 mg/kg/day, 0.7 mg/kg/day to about 5 mg/kg/day, about 0.7 mg/kg/day to about 4 mg/kg/day, about 0.7 mg/kg/day to about 3 mg/kg/day, about 0.7 mg/kg/day to about 2 mg/kg/day, about 0.7 mg/kg/day to about 1 mg/kg/day, about 0.7 mg/kg/day to about 0.9 mg/kg/day, or about 0.7 mg/kg/day to about 0.8 mg/kg/day.

In some aspects, the therapeutically effective amount of trifluridine is administered via an internal pump in an amount of from about 1 mg/kg/day to about 10 mg/kg/day. In some aspects, the therapeutically effective amount of trifluridine is administered via an internal pump in an amount of about 1 mg/kg/day to about 9 mg/kg/day, about 1 mg/kg/day to about 8 mg/kg/day, about 1 mg/kg/day to about 7 mg/kg/day, about 1 mg/kg/day to about 6 mg/kg/day, 1 mg/kg/day to about 5 mg/kg/day, about 1 mg/kg/day to about 4 mg/kg/day, about 1 mg/kg/day to about 3 mg/kg/day, about 1 mg/kg/day to about 2 mg/kg/day, or about 1 mg/kg/day to about 1.5 mg/kg/day. In some aspects, the total daily dose is adjusted according to individual patient requirements.

In some aspects, the therapeutically effective amount of trifluridine is administered via an internal pump at a total daily dose from about 0.1 mg to about 250 mg. In some aspects, the therapeutically effective amount of trifluridine is administered via an internal pump at a total daily dose from about 0.1 mg to about 200 mg, about 0.1 mg to about 150 mg, about 0.1 mg to about 100 mg, about 0.1 mg to about 90 mg, about 0.1 mg to about 80 mg, about 0.1 mg to about 70 mg, about 0.1 mg to about 60 mg, about 0.1 mg to about 50 mg, about 0.1 mg to about 40 mg, about 0.1 mg to about 30 mg, about 0.1 mg to about 20 mg, or about 0.1 mg to about 10 mg.

In some aspects, the therapeutically effective amount of trifluridine is administered via an internal pump at a total daily dose from 0.1 mg to 250 mg. In some aspects, the therapeutically effective amount of trifluridine is administered via an internal pump at a total daily dose from 0.1 mg to 200 mg, 0.1 mg to 150 mg, 0.1 mg to 100 mg, 0.1 mg to 90 mg, 0.1 mg to 80 mg, 0.1 mg to 70 mg, 0.1 mg to 60 mg, 0.1 mg to 50 mg, 0.1 mg to 40 mg, 0.1 mg to 30 mg, 0.1 mg to 20 mg, or 0.1 mg to 10 mg.

In some aspects, the therapeutically effective amount of trifluridine is administered via an internal pump at a total daily dose of 250 mg. In some aspects, the therapeutically effective amount of trifluridine is administered via an internal pump at a total daily dose of 200 mg. In some aspects, the therapeutically effective amount of trifluridine is administered via an internal pump at a total daily dose of 100 mg. In some aspects, the therapeutically effective amount of trifluridine is administered via an internal pump at a total daily dose of 90 mg. In some aspects, the therapeutically effective amount of trifluridine is administered via an internal pump at a total daily dose of 80 mg. In some aspects, the therapeutically effective amount of trifluridine is administered via an internal pump at a total daily dose of 70 mg. In some aspects, the therapeutically effective amount of trifluridine is administered via an internal pump at a total daily dose of 60 mg. In some aspects, the therapeutically effective amount of trifluridine is administered via an internal pump at a total daily dose of 50 mg. In some aspects, the therapeutically effective amount of trifluridine is administered via an internal pump at a total daily dose of 40 mg. In some aspects, the therapeutically effective amount of trifluridine is administered via an internal pump at a total daily dose of 30 mg. In some aspects, the therapeutically effective amount of trifluridine is administered via an internal pump at a total daily dose of 20 mg. In some aspects, the therapeutically effective amount of trifluridine is administered via an internal pump at a total daily dose of 10 mg. In some aspects, the therapeutically effective amount of trifluridine is administered via an internal pump at a total daily dose of 1 mg. In some aspects, the therapeutically effective amount of trifluridine is administered via an internal pump at a total daily dose of 0.1 mg.

In some aspects, the therapeutically effective amount of trifluridine is administered via an external pump in an amount of about 0.01 mg/kg/day to about 250 mg/kg/day. In some aspects, the therapeutically effective amount of trifluridine is administered via an external pump in an amount of about 0.01 mg/kg/day to about 200 mg/kg/day, about 0.01 mg/kg/day to about 150 mg/kg/day, about 0.01 mg/kg/day to about 100 mg/kg/day, about 0.01 mg/kg/day to about 50 mg/kg/day, from about 0.01 mg/kg/day to about 25 mg/kg/day, or about 0.01 mg/kg/day to about 10 mg/kg/day.

In some aspects, the therapeutically effective amount of trifluridine is administered via an external pump in an amount of about 0.1 mg/kg/day to about 250 mg/kg/day. In some aspects, the therapeutically effective amount of trifluridine is administered via an external pump in an amount of about 0.1 mg/kg/day to about 200 mg/kg/day, about 0.1 mg/kg/day to about 150 mg/kg/day, about 0.1 mg/kg/day to about 100 mg/kg/day, about 0.1 mg/kg/day to about 50 mg/kg/day, about 0.1 mg/kg/day to about 25 mg/kg/day, or about 0.1 mg/kg/day to about 10 mg/kg/day.

In some aspects, the therapeutically effective amount of trifluridine is administered via an external pump in an amount of about 0.7 mg/kg/day to about 250 mg/kg/day. In some aspects, the therapeutically effective amount of trifluridine is administered via an external pump in an amount of about 0.7 mg/kg/day to about 200 mg/kg/day, about 0.7 mg/kg/day to about 150 mg/kg/day, about 0.7 mg/kg/day to about 100 mg/kg/day mg/kg/day, about 0.7 mg/kg/day to about 50 mg/kg/day, about 0.7 mg/kg/day to about 25 mg/kg/day, or about 0.7 mg/kg/day to about 10 mg/kg/day.

In some aspects, the therapeutically effective amount of trifluridine is administered via an external pump in an amount of about 1 mg/kg/day to about 250 mg/kg/day. In some aspects, the therapeutically effective amount of trifluridine is administered via an external pump in an amount of about 1 mg/kg/day to about 200 mg/kg/day, about 1 mg/kg/day to about 150 mg/kg/day, about 1 mg/kg/day to about 100 mg/kg/day mg/kg/day, about 1 mg/kg/day to about 50 mg/kg/day, about 1 mg/kg/day to about 25 mg/kg/day, or about 1 mg/kg/day to about 10 mg/kg/day.

In some aspects, the therapeutically effective amount of trifluridine is administered via an external pump at a total daily dose from about 0.1 mg to about 20 g. In some aspects, the therapeutically effective amount of trifluridine is administered via an external pump at a total daily dose from about 0.1 mg to about 15 g, about 0.1 mg to about 10 g, about 0.1 mg to about 5 g, about 0.1 mg to about 1 g, about 0.1 mg to about 500 mg, about 0.1 mg to about 250 mg, about 0.1 mg to about 100 mg, about 0.1 mg to about 90 mg, about 0.1 mg to about 80 mg, about 0.1 mg to about 70 mg, about 0.1 mg to about 60 mg, about 0.1 mg to about 50 mg, about 0.1 mg to about 40 mg, about 0.1 mg to about 30 mg, about 0.1 mg to about 20 mg, or about 0.1 mg to about 10 mg.

In some aspects, the therapeutically effective amount of trifluridine is administered via an external pump at a total daily dose from 0.1 mg to 20 g. In some aspects, the therapeutically effective amount of trifluridine is administered via an external pump at a total daily dose of 0.1 mg to 15 g, 0.1 mg to 10 g, 0.1 mg to 5 g, 0.1 mg to 1 g, 0.1 mg to 500 mg, 0.1 mg to 250 mg, 0.1 mg to 100 mg, 0.1 mg to 90 mg, 0.1 mg to 80 mg, 0.1 mg to 70 mg, 0.1 mg to 60 mg, 0.1 mg to 50 mg, 0.1 mg to 40 mg, 0.1 mg to 30 mg, 0.1 mg to 20 mg, or 0.1 mg to 10 mg.

In some aspects, after the therapeutically effective amount of trifluridine is administered for at least 1 day, the subject's alanine aminotransferase level does not rise more than about 20% above the subject's alanine aminotransferase level prior to administration of the therapeutically effective amount of trifluridine. In some aspects, after the therapeutically effective amount of trifluridine is administered for at least 1 day, the subject's alanine aminotransferase level does not rise more than about 15%, more than about 10%, or more than about 5% above the subject's alanine aminotransferase level prior to administration of the therapeutically effective amount of trifluridine.

In some aspects, after the therapeutically effective amount of trifluridine is administered for at least 1 day, the subject's alanine aminotransferase level does not rise more than 20% above the subject's alanine aminotransferase level prior to administration of the therapeutically effective amount of trifluridine. In some aspects, after the therapeutically effective amount of trifluridine is administered for at least 1 day, the subject's alanine aminotransferase level does not rise more than 15%, more than 10%, or more than 5% above the subject's alanine aminotransferase level prior to administration of the therapeutically effective amount of trifluridine.

In some aspects, after the therapeutically effective amount of trifluridine is administered for at least 3 days, the subject's alanine aminotransferase level does not rise more than about 20% above the subject's alanine aminotransferase level prior to administration of the therapeutically effective amount of trifluridine. In some aspects, after the therapeutically effective amount of trifluridine is administered for at least 3 days, the subject's alanine aminotransferase level does not rise more than about 15%, more than about 10%, or more than about 5% above the subject's alanine aminotransferase level prior to administration of the therapeutically effective amount of trifluridine.

In some aspects, after the therapeutically effective amount of trifluridine is administered for at least 3 days, the subject's alanine aminotransferase level does not rise more than 20% above the subject's alanine aminotransferase level prior to administration of the therapeutically effective amount of trifluridine. In some aspects, after the therapeutically effective amount of trifluridine is administered for at least 3 days, the subject's alanine aminotransferase level does not rise more than 15%, more than 10%, or more than 5% above the subject's alanine aminotransferase level prior to administration of the therapeutically effective amount of trifluridine.

In some aspects, after the therapeutically effective amount of trifluridine is administered for at least 7 days, the subject's alanine aminotransferase level does not rise more than about 20% above the subject's alanine aminotransferase level prior to administration of the therapeutically effective amount of trifluridine. In some aspects, after the therapeutically effective amount of trifluridine is administered for at least 7 days, the subject's alanine aminotransferase level does not rise more than about 15%, more than about 10%, or more than about 5% above the subject's alanine aminotransferase level prior to administration of the therapeutically effective amount of trifluridine.

In some aspects, after the therapeutically effective amount of trifluridine is administered for at least 7 days, the subject's alanine aminotransferase level does not rise more than 20% above the subject's alanine aminotransferase level prior to administration of the therapeutically effective amount of trifluridine. In some aspects, after the therapeutically effective amount of trifluridine is administered for at least 7 days, the subject's alanine aminotransferase level does not rise more than 15%, more than 10%, or more than 5% above the subject's alanine aminotransferase level prior to administration of the therapeutically effective amount of trifluridine.

In some aspects, after the therapeutically effective amount of trifluridine is administered for at least 14 days, the subject's alanine aminotransferase level does not rise more than about 20% above the subject's alanine aminotransferase level prior to administration of the therapeutically effective amount of trifluridine. In some aspects, after the therapeutically effective amount of trifluridine is administered for at least 14 days, the subject's alanine aminotransferase level does not rise more than about 15%, more than about 10%, or more than about 5% above the subject's alanine aminotransferase level prior to administration of the therapeutically effective amount of trifluridine.

In some aspects, after the therapeutically effective amount of trifluridine is administered for at least 14 days, the subject's alanine aminotransferase level does not rise more than 20% above the subject's alanine aminotransferase level prior to administration of the therapeutically effective amount of trifluridine. In some aspects, after the therapeutically effective amount of trifluridine is administered for at least 14 days, the subject's alanine aminotransferase level does not rise more than 15%, more than 10%, or more than 5% above the subject's alanine aminotransferase level prior to administration of the therapeutically effective amount of trifluridine.

In some aspects, after the therapeutically effective amount of trifluridine is administered for at least 28 days, the subject's alanine aminotransferase level does not rise more than about 20% above the subject's alanine aminotransferase level prior to administration of the therapeutically effective amount of trifluridine. In some aspects, after the therapeutically effective amount of trifluridine is administered for at least 28 days, the subject's alanine aminotransferase level does not rise more than about 15%, more than about 10%, or more than about 5% above the subject's alanine aminotransferase level prior to administration of the therapeutically effective amount of trifluridine.

In some aspects, after the therapeutically effective amount of trifluridine is administered for at least 28 days, the subject's alanine aminotransferase level does not rise more than 20% above the subject's alanine aminotransferase level prior to administration of the therapeutically effective amount of trifluridine. In some aspects, after the therapeutically effective amount of trifluridine is administered for at least 28 days, the subject's alanine aminotransferase level does not rise more than 15%, more than 10%, or more than 5% above the subject's alanine aminotransferase level prior to administration of the therapeutically effective amount of trifluridine.

In some aspects, after the therapeutically effective amount of trifluridine is administered for at least 2 months, the subject's alanine aminotransferase level does not rise more than about 20% above the subject's alanine aminotransferase level prior to administration of the therapeutically effective amount of trifluridine. In some aspects, after the therapeutically effective amount of trifluridine is administered for at least 2 months, the subject's alanine aminotransferase level does not rise more than about 15%, more than about 10%, or more than about 5% above the subject's alanine aminotransferase level prior to administration of the therapeutically effective amount of trifluridine.

In some aspects, after the therapeutically effective amount of trifluridine is administered for at least 2 months, the subject's alanine aminotransferase level does not rise more than 20% above the subject's alanine aminotransferase level prior to administration of the therapeutically effective amount of trifluridine. In some aspects, after the therapeutically effective amount of trifluridine is administered for at least 2 months, the subject's alanine aminotransferase level does not rise more than 15%, more than 10%, or more than 5% above the subject's alanine aminotransferase level prior to administration of the therapeutically effective amount of trifluridine.

In some aspects, after the therapeutically effective amount of trifluridine is administered for at least 3 months, the subject's alanine aminotransferase level does not rise more than about 20% above the subject's alanine aminotransferase level prior to administration of the therapeutically effective amount of trifluridine. In some aspects, after the therapeutically effective amount of trifluridine is administered for at least 3 months, the subject's alanine aminotransferase level does not rise more than about 15%, more than about 10%, or more than about 5% above the subject's alanine aminotransferase level prior to administration of the therapeutically effective amount of trifluridine.

In some aspects, after the therapeutically effective amount of trifluridine is administered for at least 3 months, the subject's alanine aminotransferase level does not rise more than 20% above the subject's alanine aminotransferase level prior to administration of the therapeutically effective amount of trifluridine. In some aspects, after the therapeutically effective amount of trifluridine is administered for at least 3 months, the subject's alanine aminotransferase level does not rise more than 15%, more than 10%, or more than 5% above the subject's alanine aminotransferase level prior to administration of the therapeutically effective amount of trifluridine.

In some aspects, after the therapeutically effective amount of trifluridine is administered for at least 6 months, the subject's alanine aminotransferase level does not rise more than about 20% above the subject's alanine aminotransferase level prior to administration of the therapeutically effective amount of trifluridine. In some aspects, after the therapeutically effective amount of trifluridine is administered for at least 6 months, the subject's alanine aminotransferase level does not rise more than about 15%, more than about 10%, or more than about 5% above the subject's alanine aminotransferase level prior to administration of the therapeutically effective amount of trifluridine.

In some aspects, after the therapeutically effective amount of trifluridine is administered for at least 6 months, the subject's alanine aminotransferase level does not rise more than 20% above the subject's alanine aminotransferase level prior to administration of the therapeutically effective amount of trifluridine. In some aspects, after the therapeutically effective amount of trifluridine is administered for at least 6 months, the subject's alanine aminotransferase level does not rise more than 15%, more than 10%, or more than 5% above the subject's alanine aminotransferase level prior to administration of the therapeutically effective amount of trifluridine.

In some aspects, after the therapeutically effective amount of trifluridine is administered for at least 1 year, the subject's alanine aminotransferase level does not rise more than about 20% above the subject's alanine aminotransferase level prior to administration of the therapeutically effective amount of trifluridine. In some aspects, after the therapeutically effective amount of trifluridine is administered for at least 1 year, the subject's alanine aminotransferase level does not rise more than about 15%, more than about 10%, or more than about 5% above the subject's alanine aminotransferase level prior to administration of the therapeutically effective amount of trifluridine.

In some aspects, after the therapeutically effective amount of trifluridine is administered for at least 1 year, the subject's alanine aminotransferase level does not rise more than 20% above the subject's alanine aminotransferase level prior to administration of the therapeutically effective amount of trifluridine. In some aspects, after the therapeutically effective amount of trifluridine is administered for at least 1 year, the subject's alanine aminotransferase level does not rise more than 15%, more than 10%, or more than 5% above the subject's alanine aminotransferase level prior to administration of the therapeutically effective amount of trifluridine.

In some aspects, after the therapeutically effective amount of trifluridine is administered for at least 2 years, the subject's alanine aminotransferase level does not rise more than about 20% above the subject's alanine aminotransferase level prior to administration of the therapeutically effective amount of trifluridine. In some aspects, after the therapeutically effective amount of trifluridine is administered for at least 2 years, the subject's alanine aminotransferase level does not rise more than about 15%, more than about 10%, or more than about 5% above the subject's alanine aminotransferase level prior to administration of the therapeutically effective amount of trifluridine.

In some aspects, after the therapeutically effective amount of trifluridine is administered for at least 2 years, the subject's alanine aminotransferase level does not rise more than 20% above the subject's alanine aminotransferase level prior to administration of the therapeutically effective amount of trifluridine. In some aspects, after the therapeutically effective amount of trifluridine is administered for at least 2 years, the subject's alanine aminotransferase level does not rise more than 15%, more than 10%, or more than 5% above the subject's alanine aminotransferase level prior to administration of the therapeutically effective amount of trifluridine.

In some aspects, after the therapeutically effective amount of trifluridine is administered for at least 1 day, the subject's alanine aminotransferase level does not rise more than about 20 U/L above the subject's alanine aminotransferase level prior to administration of the therapeutically effective amount of trifluridine. In some aspects, after the therapeutically effective amount of trifluridine is administered for at least 1 day, the subject's alanine aminotransferase level does not rise more than about 15 U/L, more than about 10 U/L, or more than about 5 U/L above the subject's alanine aminotransferase level prior to administration of the therapeutically effective amount of trifluridine.

In some aspects, after the therapeutically effective amount of trifluridine is administered for at least 1 day, the subject's alanine aminotransferase level does not rise more than 20 U/L above the subject's alanine aminotransferase level prior to administration of the therapeutically effective amount of trifluridine. In some aspects, after the therapeutically effective amount of trifluridine is administered for at least 1 day, the subject's alanine aminotransferase level does not rise more than 15 U/L, more than 10 U/L, or more than 5 U/L above the subject's alanine aminotransferase level prior to administration of the therapeutically effective amount of trifluridine.

In some aspects, after the therapeutically effective amount of trifluridine is administered for at least 3 days, the subject's alanine aminotransferase level does not rise more than about 20 U/L above the subject's alanine aminotransferase level prior to administration of the therapeutically effective amount of trifluridine. In some aspects, after the therapeutically effective amount of trifluridine is administered for at least 3 days, the subject's alanine aminotransferase level does not rise more than about 15 U/L, more than about 10 U/L, or more than about 5 U/L above the subject's alanine aminotransferase level prior to administration of the therapeutically effective amount of trifluridine.

In some aspects, after the therapeutically effective amount of trifluridine is administered for at least 7 days, the subject's alanine aminotransferase level does not rise more than 20 U/L above the subject's alanine aminotransferase level prior to administration of the therapeutically effective amount of trifluridine. In some aspects, after the therapeutically effective amount of trifluridine is administered for at least 7 days, the subject's alanine aminotransferase level does not rise more than 15 U/L, more than 10 U/L, or more than 5 U/L above the subject's alanine aminotransferase level prior to administration of the therapeutically effective amount of trifluridine.

In some aspects, after the therapeutically effective amount of trifluridine is administered for at least 7 days, the subject's alanine aminotransferase level does not rise more than about 20 U/L above the subject's alanine aminotransferase level prior to administration of the therapeutically effective amount of trifluridine. In some aspects, after the therapeutically effective amount of trifluridine is administered for at least 7 days, the subject's alanine aminotransferase level does not rise more than about 15 U/L, more than about 10 U/L, or more than about 5 U/L above the subject's alanine aminotransferase level prior to administration of the therapeutically effective amount of trifluridine.

In some aspects, after the therapeutically effective amount of trifluridine is administered for at least 7 days, the subject's alanine aminotransferase level does not rise more than 20 U/L above the subject's alanine aminotransferase level prior to administration of the therapeutically effective amount of trifluridine. In some aspects, after the therapeutically effective amount of trifluridine is administered for at least 7 days, the subject's alanine aminotransferase level does not rise more than 15 U/L, more than 10 U/L, or more than 5 U/L above the subject's alanine aminotransferase level prior to administration of the therapeutically effective amount of trifluridine.

In some aspects, after the therapeutically effective amount of trifluridine is administered for at least 14 days, the subject's alanine aminotransferase level does not rise more than about 20 U/L above the subject's alanine aminotransferase level prior to administration of the therapeutically effective amount of trifluridine. In some aspects, after the therapeutically effective amount of trifluridine is administered for at least 14 days, the subject's alanine aminotransferase level does not rise more than about 15 U/L, more than about 10 U/L, or more than about 5 U/L above the subject's alanine aminotransferase level prior to administration of the therapeutically effective amount of trifluridine.

In some aspects, after the therapeutically effective amount of trifluridine is administered for at least 14 days, the subject's alanine aminotransferase level does not rise more than 20 U/L above the subject's alanine aminotransferase level prior to administration of the therapeutically effective amount of trifluridine. In some aspects, after the therapeutically effective amount of trifluridine is administered for at least 14 days, the subject's alanine aminotransferase level does not rise more than 15 U/L, more than 10 U/L, or more than 5 U/L above the subject's alanine aminotransferase level prior to administration of the therapeutically effective amount of trifluridine.

In some aspects, after the therapeutically effective amount of trifluridine is administered for at least 28 days, the subject's alanine aminotransferase level does not rise more than about 20 U/L above the subject's alanine aminotransferase level prior to administration of the therapeutically effective amount of trifluridine. In some aspects, after the therapeutically effective amount of trifluridine is administered for at least 28 days, the subject's alanine aminotransferase level does not rise more than about 15 U/L, more than about 10 U/L, or more than about 5 U/L above the subject's alanine aminotransferase level prior to administration of the therapeutically effective amount of trifluridine.

In some aspects, after the therapeutically effective amount of trifluridine is administered for at least 28 days, the subject's alanine aminotransferase level does not rise more than 20 U/L above the subject's alanine aminotransferase level prior to administration of the therapeutically effective amount of trifluridine. In some aspects, after the therapeutically effective amount of trifluridine is administered for at least 28 days, the subject's alanine aminotransferase level does not rise more than 15 U/L, more than 10 U/L, or more than 5 U/L above the subject's alanine aminotransferase level prior to administration of the therapeutically effective amount of trifluridine.

In some aspects, after the therapeutically effective amount of trifluridine is administered for at least 2 months, the subject's alanine aminotransferase level does not rise more than about 20 U/L above the subject's alanine aminotransferase level prior to administration of the therapeutically effective amount of trifluridine. In some aspects, after the therapeutically effective amount of trifluridine is administered for at least 2 months, the subject's alanine aminotransferase level does not rise more than about 15 U/L, more than about 10 U/L, or more than about 5 U/L above the subject's alanine aminotransferase level prior to administration of the therapeutically effective amount of trifluridine.

In some aspects, after the therapeutically effective amount of trifluridine is administered for at least 2 months, the subject's alanine aminotransferase level does not rise more than 20 U/L above the subject's alanine aminotransferase level prior to administration of the therapeutically effective amount of trifluridine. In some aspects, after the therapeutically effective amount of trifluridine is administered for at least 2 months, the subject's alanine aminotransferase level does not rise more than 15 U/L, more than 10 U/L, or more than 5 U/L above the subject's alanine aminotransferase level prior to administration of the therapeutically effective amount of trifluridine.

In some aspects, after the therapeutically effective amount of trifluridine is administered for at least 3 months, the subject's alanine aminotransferase level does not rise more than about 20 U/L above the subject's alanine aminotransferase level prior to administration of the therapeutically effective amount of trifluridine. In some aspects, after the therapeutically effective amount of trifluridine is administered for at least 3 months, the subject's alanine aminotransferase level does not rise more than about 15 U/L, more than about 10 U/L, or more than about 5 U/L above the subject's alanine aminotransferase level prior to administration of the therapeutically effective amount of trifluridine.

In some aspects, after the therapeutically effective amount of trifluridine is administered for at least 3 months, the subject's alanine aminotransferase level does not rise more than 20 U/L above the subject's alanine aminotransferase level prior to administration of the therapeutically effective amount of trifluridine. In some aspects, after the therapeutically effective amount of trifluridine is administered for at least 3 months, the subject's alanine aminotransferase level does not rise more than 15 U/L, more than 10 U/L, or more than 5 U/L above the subject's alanine aminotransferase level prior to administration of the therapeutically effective amount of trifluridine.

In some aspects, after the therapeutically effective amount of trifluridine is administered for at least 6 months, the subject's alanine aminotransferase level does not rise more than about 20 U/L above the subject's alanine aminotransferase level prior to administration of the therapeutically effective amount of trifluridine. In some aspects, after the therapeutically effective amount of trifluridine is administered for at least 6 months, the subject's alanine aminotransferase level does not rise more than about 15 U/L, more than about 10 U/L, or more than about 5 U/L above the subject's alanine aminotransferase level prior to administration of the therapeutically effective amount of trifluridine.

In some aspects, after the therapeutically effective amount of trifluridine is administered for at least 6 months, the subject's alanine aminotransferase level does not rise more than 20 U/L above the subject's alanine aminotransferase level prior to administration of the therapeutically effective amount of trifluridine. In some aspects, after the therapeutically effective amount of trifluridine is administered for at least 6 months, the subject's alanine aminotransferase level does not rise more than 15 U/L, more than 10 U/L, or more than 5 U/L above the subject's alanine aminotransferase level prior to administration of the therapeutically effective amount of trifluridine.

In some aspects, after the therapeutically effective amount of trifluridine is administered for at least 1 year, the subject's alanine aminotransferase level does not rise more than about 20 U/L above the subject's alanine aminotransferase level prior to administration of the therapeutically effective amount of trifluridine. In some aspects, after the therapeutically effective amount of trifluridine is administered for at least 1 year, the subject's alanine aminotransferase level does not rise more than about 15 U/L, more than about 10 U/L, or more than about 5 U/L above the subject's alanine aminotransferase level prior to administration of the therapeutically effective amount of trifluridine.

In some aspects, after the therapeutically effective amount of trifluridine is administered for at least 1 year, the subject's alanine aminotransferase level does not rise more than 20 U/L above the subject's alanine aminotransferase level prior to administration of the therapeutically effective amount of trifluridine. In some aspects, after the therapeutically effective amount of trifluridine is administered for at least 1 year, the subject's alanine aminotransferase level does not rise more than 15 U/L, more than 10 U/L, or more than 5 U/L above the subject's alanine aminotransferase level prior to administration of the therapeutically effective amount of trifluridine.

In some aspects, after the therapeutically effective amount of trifluridine is administered for at least 2 years, the subject's alanine aminotransferase level does not rise more than about 20 U/L above the subject's alanine aminotransferase level prior to administration of the therapeutically effective amount of trifluridine. In some aspects, after the therapeutically effective amount of trifluridine is administered for at least 2 years, the subject's alanine aminotransferase level does not rise more than about 15 U/L, more than about 10 U/L, or more than about 5 U/L above the subject's alanine aminotransferase level prior to administration of the therapeutically effective amount of trifluridine.

In some aspects, after the therapeutically effective amount of trifluridine is administered for at least 2 years, the subject's alanine aminotransferase level does not rise more than 20 U/L above the subject's alanine aminotransferase level prior to administration of the therapeutically effective amount of trifluridine. In some aspects, after the therapeutically effective amount of trifluridine is administered for at least 2 years, the subject's alanine aminotransferase level does not rise more than 15 U/L, more than 10 U/L, or more than 5 U/L above the subject's alanine aminotransferase level prior to administration of the therapeutically effective amount of trifluridine.

In some aspects, after the therapeutically effective amount of trifluridine is administered for at least 1 day, the subject's aspartate aminotransferase level does not rise more than about 20% above the subject's aspartate aminotransferase level prior to administration of the therapeutically effective amount of trifluridine. In some aspects, after the therapeutically effective amount of trifluridine is administered for at least 1 day, the subject's aspartate aminotransferase level does not rise more than about 15%, more than about 10%, or more than about 5% above the subject's aspartate aminotransferase level prior to administration of the therapeutically effective amount of trifluridine.

In some aspects, after the therapeutically effective amount of trifluridine is administered for at least 1 day, the subject's aspartate aminotransferase level does not rise more than 20% above the subject's aspartate aminotransferase level prior to administration of the therapeutically effective amount of trifluridine. In some aspects, after the therapeutically effective amount of trifluridine is administered for at least 1 day, the subject's aspartate aminotransferase level does not rise more than 15%, more than 10%, or more than 5% above the subject's aspartate aminotransferase level prior to administration of the therapeutically effective amount of trifluridine.

In some aspects, after the therapeutically effective amount of trifluridine is administered for at least 3 days, the subject's aspartate aminotransferase level does not rise more than about 20% above the subject's aspartate aminotransferase level prior to administration of the therapeutically effective amount of trifluridine. In some aspects, after the therapeutically effective amount of trifluridine is administered for at least 3 days, the subject's aspartate aminotransferase level does not rise more than about 15%, more than about 10%, or more than about 5% above the subject's aspartate aminotransferase level prior to administration of the therapeutically effective amount of trifluridine.

In some aspects, after the therapeutically effective amount of trifluridine is administered for at least 3 days, the subject's aspartate aminotransferase level does not rise more than 20% above the subject's aspartate aminotransferase level prior to administration of the therapeutically effective amount of trifluridine. In some aspects, after the therapeutically effective amount of trifluridine is administered for at least 3 days, the subject's aspartate aminotransferase level does not rise more than 15%, more than 10%, or more than 5% above the subject's aspartate aminotransferase level prior to administration of the therapeutically effective amount of trifluridine.

In some aspects, after the therapeutically effective amount of trifluridine is administered for at least 7 days, the subject's aspartate aminotransferase level does not rise more than about 20% above the subject's aspartate aminotransferase level prior to administration of the therapeutically effective amount of trifluridine. In some aspects, after the therapeutically effective amount of trifluridine is administered for at least 7 days, the subject's aspartate aminotransferase level does not rise more than about 15%, more than about 10%, or more than about 5% above the subject's aspartate aminotransferase level prior to administration of the therapeutically effective amount of trifluridine.

In some aspects, after the therapeutically effective amount of trifluridine is administered for at least 7 days, the subject's aspartate aminotransferase level does not rise more than 20% above the subject's aspartate aminotransferase level prior to administration of the therapeutically effective amount of trifluridine. In some aspects, after the therapeutically effective amount of trifluridine is administered for at least 7 days, the subject's aspartate aminotransferase level does not rise more than 15%, more than 10%, or more than 5% above the subject's aspartate aminotransferase level prior to administration of the therapeutically effective amount of trifluridine.

In some aspects, after the therapeutically effective amount of trifluridine is administered for at least 14 days, the subject's aspartate aminotransferase level does not rise more than about 20% above the subject's aspartate aminotransferase level prior to administration of the therapeutically effective amount of trifluridine. In some aspects, after the therapeutically effective amount of trifluridine is administered for at least 14 days, the subject's aspartate aminotransferase level does not rise more than about 15%, more than about 10%, or more than about 5% above the subject's aspartate aminotransferase level prior to administration of the therapeutically effective amount of trifluridine.

In some aspects, after the therapeutically effective amount of trifluridine is administered for at least 14 days, the subject's aspartate aminotransferase level does not rise more than 20% above the subject's aspartate aminotransferase level prior to administration of the therapeutically effective amount of trifluridine. In some aspects, after the therapeutically effective amount of trifluridine is administered for at least 14 days, the subject's aspartate aminotransferase level does not rise more than 15%, more than 10%, or more than 5% above the subject's aspartate aminotransferase level prior to administration of the therapeutically effective amount of trifluridine.

In some aspects, after the therapeutically effective amount of trifluridine is administered for at least 28 days, the subject's aspartate aminotransferase level does not rise more than about 20% above the subject's aspartate aminotransferase level prior to administration of the therapeutically effective amount of trifluridine. In some aspects, after the therapeutically effective amount of trifluridine is administered for at least 28 days, the subject's aspartate aminotransferase level does not rise more than about 15%, more than about 10%, or more than about 5% above the subject's aspartate aminotransferase level prior to administration of the therapeutically effective amount of trifluridine.

In some aspects, after the therapeutically effective amount of trifluridine is administered for at least 28 days, the subject's aspartate aminotransferase level does not rise more than 20% above the subject's aspartate aminotransferase level prior to administration of the therapeutically effective amount of trifluridine. In some aspects, after the therapeutically effective amount of trifluridine is administered for at least 28 days, the subject's aspartate aminotransferase level does not rise more than 15%, more than 10%, or more than 5% above the subject's aspartate aminotransferase level prior to administration of the therapeutically effective amount of trifluridine.

In some aspects, after the therapeutically effective amount of trifluridine is administered for at least 2 months, the subject's aspartate aminotransferase level does not rise more than about 20% above the subject's aspartate aminotransferase level prior to administration of the therapeutically effective amount of trifluridine. In some aspects, after the therapeutically effective amount of trifluridine is administered for at least 2 months, the subject's aspartate aminotransferase level does not rise more than about 15%, more than about 10%, or more than about 5% above the subject's aspartate aminotransferase level prior to administration of the therapeutically effective amount of trifluridine.

In some aspects, after the therapeutically effective amount of trifluridine is administered for at least 2 months, the subject's aspartate aminotransferase level does not rise more than 20% above the subject's aspartate aminotransferase level prior to administration of the therapeutically effective amount of trifluridine. In some aspects, after the therapeutically effective amount of trifluridine is administered for at least 2 months, the subject's aspartate aminotransferase level does not rise more than 15%, more than 10%, or more than 5% above the subject's aspartate aminotransferase level prior to administration of the therapeutically effective amount of trifluridine.

In some aspects, after the therapeutically effective amount of trifluridine is administered for at least 3 months, the subject's aspartate aminotransferase level does not rise more than about 20% above the subject's aspartate aminotransferase level prior to administration of the therapeutically effective amount of trifluridine. In some aspects, after the therapeutically effective amount of trifluridine is administered for at least 3 months, the subject's aspartate aminotransferase level does not rise more than about 15%, more than about 10%, or more than about 5% above the subject's aspartate aminotransferase level prior to administration of the therapeutically effective amount of trifluridine.

In some aspects, after the therapeutically effective amount of trifluridine is administered for at least 3 months, the subject's aspartate aminotransferase level does not rise more than 20% above the subject's aspartate aminotransferase level prior to administration of the therapeutically effective amount of trifluridine. In some aspects, after the therapeutically effective amount of trifluridine is administered for at least 3 months, the subject's aspartate aminotransferase level does not rise more than 15%, more than 10%, or more than 5% above the subject's aspartate aminotransferase level prior to administration of the therapeutically effective amount of trifluridine.

In some aspects, after the therapeutically effective amount of trifluridine is administered for at least 6 months, the subject's aspartate aminotransferase level does not rise more than about 20% above the subject's aspartate aminotransferase level prior to administration of the therapeutically effective amount of trifluridine. In some aspects, after the therapeutically effective amount of trifluridine is administered for at least 6 months, the subject's aspartate aminotransferase level does not rise more than about 15%, more than about 10%, or more than about 5% above the subject's aspartate aminotransferase level prior to administration of the therapeutically effective amount of trifluridine.

In some aspects, after the therapeutically effective amount of trifluridine is administered for at least 6 months, the subject's aspartate aminotransferase level does not rise more than 20% above the subject's aspartate aminotransferase level prior to administration of the therapeutically effective amount of trifluridine. In some aspects, after the therapeutically effective amount of trifluridine is administered for at least 6 months, the subject's aspartate aminotransferase level does not rise more than 15%, more than 10%, or more than 5% above the subject's aspartate aminotransferase level prior to administration of the therapeutically effective amount of trifluridine.

In some aspects, after the therapeutically effective amount of trifluridine is administered for at least 1 year, the subject's aspartate aminotransferase level does not rise more than about 20% above the subject's aspartate aminotransferase level prior to administration of the therapeutically effective amount of trifluridine. In some aspects, after the therapeutically effective amount of trifluridine is administered for at least 1 year, the subject's aspartate aminotransferase level does not rise more than about 15%, more than about 10%, or more than about 5% above the subject's aspartate aminotransferase level prior to administration of the therapeutically effective amount of trifluridine.

In some aspects, after the therapeutically effective amount of trifluridine is administered for at least 1 year, the subject's aspartate aminotransferase level does not rise more than 20% above the subject's aspartate aminotransferase level prior to administration of the therapeutically effective amount of trifluridine. In some aspects, after the therapeutically effective amount of trifluridine is administered for at least 1 year, the subject's aspartate aminotransferase level does not rise more than 15%, more than 10%, or more than 5% above the subject's aspartate aminotransferase level prior to administration of the therapeutically effective amount of trifluridine.

In some aspects, after the therapeutically effective amount of trifluridine is administered for at least 2 years, the subject's aspartate aminotransferase level does not rise more than about 20% above the subject's aspartate aminotransferase level prior to administration of the therapeutically effective amount of trifluridine. In some aspects, after the therapeutically effective amount of trifluridine is administered for at least 2 years, the subject's aspartate aminotransferase level does not rise more than about 15%, more than about 10%, or more than about 5% above the subject's aspartate aminotransferase level prior to administration of the therapeutically effective amount of trifluridine.

In some aspects, after the therapeutically effective amount of trifluridine is administered for at least 2 years, the subject's aspartate aminotransferase level does not rise more than 20% above the subject's aspartate aminotransferase level prior to administration of the therapeutically effective amount of trifluridine. In some aspects, after the therapeutically effective amount of trifluridine is administered for at least 2 years, the subject's aspartate aminotransferase level does not rise more than 15%, more than 10%, or more than 5% above the subject's aspartate aminotransferase level prior to administration of the therapeutically effective amount of trifluridine.

In some aspects, after the therapeutically effective amount of trifluridine is administered for at least 1 day, the subject's aspartate aminotransferase level does not rise more than about 20 U/L above the subject's aspartate aminotransferase level prior to administration of the therapeutically effective amount of trifluridine. In some aspects, after the therapeutically effective amount of trifluridine is administered for at least 1 day, the subject's aspartate aminotransferase level does not rise more than about 15 U/L, more than about 10 U/L, or more than about 5 U/L above the subject's aspartate aminotransferase level prior to administration of the therapeutically effective amount of trifluridine.

In some aspects, after the therapeutically effective amount of trifluridine is administered for at least 1 day, the subject's aspartate aminotransferase level does not rise more than 20 U/L above the subject's aspartate aminotransferase level prior to administration of the therapeutically effective amount of trifluridine. In some aspects, after the therapeutically effective amount of trifluridine is administered for at least 1 day, the subject's aspartate aminotransferase level does not rise more than 15 U/L, more than 10 U/L, or more than 5 U/L above the subject's aspartate aminotransferase level prior to administration of the therapeutically effective amount of trifluridine.

In some aspects, after the therapeutically effective amount of trifluridine is administered for at least 3 days, the subject's aspartate aminotransferase level does not rise more than about 20 U/L above the subject's aspartate aminotransferase level prior to administration of the therapeutically effective amount of trifluridine. In some aspects, after the therapeutically effective amount of trifluridine is administered for at least 3 days, the subject's aspartate aminotransferase level does not rise more than about 15 U/L, more than about 10 U/L, or more than about 5 U/L above the subject's aspartate aminotransferase level prior to administration of the therapeutically effective amount of trifluridine.

In some aspects, after the therapeutically effective amount of trifluridine is administered for at least 3 days, the subject's aspartate aminotransferase level does not rise more than 20 U/L above the subject's aspartate aminotransferase level prior to administration of the therapeutically effective amount of trifluridine. In some aspects, after the therapeutically effective amount of trifluridine is administered for at least 3 days, the subject's aspartate aminotransferase level does not rise more than 15 U/L, more than 10 U/L, or more than 5 U/L above the subject's aspartate aminotransferase level prior to administration of the therapeutically effective amount of trifluridine.

In some aspects, after the therapeutically effective amount of trifluridine is administered for at least 7 days, the subject's aspartate aminotransferase level does not rise more than about 20 U/L above the subject's aspartate aminotransferase level prior to administration of the therapeutically effective amount of trifluridine. In some aspects, after the therapeutically effective amount of trifluridine is administered for at least 7 days, the subject's aspartate aminotransferase level does not rise more than about 15 U/L, more than about 10 U/L, or more than about 5 U/L above the subject's aspartate aminotransferase level prior to administration of the therapeutically effective amount of trifluridine.

In some aspects, after the therapeutically effective amount of trifluridine is administered for at least 7 days, the subject's aspartate aminotransferase level does not rise more than 20 U/L above the subject's aspartate aminotransferase level prior to administration of the therapeutically effective amount of trifluridine. In some aspects, after the therapeutically effective amount of trifluridine is administered for at least 7 days, the subject's aspartate aminotransferase level does not rise more than 15 U/L, more than 10 U/L, or more than 5 U/L above the subject's aspartate aminotransferase level prior to administration of the therapeutically effective amount of trifluridine.

In some aspects, after the therapeutically effective amount of trifluridine is administered for at least 14 days, the subject's aspartate aminotransferase level does not rise more than about 20 U/L above the subject's aspartate aminotransferase level prior to administration of the therapeutically effective amount of trifluridine. In some aspects, after the therapeutically effective amount of trifluridine is administered for at least 14 days, the subject's aspartate aminotransferase level does not rise more than about 15 U/L, more than about 10 U/L, or more than about 5 U/L above the subject's aspartate aminotransferase level prior to administration of the therapeutically effective amount of trifluridine.

In some aspects, after the therapeutically effective amount of trifluridine is administered for at least 14 days, the subject's aspartate aminotransferase level does not rise more than 20 U/L above the subject's aspartate aminotransferase level prior to administration of the therapeutically effective amount of trifluridine. In some aspects, after the therapeutically effective amount of trifluridine is administered for at least 14 days, the subject's aspartate aminotransferase level does not rise more than 15 U/L, more than 10 U/L, or more than 5 U/L above the subject's aspartate aminotransferase level prior to administration of the therapeutically effective amount of trifluridine.

In some aspects, after the therapeutically effective amount of trifluridine is administered for at least 28 days, the subject's aspartate aminotransferase level does not rise more than about 20 U/L above the subject's aspartate aminotransferase level prior to administration of the therapeutically effective amount of trifluridine. In some aspects, after the therapeutically effective amount of trifluridine is administered for at least 28 days, the subject's aspartate aminotransferase level does not rise more than about 15 U/L, more than about 10 U/L, or more than about 5 U/L above the subject's aspartate aminotransferase level prior to administration of the therapeutically effective amount of trifluridine.

In some aspects, after the therapeutically effective amount of trifluridine is administered for at least 28 days, the subject's aspartate aminotransferase level does not rise more than 20 U/L above the subject's aspartate aminotransferase level prior to administration of the therapeutically effective amount of trifluridine. In some aspects, after the therapeutically effective amount of trifluridine is administered for at least 28 days, the subject's aspartate aminotransferase level does not rise more than 15 U/L, more than 10 U/L, or more than 5 U/L above the subject's aspartate aminotransferase level prior to administration of the therapeutically effective amount of trifluridine.

In some aspects, after the therapeutically effective amount of trifluridine is administered for at least 2 months, the subject's aspartate aminotransferase level does not rise more than about 20 U/L above the subject's aspartate aminotransferase level prior to administration of the therapeutically effective amount of trifluridine. In some aspects, after the therapeutically effective amount of trifluridine is administered for at least 2 months, the subject's aspartate aminotransferase level does not rise more than about 15 U/L, more than about 10 U/L, or more than about 5 U/L above the subject's aspartate aminotransferase level prior to administration of the therapeutically effective amount of trifluridine.

In some aspects, after the therapeutically effective amount of trifluridine is administered for at least 2 months, the subject's aspartate aminotransferase level does not rise more than 20 U/L above the subject's aspartate aminotransferase level prior to administration of the therapeutically effective amount of trifluridine. In some aspects, after the therapeutically effective amount of trifluridine is administered for at least 2 months, the subject's aspartate aminotransferase level does not rise more than 15 U/L, more than 10 U/L, or more than 5 U/L above the subject's aspartate aminotransferase level prior to administration of the therapeutically effective amount of trifluridine.

In some aspects, after the therapeutically effective amount of trifluridine is administered for at least 3 months, the subject's aspartate aminotransferase level does not rise more than about 20 U/L above the subject's aspartate aminotransferase level prior to administration of the therapeutically effective amount of trifluridine. In some aspects, after the therapeutically effective amount of trifluridine is administered for at least 3 months, the subject's aspartate aminotransferase level does not rise more than about 15 U/L, more than about 10 U/L, or more than about 5 U/L above the subject's aspartate aminotransferase level prior to administration of the therapeutically effective amount of trifluridine.

In some aspects, after the therapeutically effective amount of trifluridine is administered for at least 3 months, the subject's aspartate aminotransferase level does not rise more than 20 U/L above the subject's aspartate aminotransferase level prior to administration of the therapeutically effective amount of trifluridine. In some aspects, after the therapeutically effective amount of trifluridine is administered for at least 3 months, the subject's aspartate aminotransferase level does not rise more than 15 U/L, more than 10 U/L, or more than 5 U/L above the subject's aspartate aminotransferase level prior to administration of the therapeutically effective amount of trifluridine.

In some aspects, after the therapeutically effective amount of trifluridine is administered for at least 6 months, the subject's aspartate aminotransferase level does not rise more than about 20 U/L above the subject's aspartate aminotransferase level prior to administration of the therapeutically effective amount of trifluridine. In some aspects, after the therapeutically effective amount of trifluridine is administered for at least 6 months, the subject's aspartate aminotransferase level does not rise more than about 15 U/L, more than about 10 U/L, or more than about 5 U/L above the subject's aspartate aminotransferase level prior to administration of the therapeutically effective amount of trifluridine.

In some aspects, after the therapeutically effective amount of trifluridine is administered for at least 6 months, the subject's aspartate aminotransferase level does not rise more than 20 U/L above the subject's aspartate aminotransferase level prior to administration of the therapeutically effective amount of trifluridine. In some aspects, after the therapeutically effective amount of trifluridine is administered for at least 6 months, the subject's aspartate aminotransferase level does not rise more than 15 U/L, more than 10 U/L, or more than 5 U/L above the subject's aspartate aminotransferase level prior to administration of the therapeutically effective amount of trifluridine.

In some aspects, after the therapeutically effective amount of trifluridine is administered for at least 1 year, the subject's aspartate aminotransferase level does not rise more than about 20 U/L above the subject's aspartate aminotransferase level prior to administration of the therapeutically effective amount of trifluridine. In some aspects, after the therapeutically effective amount of trifluridine is administered for at least 1 year, the subject's aspartate aminotransferase level does not rise more than about 15 U/L, more than about 10 U/L, or more than about 5 U/L above the subject's aspartate aminotransferase level prior to administration of the therapeutically effective amount of trifluridine.

In some aspects, after the therapeutically effective amount of trifluridine is administered for at least 1 year, the subject's aspartate aminotransferase level does not rise more than 20 U/L above the subject's aspartate aminotransferase level prior to administration of the therapeutically effective amount of trifluridine. In some aspects, after the therapeutically effective amount of trifluridine is administered for at least 1 year, the subject's aspartate aminotransferase level does not rise more than 15 U/L, more than 10 U/L, or more than 5 U/L above the subject's aspartate aminotransferase level prior to administration of the therapeutically effective amount of trifluridine.

In some aspects, after the therapeutically effective amount of trifluridine is administered for at least 2 years, the subject's aspartate aminotransferase level does not rise more than about 20 U/L above the subject's aspartate aminotransferase level prior to administration of the therapeutically effective amount of trifluridine. In some aspects, after the therapeutically effective amount of trifluridine is administered for at least 2 years, the subject's aspartate aminotransferase level does not rise more than about 15 U/L, more than about 10 U/L, or more than about 5 U/L above the subject's aspartate aminotransferase level prior to administration of the therapeutically effective amount of trifluridine. In some aspects, after the therapeutically effective amount of trifluridine is administered for at least 2 years, the subject's aspartate aminotransferase level does not rise more than 20 U/L above the subject's aspartate aminotransferase level prior to administration of the therapeutically effective amount of trifluridine. In some aspects, after the therapeutically effective amount of trifluridine is administered for at least 2 years, the subject's aspartate aminotransferase level does not rise more than 15 U/L, more than 10 U/L, or more than 5 U/L above the subject's aspartate aminotransferase level prior to administration of the therapeutically effective amount of trifluridine.

In some aspects, after the therapeutically effective amount of trifluridine is administered for at least 1 day, the subject's alkaline phosphatase level does not rise more than about 20% above the subject's alkaline phosphatase level prior to administration of the therapeutically effective amount of trifluridine. In some aspects, after the therapeutically effective amount of trifluridine is administered for at least 1 day, the subject's alkaline phosphatase level does not rise more than about 15%, more than about 10%, or more than about 5% above the subject's alkaline phosphatase level prior to administration of the therapeutically effective amount of trifluridine.

In some aspects, after the therapeutically effective amount of trifluridine is administered for at least 1 day, the subject's alkaline phosphatase level does not rise more than 20% above the subject's alkaline phosphatase level prior to administration of the therapeutically effective amount of trifluridine. In some aspects, after the therapeutically effective amount of trifluridine is administered for at least 1 day, the subject's alkaline phosphatase level does not rise more than 15%, more than 10%, or more than 5% above the subject's alkaline phosphatase level prior to administration of the therapeutically effective amount of trifluridine.

In some aspects, after the therapeutically effective amount of trifluridine is administered for at least 3 days, the subject's alkaline phosphatase level does not rise more than about 20% above the subject's alkaline phosphatase level prior to administration of the therapeutically effective amount of trifluridine. In some aspects, after the therapeutically effective amount of trifluridine is administered for at least 3 days, the subject's alkaline phosphatase level does not rise more than about 15%, more than about 10%, or more than about 5% above the subject's alkaline phosphatase level prior to administration of the therapeutically effective amount of trifluridine.

In some aspects, after the therapeutically effective amount of trifluridine is administered for at least 3 days, the subject's alkaline phosphatase level does not rise more than 20% above the subject's alkaline phosphatase level prior to administration of the therapeutically effective amount of trifluridine. In some aspects, after the therapeutically effective amount of trifluridine is administered for at least 3 days, the subject's alkaline phosphatase level does not rise more than 15%, more than 10%, or more than 5% above the subject's alkaline phosphatase level prior to administration of the therapeutically effective amount of trifluridine.

In some aspects, after the therapeutically effective amount of trifluridine is administered for at least 7 days, the subject's alkaline phosphatase level does not rise more than about 20% above the subject's alkaline phosphatase level prior to administration of the therapeutically effective amount of trifluridine. In some aspects, after the therapeutically effective amount of trifluridine is administered for at least 7 days, the subject's alkaline phosphatase level does not rise more than about 15%, more than about 10%, or more than about 5% above the subject's alkaline phosphatase level prior to administration of the therapeutically effective amount of trifluridine.

In some aspects, after the therapeutically effective amount of trifluridine is administered for at least 7 days, the subject's alkaline phosphatase level does not rise more than 20% above the subject's alkaline phosphatase level prior to administration of the therapeutically effective amount of trifluridine. In some aspects, after the therapeutically effective amount of trifluridine is administered for at least 7 days, the subject's alkaline phosphatase level does not rise more than 15%, more than 10%, or more than 5% above the subject's alkaline phosphatase level prior to administration of the therapeutically effective amount of trifluridine.

In some aspects, after the therapeutically effective amount of trifluridine is administered for at least 14 days, the subject's alkaline phosphatase level does not rise more than about 20% above the subject's alkaline phosphatase level prior to administration of the therapeutically effective amount of trifluridine. In some aspects, after the therapeutically effective amount of trifluridine is administered for at least 14 days, the subject's alkaline phosphatase level does not rise more than about 15%, more than about 10%, or more than about 5% above the subject's alkaline phosphatase level prior to administration of the therapeutically effective amount of trifluridine.

In some aspects, after the therapeutically effective amount of trifluridine is administered for at least 14 days, the subject's alkaline phosphatase level does not rise more than 20% above the subject's alkaline phosphatase level prior to administration of the therapeutically effective amount of trifluridine. In some aspects, after the therapeutically effective amount of trifluridine is administered for at least 14 days, the subject's alkaline phosphatase level does not rise more than 15%, more than 10%, or more than 5% above the subject's alkaline phosphatase level prior to administration of the therapeutically effective amount of trifluridine.

In some aspects, after the therapeutically effective amount of trifluridine is administered for at least 28 days, the subject's alkaline phosphatase level does not rise more than about 20% above the subject's alkaline phosphatase level prior to administration of the therapeutically effective amount of trifluridine. In some aspects, after the therapeutically effective amount of trifluridine is administered for at least 28 days, the subject's alkaline phosphatase level does not rise more than about 15%, more than about 10%, or more than about 5% above the subject's alkaline phosphatase level prior to administration of the therapeutically effective amount of trifluridine.

In some aspects, after the therapeutically effective amount of trifluridine is administered for at least 28 days, the subject's alkaline phosphatase level does not rise more than 20% above the subject's alkaline phosphatase level prior to administration of the therapeutically effective amount of trifluridine. In some aspects, after the therapeutically effective amount of trifluridine is administered for at least 28 days, the subject's alkaline phosphatase level does not rise more than 15%, more than 10%, or more than 5% above the subject's alkaline phosphatase level prior to administration of the therapeutically effective amount of trifluridine.

In some aspects, after the therapeutically effective amount of trifluridine is administered for at least 2 months, the subject's alkaline phosphatase level does not rise more than about 20% above the subject's alkaline phosphatase level prior to administration of the therapeutically effective amount of trifluridine. In some aspects, after the therapeutically effective amount of trifluridine is administered for at least 2 months, the subject's alkaline phosphatase level does not rise more than about 15%, more than about 10%, or more than about 5% above the subject's alkaline phosphatase level prior to administration of the therapeutically effective amount of trifluridine.

In some aspects, after the therapeutically effective amount of trifluridine is administered for at least 2 months, the subject's alkaline phosphatase level does not rise more than 20% above the subject's alkaline phosphatase level prior to administration of the therapeutically effective amount of trifluridine. In some aspects, after the therapeutically effective amount of trifluridine is administered for at least 2 months, the subject's alkaline phosphatase level does not rise more than 15%, more than 10%, or more than 5% above the subject's alkaline phosphatase level prior to administration of the therapeutically effective amount of trifluridine.

In some aspects, after the therapeutically effective amount of trifluridine is administered for at least 3 months, the subject's alkaline phosphatase level does not rise more than about 20% above the subject's alkaline phosphatase level prior to administration of the therapeutically effective amount of trifluridine. In some aspects, after the therapeutically effective amount of trifluridine is administered for at least 3 months, the subject's alkaline phosphatase level does not rise more than about 15%, more than about 10%, or more than about 5% above the subject's alkaline phosphatase level prior to administration of the therapeutically effective amount of trifluridine.

In some aspects, after the therapeutically effective amount of trifluridine is administered for at least 3 months, the subject's alkaline phosphatase level does not rise more than 20% above the subject's alkaline phosphatase level prior to administration of the therapeutically effective amount of trifluridine. In some aspects, after the therapeutically effective amount of trifluridine is administered for at least 3 months, the subject's alkaline phosphatase level does not rise more than 15%, more than 10%, or more than 5% above the subject's alkaline phosphatase level prior to administration of the therapeutically effective amount of trifluridine.

In some aspects, after the therapeutically effective amount of trifluridine is administered for at least 6 months, the subject's alkaline phosphatase level does not rise more than about 20% above the subject's alkaline phosphatase level prior to administration of the therapeutically effective amount of trifluridine. In some aspects, after the therapeutically effective amount of trifluridine is administered for at least 6 months, the subject's alkaline phosphatase level does not rise more than about 15%, more than about 10%, or more than about 5% above the subject's alkaline phosphatase level prior to administration of the therapeutically effective amount of trifluridine.

In some aspects, after the therapeutically effective amount of trifluridine is administered for at least 6 months, the subject's alkaline phosphatase level does not rise more than 20% above the subject's alkaline phosphatase level prior to administration of the therapeutically effective amount of trifluridine. In some aspects, after the therapeutically effective amount of trifluridine is administered for at least 6 months, the subject's alkaline phosphatase level does not rise more than 15%, more than 10%, or more than 5% above the subject's alkaline phosphatase level prior to administration of the therapeutically effective amount of trifluridine.

In some aspects, after the therapeutically effective amount of trifluridine is administered for at least 1 year, the subject's alkaline phosphatase level does not rise more than about 20% above the subject's alkaline phosphatase level prior to administration of the therapeutically effective amount of trifluridine. In some aspects, after the therapeutically effective amount of trifluridine is administered for at least 1 year, the subject's alkaline phosphatase level does not rise more than about 15%, more than about 10%, or more than about 5% above the subject's alkaline phosphatase level prior to administration of the therapeutically effective amount of trifluridine.

In some aspects, after the therapeutically effective amount of trifluridine is administered for at least 1 year, the subject's alkaline phosphatase level does not rise more than 20% above the subject's alkaline phosphatase level prior to administration of the therapeutically effective amount of trifluridine. In some aspects, after the therapeutically effective amount of trifluridine is administered for at least 1 year, the subject's alkaline phosphatase level does not rise more than 15%, more than 10%, or more than 5% above the subject's alkaline phosphatase level prior to administration of the therapeutically effective amount of trifluridine.

In some aspects, after the therapeutically effective amount of trifluridine is administered for at least 2 years, the subject's alkaline phosphatase level does not rise more than about 20% above the subject's alkaline phosphatase level prior to administration of the therapeutically effective amount of trifluridine. In some aspects, after the therapeutically effective amount of trifluridine is administered for at least 2 years, the subject's alkaline phosphatase level does not rise more than about 15%, more than about 10%, or more than about 5% above the subject's alkaline phosphatase level prior to administration of the therapeutically effective amount of trifluridine.

In some aspects, after the therapeutically effective amount of trifluridine is administered for at least 2 years, the subject's alkaline phosphatase level does not rise more than 20% above the subject's alkaline phosphatase level prior to administration of the therapeutically effective amount of trifluridine. In some aspects, after the therapeutically effective amount of trifluridine is administered for at least 2 years, the subject's alkaline phosphatase level does not rise more than 15%, more than 10%, or more than 5% above the subject's alkaline phosphatase level prior to administration of the therapeutically effective amount of trifluridine.

In some aspects, after the therapeutically effective amount of trifluridine is administered for at least 1 day, the subject's alkaline phosphatase level does not rise more than about 50 IU/L above the subject's alkaline phosphatase level prior to administration of the therapeutically effective amount of trifluridine. In some aspects, after the therapeutically effective amount of trifluridine is administered for at least 1 day, the subject's alkaline phosphatase level does not rise more than about 40 IU/L, more than about 30 IU/L, more than about 20 IU/L, or more than about 10 IU/L above the subject's alkaline phosphatase level prior to administration of the therapeutically effective amount of trifluridine.

In some aspects, after the therapeutically effective amount of trifluridine is administered for at least 1 day, the subject's alkaline phosphatase level does not rise more than 50 IU/L above the subject's alkaline phosphatase level prior to administration of the therapeutically effective amount of trifluridine. In some aspects, after the therapeutically effective amount of trifluridine is administered for at least 1 day, the subject's alkaline phosphatase level does not rise more than 40 IU/L, more than 30 IU/L, more than 20 IU/L, or more than 10 IU/L above the subject's alkaline phosphatase level prior to administration of the therapeutically effective amount of trifluridine.

In some aspects, after the therapeutically effective amount of trifluridine is administered for at least 3 days, the subject's alkaline phosphatase level does not rise more than about 50 IU/L above the subject's alkaline phosphatase level prior to administration of the therapeutically effective amount of trifluridine. In some aspects, after the therapeutically effective amount of trifluridine is administered for at least 3 days, the subject's alkaline phosphatase level does not rise more than about 40 IU/L, more than about 30 IU/L, more than about 20 IU/L, or more than about 10 IU/L above the subject's alkaline phosphatase level prior to administration of the therapeutically effective amount of trifluridine.

In some aspects, after the therapeutically effective amount of trifluridine is administered for at least 3 days, the subject's alkaline phosphatase level does not rise more than 50 IU/L above the subject's alkaline phosphatase level prior to administration of the therapeutically effective amount of trifluridine. In some aspects, after the therapeutically effective amount of trifluridine is administered for at least 3 days, the subject's alkaline phosphatase level does not rise more than 40 IU/L, more than 30 IU/L, more than 20 IU/L, or more than 10 IU/L above the subject's alkaline phosphatase level prior to administration of the therapeutically effective amount of trifluridine.

In some aspects, after the therapeutically effective amount of trifluridine is administered for at least 7 days, the subject's alkaline phosphatase level does not rise more than about 50 IU/L above the subject's alkaline phosphatase level prior to administration of the therapeutically effective amount of trifluridine. In some aspects, after the therapeutically effective amount of trifluridine is administered for at least 7 days, the subject's alkaline phosphatase level does not rise more than about 40 IU/L, more than about 30 IU/L, more than about 20 IU/L, or more than about 10 IU/L above the subject's alkaline phosphatase level prior to administration of the therapeutically effective amount of trifluridine.

In some aspects, after the therapeutically effective amount of trifluridine is administered for at least 7 days, the subject's alkaline phosphatase level does not rise more than 50 IU/L above the subject's alkaline phosphatase level prior to administration of the therapeutically effective amount of trifluridine. In some aspects, after the therapeutically effective amount of trifluridine is administered for at least 7 days, the subject's alkaline phosphatase level does not rise more than 40 IU/L, more than 30 IU/L, more than 20 IU/L, or more than 10 IU/L above the subject's alkaline phosphatase level prior to administration of the therapeutically effective amount of trifluridine.

In some aspects, after the therapeutically effective amount of trifluridine is administered for at least 14 days, the subject's alkaline phosphatase level does not rise more than about 50 IU/L above the subject's alkaline phosphatase level prior to administration of the therapeutically effective amount of trifluridine. In some aspects, after the therapeutically effective amount of trifluridine is administered for at least 14 days, the subject's alkaline phosphatase level does not rise more than about 40 IU/L, more than about 30 IU/L, more than about 20 IU/L, or more than about 10 IU/L above the subject's alkaline phosphatase level prior to administration of the therapeutically effective amount of trifluridine.

In some aspects, after the therapeutically effective amount of trifluridine is administered for at least 14 days, the subject's alkaline phosphatase level does not rise more than 50 IU/L above the subject's alkaline phosphatase level prior to administration of the therapeutically effective amount of trifluridine. In some aspects, after the therapeutically effective amount of trifluridine is administered for at least 14 days, the subject's alkaline phosphatase level does not rise more than 40 IU/L, more than 30 IU/L, more than 20 IU/L, or more than 10 IU/L above the subject's alkaline phosphatase level prior to administration of the therapeutically effective amount of trifluridine.

In some aspects, after the therapeutically effective amount of trifluridine is administered for at least 28 days, the subject's alkaline phosphatase level does not rise more than about 50 IU/L above the subject's alkaline phosphatase level prior to administration of the therapeutically effective amount of trifluridine. In some aspects, after the therapeutically effective amount of trifluridine is administered for at least 28 days, the subject's alkaline phosphatase level does not rise more than about 40 IU/L, more than about 30 IU/L, more than about 20 IU/L, or more than about 10 IU/L above the subject's alkaline phosphatase level prior to administration of the therapeutically effective amount of trifluridine.

In some aspects, after the therapeutically effective amount of trifluridine is administered for at least 28 days, the subject's alkaline phosphatase level does not rise more than 50 IU/L above the subject's alkaline phosphatase level prior to administration of the therapeutically effective amount of trifluridine. In some aspects, after the therapeutically effective amount of trifluridine is administered for at least 28 days, the subject's alkaline phosphatase level does not rise more than 40 IU/L, more than 30 IU/L, more than 20 IU/L, or more than 10 IU/L above the subject's alkaline phosphatase level prior to administration of the therapeutically effective amount of trifluridine.

In some aspects, after the therapeutically effective amount of trifluridine is administered for at least 2 months, the subject's alkaline phosphatase level does not rise more than about 50 IU/L above the subject's alkaline phosphatase level prior to administration of the therapeutically effective amount of trifluridine. In some aspects, after the therapeutically effective amount of trifluridine is administered for at least 2 months, the subject's alkaline phosphatase level does not rise more than about 40 IU/L, more than about 30 IU/L, more than about 20 IU/L, or more than about 10 IU/L above the subject's alkaline phosphatase level prior to administration of the therapeutically effective amount of trifluridine.

In some aspects, after the therapeutically effective amount of trifluridine is administered for at least 2 months, the subject's alkaline phosphatase level does not rise more than 50 IU/L above the subject's alkaline phosphatase level prior to administration of the therapeutically effective amount of trifluridine. In some aspects, after the therapeutically effective amount of trifluridine is administered for at least 2 months, the subject's alkaline phosphatase level does not rise more than 40 IU/L, more than 30 IU/L, more than 20 IU/L, or more than 10 IU/L above the subject's alkaline phosphatase level prior to administration of the therapeutically effective amount of trifluridine.

In some aspects, after the therapeutically effective amount of trifluridine is administered for at least 3 months, the subject's alkaline phosphatase level does not rise more than about 50 IU/L above the subject's alkaline phosphatase level prior to administration of the therapeutically effective amount of trifluridine. In some aspects, after the therapeutically effective amount of trifluridine is administered for at least 3 months, the subject's alkaline phosphatase level does not rise more than about 40 IU/L, more than about 30 IU/L, more than about 20 IU/L, or more than about 10 IU/L above the subject's alkaline phosphatase level prior to administration of the therapeutically effective amount of trifluridine.

In some aspects, after the therapeutically effective amount of trifluridine is administered for at least 3 months, the subject's alkaline phosphatase level does not rise more than 50 IU/L above the subject's alkaline phosphatase level prior to administration of the therapeutically effective amount of trifluridine. In some aspects, after the therapeutically effective amount of trifluridine is administered for at least 3 months, the subject's alkaline phosphatase level does not rise more than 40 IU/L, more than 30 IU/L, more than 20 IU/L, or more than 10 IU/L above the subject's alkaline phosphatase level prior to administration of the therapeutically effective amount of trifluridine.

In some aspects, after the therapeutically effective amount of trifluridine is administered for at least 6 months, the subject's alkaline phosphatase level does not rise more than about 50 IU/L above the subject's alkaline phosphatase level prior to administration of the therapeutically effective amount of trifluridine. In some aspects, after the therapeutically effective amount of trifluridine is administered for at least 6 months, the subject's alkaline phosphatase level does not rise more than about 40 IU/L, more than about 30 IU/L, more than about 20 IU/L, or more than about 10 IU/L above the subject's alkaline phosphatase level prior to administration of the therapeutically effective amount of trifluridine.

In some aspects, after the therapeutically effective amount of trifluridine is administered for at least 6 months, the subject's alkaline phosphatase level does not rise more than 50 IU/L above the subject's alkaline phosphatase level prior to administration of the therapeutically effective amount of trifluridine. In some aspects, after the therapeutically effective amount of trifluridine is administered for at least 6 months, the subject's alkaline phosphatase level does not rise more than 40 IU/L, more than 30 IU/L, more than 20 IU/L, or more than 10 IU/L above the subject's alkaline phosphatase level prior to administration of the therapeutically effective amount of trifluridine.

In some aspects, after the therapeutically effective amount of trifluridine is administered for at least 1 year, the subject's alkaline phosphatase level does not rise more than about 50 IU/L above the subject's alkaline phosphatase level prior to administration of the therapeutically effective amount of trifluridine. In some aspects, after the therapeutically effective amount of trifluridine is administered for at least 1 year, the subject's alkaline phosphatase level does not rise more than about 40 IU/L, more than about 30 IU/L, more than about 20 IU/L, or more than about 10 IU/L above the subject's alkaline phosphatase level prior to administration of the therapeutically effective amount of trifluridine.

In some aspects, after the therapeutically effective amount of trifluridine is administered for at least 1 year, the subject's alkaline phosphatase level does not rise more than 50 IU/L above the subject's alkaline phosphatase level prior to administration of the therapeutically effective amount of trifluridine. In some aspects, after the therapeutically effective amount of trifluridine is administered for at least 1 year, the subject's alkaline phosphatase level does not rise more than 40 IU/L, more than 30 IU/L, more than 20 IU/L, or more than 10 IU/L above the subject's alkaline phosphatase level prior to administration of the therapeutically effective amount of trifluridine.

In some aspects, after the therapeutically effective amount of trifluridine is administered for at least 2 years, the subject's alkaline phosphatase level does not rise more than about 50 IU/L above the subject's alkaline phosphatase level prior to administration of the therapeutically effective amount of trifluridine. In some aspects, after the therapeutically effective amount of trifluridine is administered for at least 2 years, the subject's alkaline phosphatase level does not rise more than about 40 IU/L, more than about 30 IU/L, more than about 20 IU/L, or more than about 10 IU/L above the subject's alkaline phosphatase level prior to administration of the therapeutically effective amount of trifluridine.

In some aspects, after the therapeutically effective amount of trifluridine is administered for at least 2 years, the subject's alkaline phosphatase level does not rise more than 50 IU/L above the subject's alkaline phosphatase level prior to administration of the therapeutically effective amount of trifluridine. In some aspects, after the therapeutically effective amount of trifluridine is administered for at least 2 years, the subject's alkaline phosphatase level does not rise more than 40 IU/L, more than 30 IU/L, more than 20 IU/L, or more than 10 IU/L above the subject's alkaline phosphatase level prior to administration of the therapeutically effective amount of trifluridine.

In some aspects, after the therapeutically effective amount of trifluridine is administered for at least 1 day, the subject's total bilirubin level does not rise more than about 20% above the subject's total bilirubin level prior to administration of the therapeutically effective amount of trifluridine. In some aspects, after the therapeutically effective amount of trifluridine is administered for at least 1 day, the subject's total bilirubin level does not rise more than about 15%, more than about 10%, or more than about 5% above the subject's total bilirubin level prior to administration of the therapeutically effective amount of trifluridine.

In some aspects, after the therapeutically effective amount of trifluridine is administered for at least 1 day, the subject's total bilirubin level does not rise more than 20% above the subject's total bilirubin level prior to administration of the therapeutically effective amount of trifluridine. In some aspects, after the therapeutically effective amount of trifluridine is administered for at least 1 day, the subject's total bilirubin level does not rise more than 15%, more than 10%, or more than 5% above the subject's total bilirubin level prior to administration of the therapeutically effective amount of trifluridine.

In some aspects, after the therapeutically effective amount of trifluridine is administered for at least 3 days, the subject's total bilirubin level does not rise more than about 20% above the subject's total bilirubin level prior to administration of the therapeutically effective amount of trifluridine. In some aspects, after the therapeutically effective amount of trifluridine is administered for at least 3 days, the subject's total bilirubin level does not rise more than about 15%, more than about 10%, or more than about 5% above the subject's total bilirubin level prior to administration of the therapeutically effective amount of trifluridine.

In some aspects, after the therapeutically effective amount of trifluridine is administered for at least 3 days, the subject's total bilirubin level does not rise more than 20% above the subject's total bilirubin level prior to administration of the therapeutically effective amount of trifluridine. In some aspects, after the therapeutically effective amount of trifluridine is administered for at least 3 days, the subject's total bilirubin level does not rise more than 15%, more than 10%, or more than 5% above the subject's total bilirubin level prior to administration of the therapeutically effective amount of trifluridine.

In some aspects, after the therapeutically effective amount of trifluridine is administered for at least 7 days, the subject's total bilirubin level does not rise more than about 20% above the subject's total bilirubin level prior to administration of the therapeutically effective amount of trifluridine. In some aspects, after the therapeutically effective amount of trifluridine is administered for at least 7 days, the subject's total bilirubin level does not rise more than about 15%, more than about 10%, or more than about 5% above the subject's total bilirubin level prior to administration of the therapeutically effective amount of trifluridine.

In some aspects, after the therapeutically effective amount of trifluridine is administered for at least 7 days, the subject's total bilirubin level does not rise more than 20% above the subject's total bilirubin level prior to administration of the therapeutically effective amount of trifluridine. In some aspects, after the therapeutically effective amount of trifluridine is administered for at least 7 days, the subject's total bilirubin level does not rise more than 15%, more than 10%, or more than 5% above the subject's total bilirubin level prior to administration of the therapeutically effective amount of trifluridine.

In some aspects, after the therapeutically effective amount of trifluridine is administered for at least 14 days, the subject's total bilirubin level does not rise more than about 20% above the subject's total bilirubin level prior to administration of the therapeutically effective amount of trifluridine. In some aspects, after the therapeutically effective amount of trifluridine is administered for at least 14 days, the subject's total bilirubin level does not rise more than about 15%, more than about 10%, or more than about 5% above the subject's total bilirubin level prior to administration of the therapeutically effective amount of trifluridine.

In some aspects, after the therapeutically effective amount of trifluridine is administered for at least 14 days, the subject's total bilirubin level does not rise more than 20% above the subject's total bilirubin level prior to administration of the therapeutically effective amount of trifluridine. In some aspects, after the therapeutically effective amount of trifluridine is administered for at least 14 days, the subject's total bilirubin level does not rise more than 15%, more than 10%, or more than 5% above the subject's total bilirubin level prior to administration of the therapeutically effective amount of trifluridine.

In some aspects, after the therapeutically effective amount of trifluridine is administered for at least 28 days, the subject's total bilirubin level does not rise more than about 20% above the subject's total bilirubin level prior to administration of the therapeutically effective amount of trifluridine. In some aspects, after the therapeutically effective amount of trifluridine is administered for at least 28 days, the subject's total bilirubin level does not rise more than about 15%, more than about 10%, or more than about 5% above the subject's total bilirubin level prior to administration of the therapeutically effective amount of trifluridine.

In some aspects, after the therapeutically effective amount of trifluridine is administered for at least 28 days, the subject's total bilirubin level does not rise more than 20% above the subject's total bilirubin level prior to administration of the therapeutically effective amount of trifluridine. In some aspects, after the therapeutically effective amount of trifluridine is administered for at least 28 days, the subject's total bilirubin level does not rise more than 15%, more than 10%, or more than 5% above the subject's total bilirubin level prior to administration of the therapeutically effective amount of trifluridine.

In some aspects, after the therapeutically effective amount of trifluridine is administered for at least 2 months, the subject's total bilirubin level does not rise more than about 20% above the subject's total bilirubin level prior to administration of the therapeutically effective amount of trifluridine. In some aspects, after the therapeutically effective amount of trifluridine is administered for at least 2 months, the subject's total bilirubin level does not rise more than about 15%, more than about 10%, or more than about 5% above the subject's total bilirubin level prior to administration of the therapeutically effective amount of trifluridine.

In some aspects, after the therapeutically effective amount of trifluridine is administered for at least 2 months, the subject's total bilirubin level does not rise more than 20% above the subject's total bilirubin level prior to administration of the therapeutically effective amount of trifluridine. In some aspects, after the therapeutically effective amount of trifluridine is administered for at least 2 months, the subject's total bilirubin level does not rise more than 15%, more than 10%, or more than 5% above the subject's total bilirubin level prior to administration of the therapeutically effective amount of trifluridine.

In some aspects, after the therapeutically effective amount of trifluridine is administered for at least 3 months, the subject's total bilirubin level does not rise more than about 20% above the subject's total bilirubin level prior to administration of the therapeutically effective amount of trifluridine. In some aspects, after the therapeutically effective amount of trifluridine is administered for at least 3 months, the subject's total bilirubin level does not rise more than about 15%, more than about 10%, or more than about 5% above the subject's total bilirubin level prior to administration of the therapeutically effective amount of trifluridine.

In some aspects, after the therapeutically effective amount of trifluridine is administered for at least 3 months, the subject's total bilirubin level does not rise more than 20% above the subject's total bilirubin level prior to administration of the therapeutically effective amount of trifluridine. In some aspects, after the therapeutically effective amount of trifluridine is administered for at least 3 months, the subject's total bilirubin level does not rise more than 15%, more than 10%, or more than 5% above the subject's total bilirubin level prior to administration of the therapeutically effective amount of trifluridine.

In some aspects, after the therapeutically effective amount of trifluridine is administered for at least 6 months, the subject's total bilirubin level does not rise more than about 20% above the subject's total bilirubin level prior to administration of the therapeutically effective amount of trifluridine. In some aspects, after the therapeutically effective amount of trifluridine is administered for at least 6 months, the subject's total bilirubin level does not rise more than about 15%, more than about 10%, or more than about 5% above the subject's total bilirubin level prior to administration of the therapeutically effective amount of trifluridine.

In some aspects, after the therapeutically effective amount of trifluridine is administered for at least 6 months, the subject's total bilirubin level does not rise more than 20% above the subject's total bilirubin level prior to administration of the therapeutically effective amount of trifluridine. In some aspects, after the therapeutically effective amount of trifluridine is administered for at least 6 months, the subject's total bilirubin level does not rise more than 15%, more than 10%, or more than 5% above the subject's total bilirubin level prior to administration of the therapeutically effective amount of trifluridine.

In some aspects, after the therapeutically effective amount of trifluridine is administered for at least 1 year, the subject's total bilirubin level does not rise more than about 20% above the subject's total bilirubin level prior to administration of the therapeutically effective amount of trifluridine. In some aspects, after the therapeutically effective amount of trifluridine is administered for at least 1 year, the subject's total bilirubin level does not rise more than about 15%, more than about 10%, or more than about 5% above the subject's total bilirubin level prior to administration of the therapeutically effective amount of trifluridine.

In some aspects, after the therapeutically effective amount of trifluridine is administered for at least 1 year, the subject's total bilirubin level does not rise more than 20% above the subject's total bilirubin level prior to administration of the therapeutically effective amount of trifluridine. In some aspects, after the therapeutically effective amount of trifluridine is administered for at least 1 year, the subject's total bilirubin level does not rise more than 15%, more than 10%, or more than 5% above the subject's total bilirubin level prior to administration of the therapeutically effective amount of trifluridine.

In some aspects, after the therapeutically effective amount of trifluridine is administered for at least 2 years, the subject's total bilirubin level does not rise more than about 20% above the subject's total bilirubin level prior to administration of the therapeutically effective amount of trifluridine. In some aspects, after the therapeutically effective amount of trifluridine is administered for at least 2 years, the subject's total bilirubin level does not rise more than about 15%, more than about 10%, or more than about 5% above the subject's total bilirubin level prior to administration of the therapeutically effective amount of trifluridine.

In some aspects, after the therapeutically effective amount of trifluridine is administered for at least 2 years the subject's total bilirubin level does not rise more than 20% above the subject's total bilirubin level prior to administration of the therapeutically effective amount of trifluridine. In some aspects, after the therapeutically effective amount of trifluridine is administered for at least 2 years, the subject's total bilirubin level does not rise more than 15%, more than 10%, or more than 5% above the subject's total bilirubin level prior to administration of the therapeutically effective amount of trifluridine.

In some aspects, after the therapeutically effective amount of trifluridine is administered for at least 1 day, the subject's total bilirubin level does not rise more than about 1 mg/dL above the subject's total bilirubin level prior to administration of the therapeutically effective amount of trifluridine. In some aspects, after the therapeutically effective amount of trifluridine is administered for at least 1 day, the subject's total bilirubin level does not rise more than about 0.8 mg/dL, more than about 0.5 mg/dL, or more than about 0.3 mg/dL above the subject's total bilirubin level prior to administration of the therapeutically effective amount of trifluridine.

In some aspects, after the therapeutically effective amount of trifluridine is administered for at least 1 day, the subject's total bilirubin level does not rise more than 1 mg/dL above the subject's total bilirubin level prior to administration of the therapeutically effective amount of trifluridine. In some aspects, after the therapeutically effective amount of trifluridine is administered for at least 1 day, the subject's total bilirubin level does not rise more than 0.8 mg/dL, more than 0.5 mg/dL, or more than 0.3 mg/dL above the subject's total bilirubin level prior to administration of the therapeutically effective amount of trifluridine.

In some aspects, after the therapeutically effective amount of trifluridine is administered for at least 3 days, the subject's total bilirubin level does not rise more than about 1 mg/dL above the subject's total bilirubin level prior to administration of the therapeutically effective amount of trifluridine. In some aspects, after the therapeutically effective amount of trifluridine is administered for at least 3 days, the subject's total bilirubin level does not rise more than about 0.8 mg/dL, more than about 0.5 mg/dL, or more than about 0.3 mg/dL above the subject's total bilirubin level prior to administration of the therapeutically effective amount of trifluridine.

In some aspects, after the therapeutically effective amount of trifluridine is administered for at least 3 days, the subject's total bilirubin level does not rise more than 1 mg/dL above the subject's total bilirubin level prior to administration of the therapeutically effective amount of trifluridine. In some aspects, after the therapeutically effective amount of trifluridine is administered for at least 3 days, the subject's total bilirubin level does not rise more than 0.8 mg/dL, more than 0.5 mg/dL, or more than 0.3 mg/dL above the subject's total bilirubin level prior to administration of the therapeutically effective amount of trifluridine.

In some aspects, after the therapeutically effective amount of trifluridine is administered for at least 7 days, the subject's total bilirubin level does not rise more than about 1 mg/dL above the subject's total bilirubin level prior to administration of the therapeutically effective amount of trifluridine. In some aspects, after the therapeutically effective amount of trifluridine is administered for at least 7 days, the subject's total bilirubin level does not rise more than about 0.8 mg/dL, more than about 0.5 mg/dL, or more than about 0.3 mg/dL above the subject's total bilirubin level prior to administration of the therapeutically effective amount of trifluridine.

In some aspects, after the therapeutically effective amount of trifluridine is administered for at least 7 days, the subject's total bilirubin level does not rise more than 1 mg/dL above the subject's total bilirubin level prior to administration of the therapeutically effective amount of trifluridine. In some aspects, after the therapeutically effective amount of trifluridine is administered for at least 7 days, the subject's total bilirubin level does not rise more than 0.8 mg/dL, more than 0.5 mg/dL, or more than 0.3 mg/dL above the subject's total bilirubin level prior to administration of the therapeutically effective amount of trifluridine.

In some aspects, after the therapeutically effective amount of trifluridine is administered for at least 14 days, the subject's total bilirubin level does not rise more than about 1 mg/dL above the subject's total bilirubin level prior to administration of the therapeutically effective amount of trifluridine. In some aspects, after the therapeutically effective amount of trifluridine is administered for at least 14 days, the subject's total bilirubin level does not rise more than about 0.8 mg/dL, more than about 0.5 mg/dL, or more than about 0.3 mg/dL above the subject's total bilirubin level prior to administration of the therapeutically effective amount of trifluridine.

In some aspects, after the therapeutically effective amount of trifluridine is administered for at least 14 days, the subject's total bilirubin level does not rise more than 1 mg/dL above the subject's total bilirubin level prior to administration of the therapeutically effective amount of trifluridine. In some aspects, after the therapeutically effective amount of trifluridine is administered for at least 14 days, the subject's total bilirubin level does not rise more than 0.8 mg/dL, more than 0.5 mg/dL, or more than 0.3 mg/dL above the subject's total bilirubin level prior to administration of the therapeutically effective amount of trifluridine.

In some aspects, after the therapeutically effective amount of trifluridine is administered for at least 28 days, the subject's total bilirubin level does not rise more than about 1 mg/dL above the subject's total bilirubin level prior to administration of the therapeutically effective amount of trifluridine. In some aspects, after the therapeutically effective amount of trifluridine is administered for at least 28 days, the subject's total bilirubin level does not rise more than about 0.8 mg/dL, more than about 0.5 mg/dL, or more than about 0.3 mg/dL above the subject's total bilirubin level prior to administration of the therapeutically effective amount of trifluridine.

In some aspects, after the therapeutically effective amount of trifluridine is administered for at least 28 days, the subject's total bilirubin level does not rise more than 1 mg/dL above the subject's total bilirubin level prior to administration of the therapeutically effective amount of trifluridine. In some aspects, after the therapeutically effective amount of trifluridine is administered for at least 28 days, the subject's total bilirubin level does not rise more than 0.8 mg/dL, more than 0.5 mg/dL, or more than 0.3 mg/dL above the subject's total bilirubin level prior to administration of the therapeutically effective amount of trifluridine.

In some aspects, after the therapeutically effective amount of trifluridine is administered for at least 2 months, the subject's total bilirubin level does not rise more than about 1 mg/dL above the subject's total bilirubin level prior to administration of the therapeutically effective amount of trifluridine. In some aspects, after the therapeutically effective amount of trifluridine is administered for at least 2 months, the subject's total bilirubin level does not rise more than about 0.8 mg/dL, more than about 0.5 mg/dL, or more than about 0.3 mg/dL above the subject's total bilirubin level prior to administration of the therapeutically effective amount of trifluridine.

In some aspects, after the therapeutically effective amount of trifluridine is administered for at least 2 months, the subject's total bilirubin level does not rise more than 1 mg/dL above the subject's total bilirubin level prior to administration of the therapeutically effective amount of trifluridine. In some aspects, after the therapeutically effective amount of trifluridine is administered for at least 2 months, the subject's total bilirubin level does not rise more than 0.8 mg/dL, more than 0.5 mg/dL, or more than 0.3 mg/dL above the subject's total bilirubin level prior to administration of the therapeutically effective amount of trifluridine.

In some aspects, after the therapeutically effective amount of trifluridine is administered for at least 3 months, the subject's total bilirubin level does not rise more than about 1 mg/dL above the subject's total bilirubin level prior to administration of the therapeutically effective amount of trifluridine. In some aspects, after the therapeutically effective amount of trifluridine is administered for at least 3 months, the subject's total bilirubin level does not rise more than about 0.8 mg/dL, more than about 0.5 mg/dL, or more than about 0.3 mg/dL above the subject's total bilirubin level prior to administration of the therapeutically effective amount of trifluridine.

In some aspects, after the therapeutically effective amount of trifluridine is administered for at least 3 months, the subject's total bilirubin level does not rise more than 1 mg/dL above the subject's total bilirubin level prior to administration of the therapeutically effective amount of trifluridine. In some aspects, after the therapeutically effective amount of trifluridine is administered for at least 3 months, the subject's total bilirubin level does not rise more than 0.8 mg/dL, more than 0.5 mg/dL, or more than 0.3 mg/dL above the subject's total bilirubin level prior to administration of the therapeutically effective amount of trifluridine.

In some aspects, after the therapeutically effective amount of trifluridine is administered for at least 6 months, the subject's total bilirubin level does not rise more than about 1 mg/dL above the subject's total bilirubin level prior to administration of the therapeutically effective amount of trifluridine. In some aspects, after the therapeutically effective amount of trifluridine is administered for at least 6 months, the subject's total bilirubin level does not rise more than about 0.8 mg/dL, more than about 0.5 mg/dL, or more than about 0.3 mg/dL above the subject's total bilirubin level prior to administration of the therapeutically effective amount of trifluridine.

In some aspects, after the therapeutically effective amount of trifluridine is administered for at least 6 months, the subject's total bilirubin level does not rise more than 1 mg/dL above the subject's total bilirubin level prior to administration of the therapeutically effective amount of trifluridine. In some aspects, after the therapeutically effective amount of trifluridine is administered for at least 6 months, the subject's total bilirubin level does not rise more than 0.8 mg/dL, more than 0.5 mg/dL, or more than 0.3 mg/dL above the subject's total bilirubin level prior to administration of the therapeutically effective amount of trifluridine.

In some aspects, after the therapeutically effective amount of trifluridine is administered for at least 1 year, the subject's total bilirubin level does not rise more than about 1 mg/dL above the subject's total bilirubin level prior to administration of the therapeutically effective amount of trifluridine. In some aspects, after the therapeutically effective amount of trifluridine is administered for at least 1 year, the subject's total bilirubin level does not rise more than about 0.8 mg/dL, more than about 0.5 mg/dL, or more than about 0.3 mg/dL above the subject's total bilirubin level prior to administration of the therapeutically effective amount of trifluridine.

In some aspects, after the therapeutically effective amount of trifluridine is administered for at least 1 year, the subject's total bilirubin level does not rise more than 1 mg/dL above the subject's total bilirubin level prior to administration of the therapeutically effective amount of trifluridine. In some aspects, after the therapeutically effective amount of trifluridine is administered for at least 1 year, the subject's total bilirubin level does not rise more than 0.8 mg/dL, more than 0.5 mg/dL, or more than 0.3 mg/dL above the subject's total bilirubin level prior to administration of the therapeutically effective amount of trifluridine.

In some aspects, after the therapeutically effective amount of trifluridine is administered for at least 2 years, the subject's total bilirubin level does not rise more than about 1 mg/dL above the subject's total bilirubin level prior to administration of the therapeutically effective amount of trifluridine. In some aspects, after the therapeutically effective amount of trifluridine is administered for at least 2 years, the subject's total bilirubin level does not rise more than about 0.8 mg/dL, more than about 0.5 mg/dL, or more than about 0.3 mg/dL above the subject's total bilirubin level prior to administration of the therapeutically effective amount of trifluridine.

In some aspects, after the therapeutically effective amount of trifluridine is administered for at least 2 years, the subject's total bilirubin level does not rise more than 1 mg/dL above the subject's total bilirubin level prior to administration of the therapeutically effective amount of trifluridine. In some aspects, after the therapeutically effective amount of trifluridine is administered for at least 2 years, the subject's total bilirubin level does not rise more than 0.8 mg/dL, more than 0.5 mg/dL, or more than 0.3 mg/dL above the subject's total bilirubin level prior to administration of the therapeutically effective amount of trifluridine.

In some aspects, the therapeutically effective amount of trifluridine is administered in a treatment cycle. As used herein, “a treatment cycle” includes a period of treatment followed by a period of rest (no treatment) that can be repeated on a regular schedule. For example, treatment given for one week followed by three weeks of rest is one treatment cycle. By way of another example, treatment given for two weeks followed by two weeks of rest is one treatment cycle. In some aspects, the therapeutically effective amount of trifluridine is administered in more than one treatment cycle. In some aspects, the therapeutically effective amount of trifluridine is administered in two or more treatment cycles (after initial treatment, e.g., the first infusion).

In some aspects, the interval between the start of a treatment cycle and the start of the next treatment cycle is about 1 week to about 8 weeks. In some aspects, the interval between the start of a treatment cycle and the start of the next treatment cycle is about 2 to about 8 weeks. In some aspects, the interval between the start of a treatment cycle and the start of the next treatment cycle is about 3 weeks to about 8 weeks, about 4 weeks to about 8 weeks, about 6 weeks to about 8 weeks, about 1 week to about 6 weeks, about 2 weeks to about 6 weeks, about 3 weeks to about 6 weeks, about 4 weeks to about 6 weeks, about 1 week to about 4 weeks, about 2 weeks to about 4 weeks, or about 3 weeks to about 4 weeks. In some aspects, the interval between the start of a treatment cycle and the start of the next treatment cycle is about 1 week, about 2 weeks, about 3 weeks, about 4 weeks, about 5 weeks, about 6 weeks, about 7 weeks, or about 8 weeks.

In some aspects, the interval between the start of a treatment cycle and the next treatment cycle is 1 week to 8 weeks. In some aspects, the interval between the start of a treatment cycle and the start of the next treatment cycle is 2 to 8 weeks. In some aspects, the interval between the start of a treatment cycle and the start of the next treatment cycle is 3 weeks to 8 weeks, 4 weeks to 8 weeks, 6 weeks to 8 weeks, 1 week to 6 weeks, 2 weeks to 6 weeks, 3 weeks to 6 weeks, 4 weeks to 6 weeks, 1 week to 4 weeks, 2 weeks to 4 weeks, or 3 weeks to 4 weeks. In some aspects, the interval between the start of a treatment cycle and the start of the next treatment cycle is 1 week, 2 weeks, 3 weeks, 4 weeks, 5 weeks, 6 weeks, 7 weeks, or 8 weeks.

In some aspects, the interval between the start of a treatment cycle and the next treatment cycle is about 1 week to about 8 weeks. In some aspects, the interval between the start of a treatment cycle and the start of the next treatment cycle is about 1 week to about 7 weeks, about 1 to week to about 6 weeks, about 1 week to about 5 weeks, about 1 week to about 4 weeks, about 1 week to about 3 weeks, or about 1 week to about 2 weeks. In some aspects, the interval between the start of a treatment cycle and the start of the next treatment cycle is about 2 weeks to about 8 weeks, about 2 weeks to about 7 weeks, about 2 weeks to about 6 weeks, about 2 weeks to about 5 weeks, about 2 weeks to about 4 weeks, or about 2 weeks to about 3 weeks. In some aspects, the interval between the start of a treatment cycle and the start of the next treatment cycle is about 3 weeks to about 8 weeks, about 3 weeks to about 7 weeks, about 3 weeks to about 6 weeks, about 3 weeks to about 5 weeks, or about 3 weeks to about 4 weeks. In some aspects, the interval between the start of a treatment cycle and the start of the next treatment cycle is about 4 weeks to about 8 weeks, about 4 weeks to about 7 weeks, about 4 weeks to about 6 weeks, or about 4 weeks to about 5 weeks. In some aspects, the interval between the start of a treatment cycle and the start of the next treatment cycle is about 5 weeks to about 8 weeks, about 5 weeks to about 7 weeks, or about 5 weeks to about 6 weeks. In some aspects, the interval between the start of a treatment cycle and the start of the next treatment cycle is about 6 weeks to about 8 weeks, or about 6 weeks to about 7 weeks. In some aspects, the interval between the start of a treatment cycle and the start of the next treatment cycle is about 7 weeks to about 8 weeks. In some aspects, the interval between the start of a treatment cycle and the start of the next treatment cycle is about 1 week, about 2 weeks, about 3 weeks, about 4 weeks, about 5 weeks, about 6 weeks, about 7 weeks, or about 8 weeks.

In some aspects, the interval between the start of a treatment cycle and the next treatment cycle is 1 week to 8 weeks between treatments. In some aspects, the interval between the start of a treatment cycle and the start of the next treatment cycle is 1 week to 7 weeks, 1 to week to 6 weeks, 1 week to 5 weeks, 1 week to 4 weeks, 1 week to 3 weeks, or 1 week to 2 weeks. In some aspects, the interval between the start of a treatment cycle and the start of the next treatment cycle is 2 weeks to 8 weeks, 2 weeks to 7 weeks, 2 weeks to 6 weeks, 2 weeks to 5 weeks, 2 weeks to 4 weeks, or 2 weeks to 3 weeks. In some aspects, the interval between the start of a treatment cycle and the start of the next treatment cycle is 3 weeks to 8 weeks, 3 weeks to 7 weeks, 3 weeks to 6 weeks, 3 weeks to 5 weeks, or 3 weeks to 4 weeks. In some aspects, the interval between the start of a treatment cycle and the start of the next treatment cycle is 4 weeks to 8 weeks, 4 weeks to 7 weeks, 4 weeks to 6 weeks, or 4 weeks to 5 weeks. In some aspects, the interval between the start of a treatment cycle and the start of the next treatment cycle is 5 weeks to 8 weeks, 5 weeks to 7 weeks, or 5 weeks to 6 weeks. In some aspects, the interval between the start of a treatment cycle and the start of the next treatment cycle is 6 weeks to 8 weeks, or 6 weeks to 7 weeks. In some aspects, the interval between the start of a treatment cycle and the start of the next treatment cycle is 7 weeks to 8 weeks. In some aspects, the interval between the start of a treatment cycle and the start of the next treatment cycle is 1 week, 2 weeks, 3 weeks, 4 weeks, 5 weeks, 6 weeks, 7 weeks, or 8 weeks. Doses administered in each treatment cycle may be identical or different. The number of treatment cycles and the doses to be administered may be determined by the physician according to the physiological state of the patient, and the evolution of the disease. In some aspects, one or several further treatment cycles as described above are administered during an indefinite period of time.

In some aspects, one or several further treatment cycles as described above are administered over the course of about 3 years. In some aspects, one or several further treatment cycles as described above are administered over the course of about 2 years. In some aspects, one or several further treatment cycles as described above are administered over the course of about 1 year. In some aspects, one or several further treatment cycles as described above are administered over the course of about 9 months. In some aspects, one or several further treatment cycles as described above are administered over the course of about 6 months.

In some aspects, one or several further treatment cycles as described above are administered over the course of 3 years. In some aspects, one or several further treatment cycles as described above are administered over the course of 2 years. In some aspects, one or several further treatment cycles as described above are administered over the course of about 1 year. In some aspects, one or several further treatment cycles as described above are administered over the course of 9 months. In some aspects, one or several further treatment cycles as described above are administered over the course of 6 months. In some aspects, one or several further treatment cycles as described above are administered over the course of 3 months.

In some aspects, the subject has a plasma concentration of trifluridine of about 0.5 μM or less after the therapeutically effective amount of trifluridine is administered for at least 1 day. In some aspects, the subject has a plasma concentration of trifluridine of, about 0.4 μM or less, about 0.3 μM or less, about 0.2 μM or less, about 0.1 μM or less, about 0.09 μM or less, about 0.08 μM or less, about 0.07 μM or less, about 0.06 μM or less, or about 0.05 μM or less after the therapeutically effective amount of trifluridine is administered for at least 1 day.

In some aspects, the subject has a plasma concentration of trifluridine of 0.5 μM or less after the therapeutically effective amount of trifluridine is administered for at least 1 day. In some aspects, the subject has a plasma concentration of trifluridine of, 0.4 μM or less, 0.3 μM or less, 0.2 μM or less, 0.1 μM or less, 0.09 μM or less, 0.08 μM or less, 0.07 μM or less, 0.06 μM or less, or 0.05 μM or less after the therapeutically effective amount of trifluridine is administered for at least 1 day.

In some aspects, the subject has a plasma concentration of trifluridine of about 0.5 μM or less after the therapeutically effective amount of trifluridine is administered for at least 3 days. In some aspects, the subject has a plasma concentration of trifluridine of, about 0.4 μM or less, about 0.3 μM or less, about 0.2 μM or less, about 0.1 μM or less, about 0.09 μM or less, about 0.08 μM or less, about 0.07 μM or less, about 0.06 μM or less, or about 0.05 μM or less after the therapeutically effective amount of trifluridine is administered for at least 3 days.

In some aspects, the subject has a plasma concentration of trifluridine of 0.5 μM or less after the therapeutically effective amount of trifluridine is administered for at least 3 days. In some aspects, the subject has a plasma concentration of trifluridine of, 0.4 μM or less, 0.3 μM or less, 0.2 μM or less, 0.1 μM or less, 0.09 μM or less, 0.08 μM or less, 0.07 μM or less, 0.06 μM or less, or 0.05 μM or less after the therapeutically effective amount of trifluridine is administered for at least 3 days.

In some aspects, the subject has a plasma concentration of trifluridine of about 0.5 μM or less after the therapeutically effective amount of trifluridine is administered for at least 7 days. In some aspects, the subject has a plasma concentration of trifluridine of, about 0.4 μM or less, about 0.3 μM or less, about 0.2 μM or less, about 0.1 μM or less, about 0.09 μM or less, about 0.08 μM or less, about 0.07 μM or less, about 0.06 μM or less, or about 0.05 μM or less after the therapeutically effective amount of trifluridine is administered for at least 7 days.

In some aspects, the subject has a plasma concentration of trifluridine of 0.5 μM or less after the therapeutically effective amount of trifluridine is administered for at least 7 days. In some aspects, the subject has a plasma concentration of trifluridine of, 0.4 μM or less, 0.3 μM or less, 0.2 μM or less, 0.1 μM or less, 0.09 μM or less, 0.08 μM or less, 0.07 μM or less, 0.06 μM or less, or 0.05 μM or less after the therapeutically effective amount of trifluridine is administered for at least 7 days.

In some aspects, the subject has a plasma concentration of trifluridine of about 0.5 μM or less after the therapeutically effective amount of trifluridine is administered for at least 14 days. In some aspects, the subject has a plasma concentration of trifluridine of about 0.4 μM or less, about 0.3 μM or less, about 0.2 μM or less, about 0.1 μM or less, about 0.09 μM or less, about 0.08 μM or less, about 0.07 μM or less, about 0.06 μM or less, or about 0.05 μM or less after the therapeutically effective amount of trifluridine is administered for at least 14 days.

In some aspects, the subject has a plasma concentration of trifluridine of 0.5 μM or less after the therapeutically effective amount of trifluridine is administered for at least 14 days. In some aspects, the subject has a plasma concentration of trifluridine of 0.4 μM or less, 0.3 μM or less, 0.2 μM or less, 0.1 μM or less, 0.09 μM or less, 0.08 μM or less, 0.07 μM or less, 0.06 μM or less, or 0.05 μM or less after the therapeutically effective amount of trifluridine is administered for at least 14 days.

In some aspects, the subject has a plasma concentration of trifluridine of about 0.5 μM or less after the therapeutically effective amount of trifluridine is administered for at least 28 days. In some aspects, the subject has a plasma concentration of trifluridine of about 0.4 μM or less, about 0.3 μM or less, about 0.2 μM or less, about 0.1 μM or less, about 0.09 μM or less, about 0.08 μM or less, about 0.07 μM or less, about 0.06 μM or less, or about 0.05 μM or less after the therapeutically effective amount of trifluridine is administered for at least 28 days.

In some aspects, the subject has a plasma concentration of trifluridine of 0.5 μM or less after the therapeutically effective amount of trifluridine is administered for at least 28 days. In some aspects, the subject has a plasma concentration of trifluridine of 0.4 μM or less, 0.3 μM or less, 0.2 μM or less, 0.1 μM or less, 0.09 μM or less, 0.08 μM or less, 0.07 μM or less, 0.06 μM or less, or 0.05 μM or less after the therapeutically effective amount of trifluridine is administered for at least 28 days.

In some aspects, the subject has a plasma concentration of trifluridine of about 0.5 μM or less after the therapeutically effective amount of trifluridine is administered for at least 2 months. In some aspects, the subject has a plasma concentration of trifluridine of about 0.4 μM or less, about 0.3 μM or less, about 0.2 μM or less, about 0.1 μM or less, about 0.09 μM or less, about 0.08 μM or less, about 0.07 μM or less, about 0.06 μM or less, or about 0.05 μM or less after the therapeutically effective amount of trifluridine is administered for at least 2 months.

In some aspects, the subject has a plasma concentration of trifluridine of 0.5 μM or less after the therapeutically effective amount of trifluridine is administered for at least 2 months. In some aspects, the subject has a plasma concentration of trifluridine of 0.4 μM or less, 0.3 μM or less, 0.2 μM or less, 0.1 μM or less, 0.09 μM or less, 0.08 μM or less, 0.07 μM or less, 0.06 μM or less, or 0.05 μM or less after the therapeutically effective amount of trifluridine is administered for at least 2 months.

In some aspects, the subject has a plasma concentration of trifluridine of about 0.5 μM or less after the therapeutically effective amount of trifluridine is administered for at least 3 months. In some aspects, the subject has a plasma concentration of trifluridine of about 0.4 μM or less, about 0.3 μM or less, about 0.2 μM or less, about 0.1 μM or less, about 0.09 μM or less, about 0.08 μM or less, about 0.07 μM or less, about 0.06 μM or less, or about 0.05 μM or less after the therapeutically effective amount of trifluridine is administered for at least 3 months.

In some aspects, the subject has a plasma concentration of trifluridine of 0.5 μM or less after the therapeutically effective amount of trifluridine is administered for at least 3 months. In some aspects, the subject has a plasma concentration of trifluridine of 0.4 μM or less, 0.3 μM or less, 0.2 μM or less, 0.1 μM or less, 0.09 μM or less, 0.08 μM or less, 0.07 μM or less, 0.06 μM or less, or 0.05 μM or less after the therapeutically effective amount of trifluridine is administered for at least 3 months.

In some aspects, the subject has a plasma concentration of trifluridine of about 0.5 μM or less after the therapeutically effective amount of trifluridine is administered for at least 6 months. In some aspects, the subject has a plasma concentration of trifluridine of about 0.4 μM or less, about 0.3 μM or less, about 0.2 μM or less, about 0.1 μM or less, about 0.09 μM or less, about 0.08 μM or less, about 0.07 μM or less, about 0.06 μM or less, or about 0.05 μM or less after the therapeutically effective amount of trifluridine is administered for at least 6 months.

In some aspects, the subject has a plasma concentration of trifluridine of 0.5 μM or less after the therapeutically effective amount of trifluridine is administered for at least 6 months. In some aspects, the subject has a plasma concentration of trifluridine of 0.4 μM or less, 0.3 μM or less, 0.2 μM or less, 0.1 μM or less, 0.09 μM or less, 0.08 μM or less, 0.07 μM or less, 0.06 μM or less, or 0.05 μM or less after the therapeutically effective amount of trifluridine is administered for at least 6 months.

In some aspects, the subject has a plasma concentration of trifluridine of about 0.5 μM or less after the therapeutically effective amount of trifluridine is administered for at least 1 year. In some aspects, the subject has a plasma concentration of trifluridine of about 0.4 μM or less, about 0.3 μM or less, about 0.2 μM or less, about 0.1 μM or less, about 0.09 μM or less, about 0.08 μM or less, about 0.07 μM or less, about 0.06 μM or less, or about 0.05 μM or less after the therapeutically effective amount of trifluridine is administered for at least 1 year.

In some aspects, the subject has a plasma concentration of trifluridine of 0.5 μM or less after the therapeutically effective amount of trifluridine is administered for at least 1 year. In some aspects, the subject has a plasma concentration of trifluridine of 0.4 μM or less, 0.3 μM or less, 0.2 μM or less, 0.1 μM or less, 0.09 μM or less, 0.08 μM or less, 0.07 μM or less, 0.06 μM or less, or 0.05 μM or less after the therapeutically effective amount of trifluridine is administered for at least 1 year.

In some aspects, the subject has a plasma concentration of trifluridine of about 0.5 μM or less after the therapeutically effective amount of trifluridine is administered for at least 2 years. In some aspects, the subject has a plasma concentration of trifluridine of about 0.4 μM or less, about 0.3 μM or less, about 0.2 μM or less, about 0.1 μM or less, about 0.09 μM or less, about 0.08 μM or less, about 0.07 μM or less, about 0.06 μM or less, or about 0.05 μM or less after the therapeutically effective amount of trifluridine is administered for at least 2 years.

In some aspects, the subject has a plasma concentration of trifluridine of 0.5 μM or less after the therapeutically effective amount of trifluridine is administered for at least 2 years. In some aspects, the subject has a plasma concentration of trifluridine of 0.4 μM or less, 0.3 μM or less, 0.2 μM or less, 0.1 μM or less, 0.09 μM or less, 0.08 μM or less, 0.07 μM or less, 0.06 μM or less, or 0.05 μM or less after the therapeutically effective amount of trifluridine is administered for at least 2 years.

In some aspects, the method of treating liver cancer further includes administering a therapeutically effective amount of at least one additional therapeutic agent. In some aspects, the at least one additional therapeutic agent is a chemotherapeutic agent.

In some aspects, the chemotherapeutic agent is aminoglutethimide, amsacrine, anastrozole, asparaginase, bcg, bicalutamide, bleomycin, buserelin, busulfan, campothecin, capecitabine, carboplatin, carmustine, chlorambucil, cisplatin, cladribine, clodronate, colchicine, cyclophosphamide, cyproterone, cytarabine, dacarbazine, dactinomycin, daunorubicin, dienestrol, diethylstilbestrol, docetaxel, doxorubicin, epirubicin, estradiol, estramustine, etoposide, exemestane, filgrastim, fludarabine, fludrocortisone, fluorouracil, fluoxymesterone, flutamide, gemcitabine, genistein, goserelin, hydroxyurea, idarubicin, ifosfamide, imatinib, interferon, irinotecan, ironotecan, letrozole, leucovorin, leuprolide, levamisole, lomustine, mechlorethamine, medroxyprogesterone, megestrol, melphalan, mercaptopurine, mesna, methotrexate, mitomycin, mitotane, mitoxantrone, nilutamide, nocodazole, octreotide, oxaliplatin, paclitaxel, pamidronate, pentostatin, plicamycin, porfimer, procarbazine, raltitrexed, rituximab, streptozocin, suramin, tamoxifen, temozolomide, teniposide, testosterone, thioguanine, thiotepa, titanocene dichloride, topotecan, trastuzumab, tretinoin, vinblastine, vincristine, vindesine, and vinorelbine.

In some aspects, the chemotherapeutic agent is an anti-metabolite/anti-cancer agent, such as pyrimidine analogs (5-fluorouracil, floxuridine, capecitabine, gemcitabine and cytarabine) and purine analogs, folate antagonists and related inhibitors (mercaptopurine, thioguanine, pentostatin and 2-chlorodeoxyadenosine (cladribine)); antiproliferative/antimitotic agents including natural products such as vinca alkaloids (vinblastine, vincristine, and vinorelbine), microtubule disruptors such as taxane (paclitaxel, docetaxel), vincristin, vinblastin, nocodazole, epothilones and navelbine, epidipodophyllotoxins (teniposide), DNA damaging agents (actinomycin, amsacrine, anthracyclines, bleomycin, busulfan, camptothecin, carboplatin, chlorambucil, cisplatin, cyclophosphamide, cytoxan, dactinomycin, daunorubicin, docetaxel, doxorubicin, epirubicin, hexamethylmelamineoxaliplatin, iphosphamide, melphalan, merchlorethamine, mitomycin, mitoxantrone, nitrosourea, paclitaxel, plicamycin, procarbazine, teniposide, triethylenethiophosphoramide and etoposide (VP16)); antibiotics such as dactinomycin (actinomycin D), daunorubicin, doxorubicin (adriamycin), idarubicin, anthracyclines, mitoxantrone, bleomycins, plicamycin (mithramycin) and mitomycin; enzymes (L-asparaginase); antiplatelet agents; antiproliferative/antimitotic alkylating agents such as nitrogen mustards (mechlorethamine, cyclophosphamide and analogs, melphalan, chlorambucil), ethylenimines and methylmelamines (hexamethylmelamine and thiotepa), alkyl sulfonates-busulfan, nitrosoureas (carmustine (BCNU) and analogs, streptozocin), trazenes-dacarbazinine (DTIC); antiproliferative/antimitotic antimetabolites such as folic acid analogs (methotrexate); platinum coordination complexes (cisplatin, carboplatin), procarbazine, hydroxyurea, mitotane, aminoglutethimide; hormones, hormone analogs (estrogen, tamoxifen, goserelin, bicalutamide, nilutamide) and aromatase inhibitors (letrozole, anastrozole); anticoagulants (heparin, synthetic heparin salts and other inhibitors of thrombin); fibrinolytic agents (such as tissue plasminogen activator, streptokinase and urokinase), aspirin, COX-2 inhibitors, dipyridamole, ticlopidine, clopidogrel, abciximab; antimigratory agents; antisecretory agents (breveldin); immunosuppressives (cyclosporine, tacrolimus (FK-506), sirolimus (rapamycin), azathioprine, mycophenolate mofetil); anti-angiogenic compounds (TNP-470, genistein) and growth factor inhibitors (vascular endothelial growth factor (VEGF) inhibitors, fibroblast growth factor (FGF) inhibitors, epidermal growth factor (EGF) inhibitors); angiotensin receptor blocker; nitric oxide donors; anti-sense oligonucleotides; antibodies (trastuzumab); cell cycle inhibitors and differentiation inducers (tretinoin); mTOR inhibitors, topoisomerase inhibitors (doxorubicin (adriamycin), amsacrine, camptothecin, daunorubicin, dactinomycin, eniposide, epirubicin, etoposide, idarubicin, irinotecan (CPT-11) and mitoxantrone, topotecan, irinotecan), corticosteroids (cortisone, dexamethasone, hydrocortisone, methylpednisolone, prednisone, and prenisolone); growth factor signal transduction kinase inhibitors; mitochondrial dysfunction inducers and caspase activators; and chromatin disruptors.

In some aspects, the method of treating liver cancer further includes administering a therapeutically effective amount of floxuridine. In some aspects, the method of treating liver cancer further includes administering a therapeutically effective amount of fluorouracil. In some aspects, the method of treating liver cancer further includes administering a therapeutically effective amount of a thymidine phosphorylase (TP) inhibitor. In some aspects, the TP inhibitor is tipiracil.

In some aspects, the method of treating liver cancer further includes administering an effective amount of dexamethasone. In some aspects, the effective amount of dexamethasone is an amount effective to function as an anti-inflammatory agent. In some aspects, the method of treating liver cancer further includes administering an effective amount of heparin. In some aspects, the effective amount of heparin is an amount effective to prevent clotting of the device, artery, and/or vein.

In some aspects, the method of treating liver cancer further includes administering a therapeutically effective amount of capecitabine. In some aspects, administration of capecitabine is prior to administration of trifluridine. In some aspects, a treatment cycle of capecitabine is followed by administration of trifluridine or a treatment cycle of trifluridine. In some aspects, administration of trifluridine is after resistance to capecitabine is developed.

In some aspects, administration of capecitabine is after administration of trifluridine. In some aspects, a treatment cycle of trifluridine is followed by administration of capecitabine or a treatment cycle of capecitabine. In some aspects, administration of capecitabine is after resistance to trifluridine is developed.

In some aspects, the method of treating liver cancer further includes administering dexamethasone. In some aspects, the method of treating liver cancer further includes administering about 0.1 mg/ml to about 100 mg/ml dexamethasone. In some aspects, the method of treating liver cancer further includes administering about 1 mg/ml to about 75 mg/ml dexamethasone, about 1 mg/ml to about 50 mg/ml dexamethasone, about 1 mg/ml to about 25 mg/ml dexamethasone, about 1 mg/ml to about 20 mg/ml dexamethasone, about 1 mg/ml to about 15 mg/ml dexamethasone, about 5 mg/ml to about 15 mg/ml dexamethasone. In some aspects, the method of treating liver cancer can include administering about 10 mg/ml dexamethasone.

In some aspects, the method of treating liver cancer further includes administering dexamethasone. In some aspects, the method of treating liver cancer further includes administering 0.1 mg/ml to 100 mg/ml dexamethasone. In some aspects, the method of treating liver cancer further includes administering 1 mg/ml to 75 mg/ml dexamethasone, 1 mg/ml to 50 mg/ml dexamethasone, 1 mg/ml to 25 mg/ml dexamethasone, 1 mg/ml to 20 mg/ml dexamethasone, 1 mg/ml to 15 mg/ml dexamethasone, 5 mg/ml to 15 mg/ml dexamethasone. In some aspects, the method of treating liver cancer further includes administering 10 mg/ml dexamethasone.

In some aspects, the method of treating liver cancer further includes administering heparin. In some aspects, the method of treating liver cancer further includes administering about 1,000 IU/ml to about 10,000 IU/ml heparin. In some aspects, the formulation includes about 1,000 IU/ml heparin. In some aspects, the method of treating liver cancer further includes administering about 5,000 IU/ml heparin.

In some aspects, the method of treating liver cancer further includes administering heparin. In some aspects, the method of treating liver cancer further includes administering 1,000 IU/ml to 10,000 IU/ml heparin. In some aspects, the method of treating liver cancer further includes administering 1,000 IU/ml heparin. In some aspects, the method of treating liver cancer further includes administering 5,000 IU/ml heparin.

In some aspects, treatment cycles of trifluridine and capecitabine is alternated. In some aspects, trifluridine and capecitabine is co-administered.

In some aspects, at least one additional therapeutic agent is an immunotherapeutic agent. The term “immunotherapeutic agent” refers in general to any agent that produces a therapeutic effect by targeting the immune system or a component thereof. As used herein, the immunotherapeutic agent typically promotes an immune response, e.g. the agent may be an immunostimulatory agent or an inhibitor of an immunosuppressive agent (i.e. an anti-immunosuppressive agent). Immunotherapeutic agents can also include checkpoint blockers or inhibitors, chimeric antigen receptors (CARs), and adoptive T-cell therapy.

In some aspects, the method does not comprise filtering blood of the liver.

In some aspects, the method of treating liver cancer does not comprise administering a second agent. In some aspects, the method of treating liver cancer does not comprise administering a chemotherapeutic agent. In some aspects, the method of treating liver cancer does not comprise administering a thymidine phosphorylase (TP) inhibitor. In some aspects, the method of treating liver cancer does not comprise administering the thymidine phosphorylase (TP) inhibitor tipiracil.

In some aspects, the therapeutically effective amount of trifluridine is 0.01 mg/kg/day to 3 mg/kg/day continuously administered for 7 days to an adult human by hepatic arterial infusion via an internal pump In some aspects, the therapeutically effective amount of trifluridine is 0.01 mg/kg/day to 3 mg/kg/day continuously administered for 14 days to an adult human by hepatic arterial infusion via an internal pump. In some aspects, the therapeutically effective amount of trifluridine is 0.01 mg/kg/day to 3 mg/kg/day continuously administered for 28 days to an adult human by hepatic arterial infusion via an internal pump.

In some aspects, the therapeutically effective amount of trifluridine is 0.1 mg/kg/day to 2 mg/kg/day continuously administered for 7 days to an adult human by hepatic arterial infusion via an internal pump. In some aspects, the therapeutically effective amount of trifluridine is 0.1 mg/kg/day to 2 mg/kg/day continuously administered for 14 days to an adult human by hepatic arterial infusion via an internal pump. In some aspects, the therapeutically effective amount of trifluridine is 0.1 mg/kg/day to 2 mg/kg/day continuously administered for 28 days to an adult human by hepatic arterial infusion via an internal pump.

In some aspects, the therapeutically effective amount of trifluridine is 0.7 mg/kg/day to 2 mg/kg/day continuously administered for 7 days to an adult human by hepatic arterial infusion via an internal pump. In some aspects, the therapeutically effective amount of trifluridine is 0.7 mg/kg/day to 2 mg/kg/day continuously administered for 14 days to an adult human by hepatic arterial infusion via an internal pump. In some aspects, the therapeutically effective amount of trifluridine is 0.7 mg/kg/day to 2 mg/kg/day continuously administered for 28 days to an adult human by hepatic arterial infusion via an internal pump.

### Formulations

The present disclosure is also directed to formulations that contain trifluridine. In some aspects, the formulations are liquid formulations. The formulations described herein can be administered in a method of treating liver cancer in accordance with any of the above aspects.

The formulation described herein increase trifluridine solubility and trifluridine stability in an aqueous solution at body temperature. As a result, the improved formulation can increase a daily dose of trifluridine that can be given to a subject in need thereof to increase potential for efficacy.

The inventors surprisingly found that trifluridine solubility can be increased through the addition of a co-solvent compared to trifluridine solubility in currently available products or trifluridine solubility in pure aqueous solution. But the inventors found that the viscosity of the formulation increased due to the increased trifluridine concentration and the presence of the co-solvent. This increase in viscosity negatively impacted the flow rate of such a formulation through a constant flow pump for HAI. As a result, increasing the solubility of trifluridine without considering the viscosity of the formulation did not necessarily increase the daily dose of trifluridine that can be provided to a subject in need thereof through a constant flow pump for HAI.

In addition, an anticoagulant, such as heparin or fondaparinux, is traditionally mixed into a trifluridine formulation to maintain catheter patency. The addition of an anticoagulant to the trifluridine formulation also diluted the concentration of trifluridine in the drug product solution and increased solution viscosity. This presented another challenge in designing a formulation that increased the trifluridine daily dose that can be provided to a subject in need thereof through HAI compared to currently available trifluridine products. Without being bound to any particular theory, the inventors surprisingly found that the trifluridine formulations as described in the present disclosure did not appear to require an anticoagulant, such as heparin or fondaparinux. These formulations enhanced trifluridine solubility and lowered viscosity. As a result, the trifluridine formulations described herein can increase a daily dose that can be provided to a subject in need thereof by HAI (e.g., through a constant flow pump or a peristaltic pump) compared to the trifluridine products that would require heparin or fondaparinux addition.

In some aspects, the formulation comprises about 50 mg/ml to about 150 mg/ml of trifluridine. In some aspects, the formulation comprises about 50 mg/ml to about 125 mg/ml, about 50 mg/ml to about 100 mg/ml, about 50 mg/ml to about 90 mg/ml, about 50 mg/ml to about 80 mg/ml, about 50 mg/ml to about 75 mg/ml, about 50 mg/ml to about 70 mg/ml, or about 50 mg/ml to about 60 mg/ml. In some aspects, the formulation comprises about 70 mg/ml to about 150 mg/ml, about 70 mg/ml to about 125 mg/ml, about 70 mg/ml to about 100 mg/ml, about 100 mg/ml to about 150 mg/ml, about 100 mg/ml to about 125 mg/ml, or about 125 mg/ml to about 150 mg/ml. In some aspects, the formulation comprises about 50 mg/ml to about 100 mg/ml, about 60 mg/ml to about 90 mg/ml, about 65 mg/ml to about 85 mg/ml, 65 mg/ml to about 75 mg/ml, or about 70 mg/ml to about 80 mg/ml of trifluridine.

In some aspects, the formulation comprises about 50 mg/ml of trifluridine. In some aspects, the formulation comprises about 55 mg/ml, about 60 mg/ml, about 65 mg/ml, about 70 mg/ml, about 75 mg/ml, about 80 mg/ml, about 85 mg/ml, about 90 mg/ml, about 95 mg/ml, about 100 mg/ml, about 105 mg/ml, about 110 mg/ml, about 115 mg/ml, about 120 mg/ml, about 125 mg/ml, about 130 mg/ml, about 135 mg/ml, about 140 mg/ml, about 145 mg/ml, or about 150 mg/ml of trifluridine.

In some aspects, the formulation comprises 50 mg/ml to 150 mg/ml of trifluridine. In some aspects, the formulation comprises 50 mg/ml to 125 mg/ml, 50 mg/ml to 100 mg/ml, 50 mg/ml to 90 mg/ml, 50 mg/ml to 80 mg/ml, 50 mg/ml to 75 mg/ml, 50 mg/ml to 70 mg/ml, or 50 mg/ml to 60 mg/ml. In some aspects, the formulation comprises 70 mg/ml to 150 mg/ml, 70 mg/ml to 125 mg/ml, 70 mg/ml to 100 mg/ml, 100 mg/ml to 150 mg/ml, 100 mg/ml to 125 mg/ml, or 125 mg/ml to 150 mg/ml. In some aspects, the formulation comprises 50 mg/ml to 100 mg/ml, 60 mg/ml to 90 mg/ml, 65 mg/ml to 85 mg/ml, 65 mg/ml to about 75 mg/ml, or 70 mg/ml to 80 mg/ml of trifluridine.

In some aspects, the formulation comprises 50 mg/ml of trifluridine. In some aspects, the formulation comprises 55 mg/ml, 60 mg/ml, 65 mg/ml, 70 mg/ml, 75 mg/ml, 80 mg/ml, 85 mg/ml, 90 mg/ml, 95 mg/ml, 100 mg/ml, 105 mg/ml, 110 mg/ml, 115 mg/ml, 120 mg/ml, 125 mg/ml, 130 mg/ml, 135 mg/ml, 140 mg/ml, 145 mg/ml, or 150 mg/ml of trifluridine.

In some aspects, the formulation comprises a buffer. In some aspects, the buffer comprises a phosphate buffer, a borate buffer, an acetate buffer, a carbonate buffer, a maleate buffer, a gluconate buffer, a tartrate buffer, a citrate buffer, or combinations thereof. In some aspects, the buffer is selected from the group consisting of a phosphate buffer, a borate buffer, an acetate buffer, a carbonate buffer, a maleate buffer, a gluconate buffer, a tartrate buffer, a citrate buffer, or combinations thereof. In some aspects, the formulation comprises an acetate buffer. In certain aspects, the formulation comprises a citrate buffer.

In some aspects, the formulation comprises about 5 mM to about 250 mM of buffer. In some aspects, the liquid pharmaceutical formulation comprises about 10 mM to about 50 mM, about 10 mM to about 100 mM, about 10 mM to about 150 mM, or about 10 mM to about 200 mM of buffer.

In some aspects, the formulation comprises 5 mM to 250 mM of buffer. In some aspects, the formulation comprises 10 mM to 50 mM, 10 mM to 100 mM, 10 mM to 150 mM, or 10 mM to 200 mM of buffer.

In some aspects, the formulation comprises about 25 mM to about 250 mM of buffer. In some aspects, the formulation comprises about 25 mM to about 50 mM, about 25 mM to about 100 mM, about 25 mM to about 150 mM, or about 25 mM to about 200 mM of buffer.

In some aspects, the formulation comprises 25 mM to 250 mM of buffer. In some aspects, the formulation comprises 25 mM to 50 mM, 25 mM to 100 mM, 25 mM to 150 mM, or 25 mM to 200 mM of buffer.

In some aspects, the formulation comprises about 50 mM to about 250 mM of buffer. In some aspects, the formulation comprises about 50 mM to about 100 mM, about 50 mM to about 150 mM, or about 50 mM to about 200 mM of buffer.

In some aspects, the formulation comprises 50 mM to 250 mM of buffer. In some aspects, the formulation comprises 50 mM to 100 mM, 50 mM to 150 mM, or 50 mM to 200 mM of buffer.

In some aspects, the formulation comprises about 100 mM to about 250 mM of buffer. In some aspects, the formulation comprises about 100 mM to about 150 mM, or about 100 mM to about 200 mM of buffer.

In some aspects, the formulation comprises 100 mM to 250 mM of buffer. In some aspects, the formulation comprises 100 mM to 150 mM, or 100 mM to 200 mM of buffer.

In some aspects, the formulation comprises about 25 mM to about 125 mM, about 30 mM to about 120 mM, about 40 mM to about 110 mM, or about 50 mM to about 100 mM of buffer. In some aspects, the formulation comprises about 60 mM to about 90 mM, about 65 to about 85, or about 70 mM to about 80 mM of buffer.

In some aspects, the formulation comprises 25 mM to 125 mM, 30 mM to 120 mM, 40 mM to 110 mM, or 50 mM to 100 mM of buffer. In some aspects, the formulation comprises 60 mM to 90 mM, 65 to 85, or 70 mM to 80 mM of buffer.

In some aspects, the formulation comprises about 5 mM to about 250 mM of citrate buffer. In some aspects, the formulation comprises about 5 mM to about 50 mM, about 5 mM to about 100 mM, about 5 mM to about 150 mM, or about 5 mM to about 200 mM of citrate buffer.

In some aspects, the formulation comprises 5 mM to 250 mM of citrate buffer. In some aspects, the formulation comprises 5 mM to 50 mM, 5 mM to 100 mM, 5 mM to 150 mM, or 5 mM to 200 mM of citrate buffer.

In some aspects, the formulation comprises about 25 mM to about 250 mM of citrate buffer. In some aspects, the formulation comprises about 25 mM to about 50 mM, about 25 mM to about 100 mM, about 25 mM to about 150 mM, or about 25 mM to about 200 mM of citrate buffer.

In some aspects, the formulation comprises 25 mM to 250 mM of citrate buffer. In some aspects, the formulation comprises 25 mM to 50 mM, 25 mM to 100 mM, 25 mM to 150 mM, or 25 mM to 200 mM of citrate buffer.

In some aspects, the formulation comprises about 50 mM to about 250 mM of citrate buffer. In some aspects, the formulation comprises about 50 mM to about 100 mM, about 50 mM to about 150 mM, or about 50 mM to about 200 mM of citrate buffer.

In some aspects, the formulation comprises 50 mM to 250 mM of citrate buffer. In some aspects, the formulation comprises 50 mM to 100 mM, 50 mM to 150 mM, or 50 mM to 200 mM of citrate buffer.

In some aspects, the formulation comprises about 100 mM to about 250 mM of citrate buffer. In some aspects, the formulation comprises about 100 mM to about 150 mM, or about 100 mM to about 200 mM of citrate buffer.

In some aspects, the formulation comprises 100 mM to 250 mM of citrate buffer. In some aspects, the formulation comprises 100 mM to 150 mM, or 100 mM to 200 mM of citrate buffer.

In some aspects, the formulation comprises about 25 mM to about 125 mM, about 30 mM to about 120 mM, about 40 mM to about 110 mM, or about 50 mM to about 100 mM of citrate buffer. In some aspects, the formulation comprises about 60 mM to about 90 mM, about 65 to about 85, or about 70 mM to about 80 mM of citrate buffer.

In some aspects, the formulation comprises 25 mM to 125 mM, 30 mM to 120 mM, 40 mM to 110 mM, or 50 mM to 100 mM of citrate buffer. In some aspects, the formulation comprises 60 mM to 90 mM, 65 to 85, or 70 mM to 80 mM of citrate buffer.

In some aspects, the formulation comprises a solvent system comprising water and a co-solvent. In some aspects, the co-solvent comprises ethanol, propylene glycol, dimethyl sulfoxide, dimethylacetamide, N-methyl-2-pyrrolidone, or combinations thereof. In some aspects, the co-solvent is selected from the group consisting of ethanol, propylene glycol, dimethyl sulfoxide, dimethylacetamide, N-methyl-2-pyrrolidone, or combinations thereof. In some aspects, the solvent system comprises two or more co-solvents. In some aspects, the co-solvent is ethanol. In some aspects, the co-solvent is dimethyl sulfoxide. In some aspects, the co-solvent is dimethylacetamide (DMA). In some aspects, the co-solvent is N-methyl-2-pyrrolidone.

In some aspects, the solvent system comprises about 1% to about 99%, about 1% to about 90%, about 1% to about 80%, about 1% to about 70%, about 1% to about 60%, or about 1% to about 50% v/v of co-solvent. In some aspects, the solvent system comprises about 1% to about 40%, about 1% to about 35%, about 1% to about 30%, about 1% to about 25%, about 1% to about 20%, about 1% to about 15%, or about 1% to about 10% v/v of co-solvent.

In some aspects, the solvent system comprises 1% to 99%, 1% to 90%, 1% to 80%, 1% to 70%, 1% to 60%, or 1% to 50% v/v of co-solvent. In some aspects, the solvent system comprises 1% to 40%, 1% to 35%, 1% to 30%, 1% to 25%, 1% to 20%, 1% to 15%, or 1% to 10% v/v of co-solvent.

In some aspects, the solvent system comprises about 5% to about 99%, about 5% to about 90%, about 5% to about 80%, about 5% to about 70%, about 5% to about 60%, or about 5% to about 50% v/v of co-solvent. In some aspects, the solvent system comprises about 5% to about 40%, about 5% to about 35%, about 5% to about 30%, about 5% to about 25%, about 5% to about 20%, about 5% to about 15%, or about 5% to about 10% v/v of co-solvent.

In some aspects, the solvent system comprises 5% to 99%, 5% to 90%, 5% to 80%, 5% to 70%, 5% to 60%, or 5% to 50% v/v of co-solvent. In some aspects, the solvent system comprises 5% to 40%, 5% to 35%, 5% to 30%, 5% to 25%, 5% to 20%, 5% to 15%, or 5% to 10% v/v of co-solvent.

In some aspects, the solvent system comprises about 10% to about 99%, about 10% to about 90%, about 10% to about 80%, about 10% to about 70%, about 10% to about 60%, or about 10% to about 50% v/v of co-solvent. In some aspects, the solvent system comprises about 10% to about 40%, about 10% to about 35%, about 10% to about 30%, about 10% to about 25%, about 10% to about 20%, or about 10% to about 15% v/v of co-solvent.

In some aspects, the solvent system comprises 10% to 99%, 10% to 90%, 10% to 80%, 10% to 70%, 10% to 60%, or 10% to 50% v/v of co-solvent. In some aspects, the solvent system comprises 10% to 40%, 10% to 35%, 10% to 30%, 10% to 25%, 10% to 20%, or 10% to 15% v/v of co-solvent.

In some aspects, the solvent system comprises about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, or about 30% v/v of co-solvent.

In some aspects, the solvent system comprises 10%, 11%, 12%, 13%, 14%, 15%, 16%, 17%, 18%, 19%, 20%, 21%, 22%, 23%, 24%, 25%, 26%, 27%, 28%, 29%, or 30% v/v of co-solvent.

In some aspects, the solvent system comprises about 1% to about 99%, about 1% to about 90%, about 1% to about 80%, about 1% to about 70%, about 1% to about 60%, or about 1% to about 50% v/v of DMA. In some aspects, the solvent system comprises about 1% to about 40%, about 1% to about 35%, about 1% to about 30%, about 1% to about 25%, about 1% to about 20%, about 1% to about 15%, or about 1% to about 10% v/v of DMA.

In some aspects, the solvent system comprises 1% to 99%, 1% to 90%, 1% to 80%, 1% to 70%, 1% to 60%, or 1% to 50% v/v of DMA. In some aspects, the solvent system comprises 1% to 40%, 1% to 35%, 1% to 30%, 1% to 25%, 1% to 20%, 1% to 15%, or 1% to 10% v/v of DMA.

In some aspects, the solvent system comprises about 5% to about 99%, about 5% to about 90%, about 5% to about 80%, about 5% to about 70%, about 5% to about 60%, or about 5% to about 50% v/v of DMA. In some aspects, the solvent system comprises about 5% to about 40%, about 5% to about 35%, about 5% to about 30%, about 5% to about 25%, about 5% to about 20%, about 5% to about 15%, or about 5% to about 10% v/v of DMA.

In some aspects, the solvent system comprises 5% to 99%, 5% to 90%, 5% to 80%, 5% to 70%, 5% to 60%, or 5% to 50% v/v of DMA. In some aspects, the solvent system comprises 5% to 40%, 5% to 35%, 5% to 30%, 5% to 25%, 5% to 20%, 5% to 15%, or 5% to 10% v/v of DMA.

In some aspects, the solvent system comprises about 10% to about 99%, about 10% to about 90%, about 10% to about 80%, about 10% to about 70%, about 10% to about 60%, or about 10% to about 50% v/v of DMA. In some aspects, the solvent system comprises about 10% to about 40%, about 10% to about 35%, about 10% to about 30%, about 10% to about 25%, about 10% to about 20%, or about 10% to about 15% v/v of DMA.

In some aspects, the solvent system comprises 10% to 99%, 10% to 90%, 10% to 80%, 10% to 70%, 10% to 60%, or 10% to 50% v/v of DMA. In some aspects, the solvent system comprises 10% to 40%, 10% to 35%, 10% to 30%, 10% to 25%, 10% to 20%, or 10% to 15% v/v of DMA.

In some aspects, the solvent system comprises about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, or about 30% v/v of DMA.

In some aspects, the solvent system comprises 10%, 11%, 12%, 13%, 14%, 15%, 16%, 17%, 18%, 19%, 20%, 21%, 22%, 23%, 24%, 25%, 26%, 27%, 28%, 29%, or 30% v/v of DMA.

In some aspects, the formulation has a pH of about 1 to about 8. In some aspects, the formulation has a pH of about 1 to about 2, about 1 to about 3, about 1 to about 4, about 1 to about 5, about 1 to about 6, about 1 to about 7, or about 1 to about 8.

In some aspects, the formulation has a pH of 1 to 8. In some aspects, the formulation has a pH of 1 to 2, 1 to 3, 1 to 4, 1 to 5, 1 to 6, 1 to 7, or 1 to 8.

In some aspects, the formulation has a pH of about 2.5 to about 5. In some aspects, the formulation has a pH of about 2.5 to about 3, about 2.5 to about 3.5, about 2.5 to about 4, about 2.5 to about 4.5, or about 2.5 to about 5. In some aspects, the formulation has a pH of about 2.5 to about 3.5, about 2.6 to about 3.4, about 2.7 to about 3.3, about 2.8 to about 3.2, or about 2.9 to about 3.1.

In some aspects, the formulation has a pH of 2.5 to 5. In some aspects, the formulation has a pH of 2.5 to 3, 3 to 3.5, 2.5 to 3.5, 2.5 to 4, 2.5 to 4.5, or 2.5 to 5. In some aspects, the formulation has a pH of 2.5 to 3.5, 2.6 to 3.4, 2.7 to 3.3, 2.8 to 3.2, or 2.9 to 3.1.

In some aspects, the formulation has a pH of about 2 to about 4. In some aspects, the formulation has a pH of about 2, about 2.1, about 2.2, about 2.3, about 2.4, about 2.5, about 2.6, about 2.7, about 2.8, about 2.9, about 3.0, about 3.1, about 3.2, about 3.3, about 3.4, about 3.5, about 3.6, about 3.7, about 3.8, about 3.9, or about 4.

In some aspects, the formulation has a pH of 2 to 4. In some aspects, the liquid pharmaceutical formulation has a pH of 2, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, or 4.

In some aspects, the formulation can comprise an anticoagulant. In some aspects, the formulation can comprise heparin. In some aspects, the formulation can comprise fondaparinux.

In some aspects, the formulation does not comprise an anticoagulant. In some aspects, the formulation does not comprise heparin. In some aspects, the formulation does not comprise fondaparinux. Without being bound to any particular theory, it was surprisingly found that the formulations described herein include a buffer that can also act as an anticoagulant and therefore an additional anticoagulant, such as heparin or fondaparinux, is not needed in the formulation.

In some aspects, the formulation can have a viscosity of about 0.7 cP to about 2.5 cP. In some aspects, the formulation can have a viscosity of about 0.7 cP to about 2.2 cP, about 0.7 cP to about 2 cP, about 0.7 cP to about 1.9 cP, about 0.7 cP to about 1.8 cP, about 0.7 cP to about 1.7 cP, about 0.7 cP to about 1.6 cP, about 0.7 cP to about 1.5 cP, about 0.7 cP to about 1.4 cP, about 0.7 cP to about 1.3 cP, about 0.7 cP to about 1.2 cP, about 0.7 cP to about 1.1 cP, or about 0.7 cP to about 1 cP. In some aspects, the formulation can have a viscosity of about 1 cP to about 2.5 cP, about 1 cP to about 2.2 cP, about 1 cP to about 2 cP, about 1 cP to about 1.9 cP, about 1 cP to about 1.8 cP, about 1 cP to about 1.7 cP, about 1 cP to about 1.6 cP, about 1 cP to about 1.5 cP, about 1 cP to about 1.4 cP, about 1 cP to about 1.3 cP, about 1 cP to about 1.2 cP, or about 1 cP to about 1.1 cP.

In some aspects, the formulation can have a viscosity of 0.7 cP to 2.5 cP. In some aspects, the formulation can have a viscosity of 0.7 cP to 2.2 cP, 0.7 cP to 2 cP, 0.7 cP to 1.9 cP, 0.7 cP to 1.8 cP, 0.7 cP to 1.7 cP, 0.7 cP to 1.6 cP, 0.7 cP to 1.5 cP, 0.7 cP to 1.4 cP, 0.7 cP to 1.3 cP, 0.7 cP to 1.2 cP, 0.7 cP to 1.1 cP, or 0.7 cP to 1 cP. In some aspects, the formulation can have a viscosity of 1 cP to 2.5 cP, 1 cP to 2.2 cP, 1 cP to 2 cP, 1 cP to 1.9 cP, 1 cP to 1.8 cP, 1 cP to 1.7 cP, 1 cP to 1.6 cP, 1 cP to 1.5 cP, 1 cP to 1.4 cP, 1 cP to 1.3 cP, 1 cP to 1.2 cP, or 1 cP to 1.1 cP.

In certain aspects, the formulation can have a viscosity of about 0.7 cP to about 1.6 cP, about 0.8 cP to about 1.5 cP, about 0.9 cP to about 1.4 cP, about 1 cP to about 1.3 cP, or about 1.1 cP to about 1.2 cP.

In certain aspects, the liquid pharmaceutical formulation can have a viscosity of 0.7 cP to 1.6 cP, 0.8 cP to 1.5 cP, 0.9 cP to 1.4 cP, 1 cP to 1.3 cP, or 1.1 cP to 1.2 cP.

In some aspects, the formulation can have a viscosity of about 0.7 cP, about 0.8 cP, about 0.9 cP, about 1 cP, about 1.1 cP, about 1.2 cP, about 1.3 cP, about 1.4 cP, about 1.5 cP, or about 1.6 cP.

In some aspects, the formulation can have a viscosity of 0.7 cP, 0.8 cP, 0.9 cP, 1 cP, 1.1 cP, 1.2 cP, 1.3 cP, 1.4 cP, 1.5 cP, or 1.6 cP.

In some aspects, the formulation comprises about 50 mg/ml to about 150 mg/ml of trifluridine, about 5 mM to about 250 mM of buffer, and a solvent system comprising water and a co-solvent, wherein the formulation has a pH of about 1 to 8. In some aspects, the formulation can have a viscosity of about 0.7 cP to about 2.5 cP. In some aspects, the formulation has a viscosity of about 1 cP to about 1.5 cP. In certain aspects, the formulation has a viscosity of about 1.1 cP to about 1.2 cP.

In some aspects, the formulation comprises 50 mg/ml to 150 mg/ml of trifluridine, 5 mM to 250 mM of buffer, and a solvent system comprising water and a co-solvent, wherein the formulation has a pH of about 1 to 8. In some aspects, the formulation can have a viscosity of 0.7 cP to 2.5 cP. In some aspects, the formulation has a viscosity of 1 cP to 1.5 cP. In certain aspects, the formulation has a viscosity of 1.1 cP to 1.2 cP.

In some aspects, the formulation consists essentially of about 50 mg/ml to about 150 mg/ml of trifluridine, about 5 mM to about 250 mM of buffer, and a solvent system comprising water and a co-solvent, wherein the formulation has a pH of about 1 to 8. In some aspects, the formulation can have a viscosity of about 0.7 cP to about 2.5 cP. In some aspects, the formulation has a viscosity of about 1 cP to about 1.5 cP. In certain aspects, the formulation has a viscosity of about 1.1 cP to about 1.2 cP.

In some aspects, the formulation consists essentially of 50 mg/ml to 150 mg/ml of trifluridine, 5 mM to 250 mM of buffer, and a solvent system comprising water and a co-solvent, wherein the formulation has a pH of about 1 to 8. In some aspects, the formulation can have a viscosity of 0.7 cP to 2.5 cP. In some aspects, the formulation has a viscosity of 1 cP to 1.5 cP. In certain aspects, the formulation has a viscosity of 1.1 cP to 1.2 cP.

In some aspects, the formulation consists of about 50 mg/ml to about 150 mg/ml of trifluridine, about 5 mM to about 250 mM of buffer, and a solvent system comprising water and a co-solvent, wherein the formulation has a pH of about 1 to 8. In some aspects, the formulation can have a viscosity of about 0.7 cP to about 2.5 cP. In some aspects, the formulation has a viscosity of about 1 cP to about 1.5 cP. In certain aspects, the formulation has a viscosity of about 1.1 cP to about 1.2 cP.

In some aspects, the formulation consists of 50 mg/ml to 150 mg/ml of trifluridine, 5 mM to 250 mM of buffer, and a solvent system comprising water and a co-solvent, wherein the formulation has a pH of about 1 to 8. In some aspects, the formulation can have a viscosity of 0.7 cP to 2.5 cP. In some aspects, the formulation has a viscosity of 1 cP to 1.5 cP. In certain aspects, the formulation has a viscosity of 1.1 cP to 1.2 cP.

In some aspects, the formulation comprises about 50 mg/ml to about 100 mg/ml of trifluridine, about 25 mM to about 100 mM of citrate buffer, a solvent system comprising water and about 10% to about 30% v/v DMA, wherein the formulation has a pH of about 2 to 5. In some aspects, the formulation can have a viscosity of about 0.7 cP to about 2.5 cP. In some aspects, the formulation has a viscosity of about 1 cP to about 1.5 cP. In certain aspects, the formulation has a viscosity of about 1.1 cP to about 1.2 cP.

In some aspects, the formulation comprises 50 mg/ml to 100 mg/ml of trifluridine, 25 mM to 100 mM of citrate buffer, a solvent system comprising water and 10% to 30% v/v DMA, wherein the formulation has a pH of 2 to 5. In some aspects, the formulation can have a viscosity of 0.7 cP to 2.5 cP. In some aspects, the formulation has a viscosity of 1 cP to 1.5 cP. In certain aspects, the formulation has a viscosity of 1.1 cP to 1.2 cP.

In some aspects, the formulation consists essentially of about 50 mg/ml to about 100 mg/ml of trifluridine, about 25 mM to about 100 mM of citrate buffer, a solvent system comprising water and about 10% to 30% v/v DMA, wherein the formulation has a pH of about 2 to 5. In some aspects, the formulation has a pH of about 3. In some aspects, the formulation can have a viscosity of about 0.7 cP to about 2.5 cP. In some aspects, the formulation has a viscosity of about 1 cP to about 1.5 cP. In certain aspects, the formulation has a viscosity of about 1.1 cP to about 1.2 cP.

In some aspects, the formulation consists essentially of 50 mg/ml to 100 mg/ml of trifluridine, 25 mM to 100 mM of citrate buffer, a solvent system comprising water and 10% to 30% v/v DMA, wherein the formulation has a pH of 2 to 5. In some aspects, the formulation has a pH of 3. In some aspects, the formulation can have a viscosity of 0.7 cP to 2.5 cP. In some aspects, the formulation has a viscosity of 1 cP to 1.5 cP. In certain aspects, the formulation has a viscosity of 1.1 cP to 1.2 cP.

In some aspects, the formulation consists of about 50 mg/ml to about 100 mg/ml of trifluridine, about 25 mM to about 100 mM of citrate buffer, a solvent system comprising water and about 10% to about 30% v/v DMA, wherein the formulation has a pH of about 2 to 5. In some aspects, the formulation has a pH of about 3. In some aspects, the formulation can have a viscosity of about 0.7 cP to about 2.5 cP. In some aspects, the formulation has a viscosity of about 1 cP to about 1.5 cP. In certain aspects, the formulation has a viscosity of about 1.1 cP to about 1.2 cP.

In some aspects, the formulation consists of 50 mg/ml to 100 mg/ml of trifluridine, 25 mM to 100 mM of citrate buffer, a solvent system comprising water and 10% to 30% v/v DMA, wherein the formulation has a pH of 2 to 5. In some aspects, the formulation has a pH of about 3. In some aspects, the formulation can have a viscosity of 0.7 cP to 2.5 cP. In some aspects, the formulation has a viscosity of 1 cP to 1.5 cP. In certain aspects, the formulation has a viscosity of 1.1 cP to 1.2 cP.

In some aspects, the formulation comprises about 50 mg/ml to about 100 mg/ml of trifluridine, about 25 mM to about 100 mM of citrate buffer, a solvent system comprising water and about 10% to about 20% v/v DMA, wherein the formulation has a pH of about 2 to 5. In some aspects, the formulation can have a viscosity of about 0.7 cP to about 2.5 cP. In some aspects, the formulation has a viscosity of about 1 cP to about 1.5 cP. In certain aspects, the formulation has a viscosity of about 1.1 cP to about 1.2 cP.

In some aspects, the formulation comprises 50 mg/ml to 100 mg/ml of trifluridine, 25 mM to 100 mM of citrate buffer, a solvent system comprising water and 10% to 20% v/v DMA, wherein the formulation has a pH of 2 to 5. In some aspects, the formulation can have a viscosity of 0.7 cP to 2.5 cP. In some aspects, the formulation has a viscosity of 1 cP to 1.5 cP. In certain aspects, the formulation has a viscosity of 1.1 cP to 1.2 cP.

In some aspects, the formulation consists essentially of about 50 mg/ml to about 100 mg/ml of trifluridine, about 25 mM to about 100 mM of citrate buffer, a solvent system comprising water and about 10% to about 20% v/v DMA, wherein the formulation has a pH of about 2 to 5. In some aspects, the formulation has a pH of about 3. In some aspects, the formulation can have a viscosity of about 0.7 cP to about 2.5 cP. In some aspects, the formulation has a viscosity of about 1 cP to about 1.5 cP. In certain aspects, the formulation has a viscosity of about 1.1 cP to about 1.2 cP.

In some aspects, the formulation consists essentially of 50 mg/ml to 100 mg/ml of trifluridine, 25 mM to 100 mM of citrate buffer, a solvent system comprising water and 10% to 20% v/v DMA, wherein the formulation has a pH of 2 to 5. In some aspects, the formulation has a pH of 3. In some aspects, the formulation can have a viscosity of 0.7 cP to 2.5 cP. In some aspects, the formulation has a viscosity of 1 cP to 1.5 cP. In certain aspects, the formulation has a viscosity of 1.1 cP to 1.2 cP.

In some aspects, the formulation consists of about 50 mg/ml to about 100 mg/ml of trifluridine, about 25 mM to about 100 mM of citrate buffer, a solvent system comprising water and about 10% to about 20% v/v DMA, wherein the formulation has a pH of about 2 to 5. In some aspects, the formulation has a pH of about 3. In some aspects, the formulation can have a viscosity of about 0.7 cP to about 2.5 cP. In some aspects, the formulation has a viscosity of about 1 cP to about 1.5 cP. In certain aspects, the formulation has a viscosity of about 1.1 cP to about 1.2 cP.

In some aspects, the formulation consists of 50 mg/ml to 100 mg/ml of trifluridine, 25 mM to 100 mM of citrate buffer, a solvent system comprising water and 10% to 20% v/v DMA, wherein the formulation has a pH of 2 to 5. In some aspects, the formulation has a pH of about 3. In some aspects, the formulation can have a viscosity of 0.7 cP to 2.5 cP. In some aspects, the formulation has a viscosity of 1 cP to 1.5 cP. In certain aspects, the formulation has a viscosity of 1.1 cP to 1.2 cP.

In some aspects, the formulation comprises about 70 mg/ml of trifluridine, about 75 mM citrate buffer, a solvent system comprising water and about 14% v/v DMA, wherein the formulation has a pH of about 3. In certain aspects, the formulation has a viscosity of about 1.1 cP to about 1.2 cP.

In some aspects, the formulation comprises 70 mg/ml of trifluridine, about 75 mM citrate buffer, a solvent system comprising water and about 14% v/v DMA, wherein the formulation has a pH of about 3. In certain aspects, the formulation has a viscosity of 1.1 cP to 1.2 cP.

In some aspects, the formulation consists essentially of about 70 mg/ml of trifluridine, about 75 mM citrate buffer, a solvent system comprising water and about 14% v/v DMA, wherein the formulation has a pH of about 3. In certain aspects, the formulation has a viscosity of 1.1 cP to 1.2 cP.

In some aspects, the formulation consists about 70 mg/ml of trifluridine, about 75 mM citrate buffer, a solvent system comprising water and about 14% v/v DMA, wherein the formulation has a pH of about 3 and a viscosity of about 1.1 cP to about 1.2 cP.

### Additional Methods of Treatment

As previously indicated, the present disclosure also discloses methods of treatment of liver cancer by administration of the formulation disclosed herein by HAI. Without being bound to any particular theory, it is surprisingly found that the formulation disclosed herein increase the amount of trifluridine that can be administered to a subject in need thereof on a daily basis to increase potential for efficacy in the treatment of cancer.

In some aspects, the subject can be a subject with primary liver cancer. In some aspects, the subject can be a subject with secondary liver cancer. In some aspects, the subject can be a subject with colon cancer that has spread to the liver. In some aspects, the subject can be a subject with cancer that started in the bile duct within the liver (intrahepatic cholangiocarcinoma). In some aspects, the subject can be a subject with hepatocellular carcinoma. In some aspects, the subject can be a subject with resectable cancer. In some aspects, the subject can be a subject with unresectable cancer.

In some aspects, the disclosure provides a method of treating liver cancer in a subject in need thereof, the method comprising administering to the subject a therapeutically effective amount of the formulation as described herein by hepatic arterial infusion. In some aspects, the therapeutically effective amount of the formulation is administered by hepatic arterial infusion via a pump. Hepatic artery infusion pumps known by and available to one of ordinary skill in the art is used to administer trifluridine, including, but not limited to, implantable pumps, external pumps, continuous flow pumps, peristalsis pumps, and programmable pumps. Examples of HAI pumps may include, but are not limited to, INTERA® 3000 HAI Pump. A peristalsis pump can be a programmable pump, a battery operated pump, or any other pump that is not a mechanical constant flow pump.

In some aspects, the therapeutically effective amount of the formulation comprises about 0.01 mg/kg/day to about 5 mg/kg/day of trifluridine. In some aspects, the therapeutically effective amount of the formulation comprises about 0.01 mg/kg/day to about 4 mg/kg/day, about 0.01 mg/kg/day to about 3 mg/kg/day, about 0.01 mg/kg/day to about 2 mg/kg/day, about 0.01 mg/kg/day to about 1 mg/kg/day, about 0.01 mg/kg/day to about 0.9 mg/kg/day, about 0.01 mg/kg/day to about 0.8 mg/kg/day, about 0.01 mg/kg/day to about 0.7 mg/kg/day, about 0.01 mg/kg/day to about 0.6 mg/kg/day of trifluridine, about 0.01 mg/kg/day to about 0.5 mg/kg/day, about 0.01 mg/kg/day to about 0.4 mg/kg/day, about 0.01 mg/kg/day to about 0.3 mg/kg/day, about 0.01 mg/kg/day to about 0.2 mg/kg/day, about 0.01 mg/kg/day to about 0.1 mg/kg/day of trifluridine.

In some aspects, the therapeutically effective amount of the formulation comprises 0.01 mg/kg/day to 5 mg/kg/day of trifluridine. In some aspects, the therapeutically effective amount of the formulation comprises 0.01 mg/kg/day to 4 mg/kg/day, 0.01 mg/kg/day to 3 mg/kg/day, 0.01 mg/kg/day to 2 mg/kg/day, 0.01 mg/kg/day to 1 mg/kg/day, 0.01 mg/kg/day to 0.9 mg/kg/day, 0.01 mg/kg/day to 0.8 mg/kg/day, 0.01 mg/kg/day to 0.7 mg/kg/day, 0.01 mg/kg/day to 0.6 mg/kg/day of trifluridine, 0.01 mg/kg/day to 0.5 mg/kg/day, 0.01 mg/kg/day to 0.4 mg/kg/day, 0.01 mg/kg/day to 0.3 mg/kg/day, 0.01 mg/kg/day to 0.2 mg/kg/day, 0.01 mg/kg/day to 0.1 mg/kg/day of trifluridine.

In some aspects, the therapeutically effective amount of the formulation comprises about 0.1 mg/kg/day to about 5 mg/kg/day of trifluridine. In some aspects, the therapeutically effective amount of the formulation comprises about 0.1 mg/kg/day to about 4 mg/kg/day, about 0.1 mg/kg/day to about 3 mg/kg/day, about 0.1 mg/kg/day to about 2 mg/kg/day, about 0.1 mg/kg/day to about 1 mg/kg/day, about 0.1 mg/kg/day to about 0.9 mg/kg/day, about 0.1 mg/kg/day to about 0.8 mg/kg/day, about 0.1 mg/kg/day to about 0.7 mg/kg/day, about 0.1 mg/kg/day to about 0.6 mg/kg/day, about 0.1 mg/kg/day to about 0.5 mg/kg/day, about 0.1 mg/kg/day to about 0.4 mg/kg/day, about 0.1 mg/kg/day to about 0.3 mg/kg/day, about 0.1 mg/kg/day to about 0.2 mg/kg/day of trifluridine.

In some aspects, the therapeutically effective amount of the formulation comprises 0.1 mg/kg/day to 5 mg/kg/day of trifluridine. In some aspects, the therapeutically effective amount of the formulation comprises 0.1 mg/kg/day to 4 mg/kg/day, 0.1 mg/kg/day to 3 mg/kg/day, 0.1 mg/kg/day to 2 mg/kg/day, 0.1 mg/kg/day to 1 mg/kg/day, 0.1 mg/kg/day to 0.9 mg/kg/day, 0.1 mg/kg/day to 0.8 mg/kg/day, 0.1 mg/kg/day to 0.7 mg/kg/day, 0.1 mg/kg/day to 0.6 mg/kg/day, 0.1 mg/kg/day to 0.5 mg/kg/day, 0.1 mg/kg/day to 0.4 mg/kg/day, 0.1 mg/kg/day to 0.3 mg/kg/day, 0.1 mg/kg/day to 0.2 mg/kg/day of trifluridine.

In some aspects, the therapeutically effective amount of the formulation comprises about 0.5 mg/kg/day to about 5 mg/kg/day of trifluridine. In some aspects, the therapeutically effective amount of the formulation comprises about 0.5 mg/kg/day to about 0.6 mg/kg/day, about 0.5 mg/kg/day to about 0.7 mg/kg/day, about 0.5 mg/kg/day to about 0.8 mg/kg/day, about 0.5 mg/kg/day to about 0.9 mg/kg/day, about 0.5 mg/kg/day to about 1 mg/kg/day, about 0.5 mg/kg/day to about 1.1 mg/kg/day, 0.5 mg/kg/day to about 1.2 mg/kg/day, 0.5 mg/kg/day to about 1.3 mg/kg/day, 0.5 mg/kg/day to about 1.4 mg/kg/day, about 0.5 mg/kg/day to about 1.5 mg/kg/day of trifluridine, about 0.5 mg/kg/day to about 2 mg/kg/day of trifluridine, about 0.5 mg/kg/day to about 2.5 mg/kg/day of trifluridine, about 0.5 mg/kg/day to about 3 mg/kg/day of trifluridine, about 0.5 mg/kg/day to about 3.5 mg/kg/day of trifluridine, about 0.5 mg/kg/day to about 4 mg/kg/day of trifluridine, about 0.5 mg/kg/day to about 4.5 mg/kg/day of trifluridine, or about 0.5 mg/kg/day to about 5 mg/kg/day of trifluridine.

In some aspects, the therapeutically effective amount of the formulation comprises 0.5 mg/kg/day to 5 mg/kg/day of trifluridine. In some aspects, the therapeutically effective amount of the formulation comprises 0.5 mg/kg/day to 0.6 mg/kg/day, 0.5 mg/kg/day to 0.7 mg/kg/day, 0.5 mg/kg/day to 0.8 mg/kg/day, 0.5 mg/kg/day to 0.9 mg/kg/day, 0.5 mg/kg/day to 1 mg/kg/day, 0.5 mg/kg/day to 1.1 mg/kg/day, 0.5 mg/kg/day to 1.2 mg/kg/day, 0.5 mg/kg/day to 1.3 mg/kg/day, 0.5 mg/kg/day to 1.4 mg/kg/day, 0.5 mg/kg/day to 1.5 mg/kg/day of trifluridine, 0.5 mg/kg/day to 2 mg/kg/day of trifluridine, 0.5 mg/kg/day to 2.5 mg/kg/day of trifluridine, 0.5 mg/kg/day to 3 mg/kg/day of trifluridine, 0.5 mg/kg/day to 3.5 mg/kg/day of trifluridine, 0.5 mg/kg/day to 4 mg/kg/day of trifluridine, 0.5 mg/kg/day to 4.5 mg/kg/day of trifluridine, or 0.5 mg/kg/day to 5 mg/kg/day of trifluridine.

In some aspects, the therapeutically effective amount of the formulation comprises about 0.5 mg/kg/day to about 1.5 mg/kg/day of trifluridine. In some aspects, the therapeutically effective amount of the formulation comprises about 0.6 mg/kg/day, about 0.7 mg/kg/day, about 0.8 mg/kg/day, about 0.9 mg/kg/day, about 1 mg/kg/day, about 1.1 mg/kg/day, about 1.2 mg/kg/day, about 1.3 mg/kg/day, about 1.4 mg/kg/day, or about 1.5 mg/kg/day of trifluridine.

In some aspects, the therapeutically effective amount of the formulation comprises 0.5 mg/kg/day to 1.5 mg/kg/day of trifluridine. In some aspects, the therapeutically effective amount of the formulation comprises 0.6 mg/kg/day, 0.7 mg/kg/day, 0.8 mg/kg/day, 0.9 mg/kg/day, 1 mg/kg/day, 1.1 mg/kg/day, 1.2 mg/kg/day, 1.3 mg/kg/day, 1.4 mg/kg/day, or 1.5 mg/kg/day of trifluridine.

It is to be appreciated that the Detailed Description section, and not the Summary and Abstract sections, is intended to be used to interpret the claims. The Summary and Abstract sections may set forth one or more but not all exemplary aspects of the present disclosure as contemplated by the inventor(s), and thus, are not intended to limit the present disclosure and the appended claims in any way.

### EXAMPLES

### Example 1: Hepatic Arterial Infusion of Trifluridine to Assess Liver Extraction

A 4-hour continuous hepatic arterial infusion of trifluridine was performed in three monkeys to assess whether trifluridine is highly extracted by the liver. A dose of 0.62 mg/kg infused continuously over the course of 4 hours (0.155 mg/kg/hr) was chosen to be equivalent to the human trifluridine dose of 0.05 mg/kg/hr, based on standard body surface area (BSA) conversion ration between humans and monkeys of 3.1.

Analysis of the hepatic extraction ratio (HER) was performed similarly to the method described by Ensminger et al. (1978), “A Clinical-Pharmacological Evaluation of Hepatic Arterial Infusions of 5-Fluoro-2′-deoxyuridine and 5-Fluorouracil,” Cancer Res., 38(11 part 1): 3784-3792 by measuring total hepatic blood flow and sampling the hepatic vein for the concentration of trifluridine at six time points (5 minutes, 15 minutes, 30 minutes, 60 minutes, 120 minutes, and 240 minutes). The efflux of the drug versus the known influx of the drug infusion was measured.

Two of the three monkeys exhibited significant extraction of trifluridine by the liver and the third monkey exhibited medium extraction (Table 1).

Trifluridine was shown to be highly extracted by the liver in monkeys. Human intrinsic clearance (CLint) is higher than monkey CLint, so HER is expected to be similar or higher than those in monkeys.

TABLE 1
Hepatic extraction ratio (HER) of trifluridine in three monkeys.

| | HER (%) - | HER (%) - | HER (%) - | HER (%) - | HER (%) - | HER (%) - |
|---|---|---|---|---|---|---|
| Animal # | 5 min | 15 min | 30 min | 60 min | 120 min | 240 min |
| 1 | N/A | N/A | 88.8 | 79.2 | 57.4 | 98.2 |
| 2 | 98.9 | 85.7 | 81.0 | 87.4 | 95.6 | 100 |
| 3 | N/A | 68.6 | 28.5 | 28.7 | 13.1 | 42.2 |

### Example 2: In Vitro Cell Assay Pharmacology to Assess Trifluridine Effectiveness

To quantify the enhanced effectiveness (measured as reduction in IC₅₀) of trifluridine in longer exposure durations, an in vitro cell cytotoxicity assay using five cell lines was performed. The cell lines included: DLD-1 (colorectal), HCT-116 (colorectal), WiDr (colorectal), Li-7 (hepatocellular carcinoma, and KKU-055 (cholangiocarcinoma). Cells were harvested from culture flasks and seeded in the optimal pre-determined cell density to 96-well culture plates in the optimal culture medium, supplemented with 10% fetal bovine serum (FBS) and 1% Penicillin/Streptomycin, at 90 μl/well. Culture plates were left overnight in a 37° C./5% CO₂ incubator overnight to allow cells to settle to the plate and adhere. The following day, trifluridine (300,000 to 45.73 ng/ml final concentration, 3× concentration reduction between treatments) or DMSO (serving as vehicle control) were dispensed into the appropriate wells at a volume of 10 μl/well for a final volume of 100 μl/well. Culture plates replicates were prepared in sufficient quantities to read plates at the following time points after introduction of trifluridine: 1, 3, 7, and 14 days. At the relevant time points, cell cytotoxicity was measured using the Promega CellTiter-Glo (CTG) method which includes a substrate that generates light based on the available cellular ATP quantities. The CTG reagent was thawed and allowed to reach room temperature, then 100 μl/well of the reagent was added to each well, and left at room temperature for 30 minutes, following which the plates' luminescence was read by a luminometer.

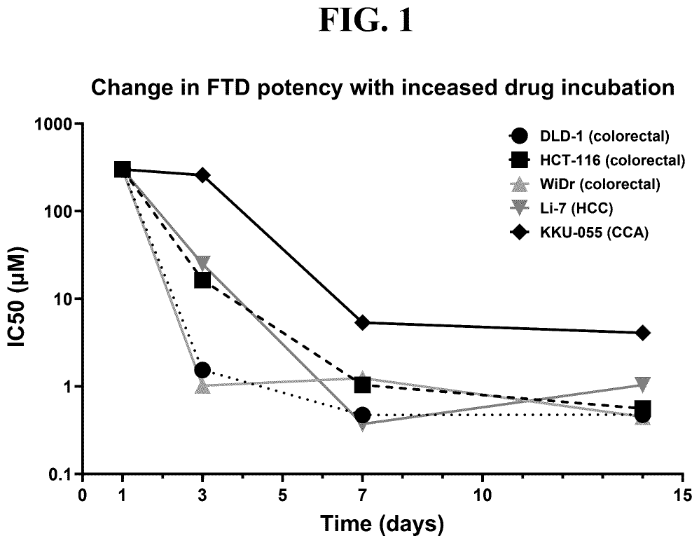

The IC₅₀ of trifluridine was measured on days 1, 3, 7, and 14. Results are shown in FIG. 1. There was a dramatic reduction in the sensitivity of the cells between days 3 and 7, up to a 10-fold decrease of IC₅₀ in the Li-7 and KKU-055 cell lines. Sensitivity of tumors to trifluridine increased with prolonged exposures. Based on these results, trifluridine is an optimal fit for a continuous infusion drug.

### Example 3: 28-Day Continuous Hepatic Arterial Infusion of Trifluridine to Assess Toxicity

Male SD-1 rats were continuously dosed via the hepatic artery for 28 days. Four groups were tested: Group 1: vehicle, n=6; Group 2: trifluridine at 0.5 mg/kg/day, n=6; Group 3: trifluridine at 1.5 mg/kg/day, n=6; and Group 4: 4.5 mg/kg/day, n=6.

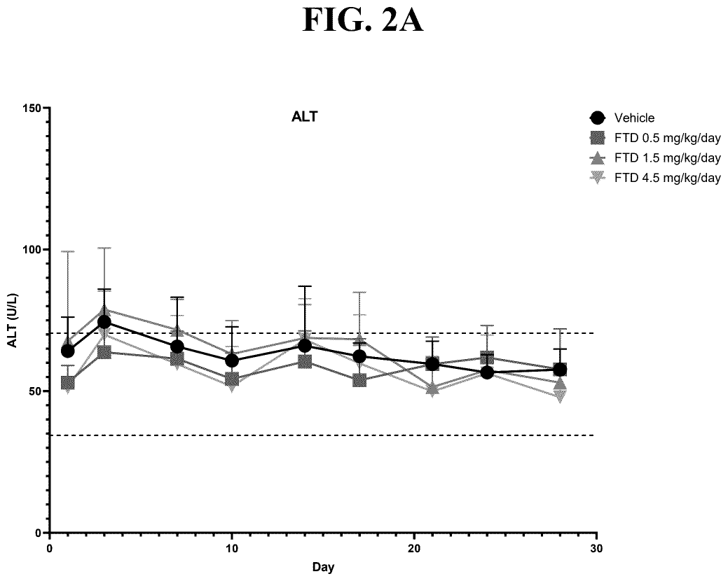

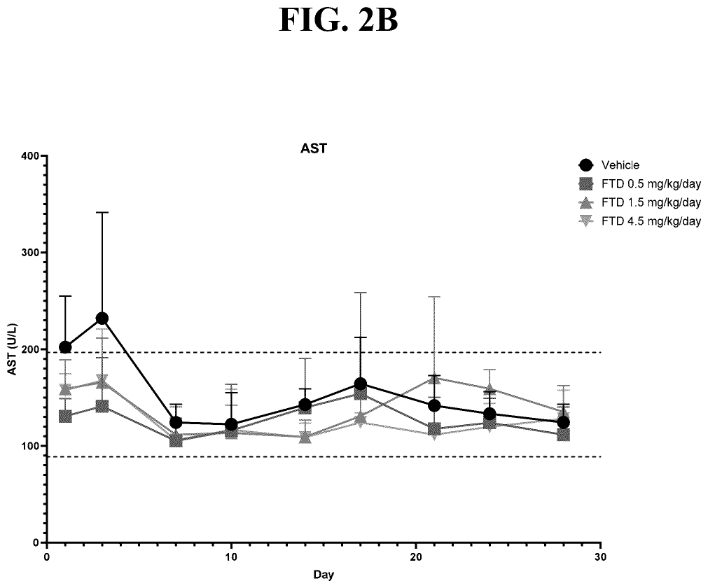

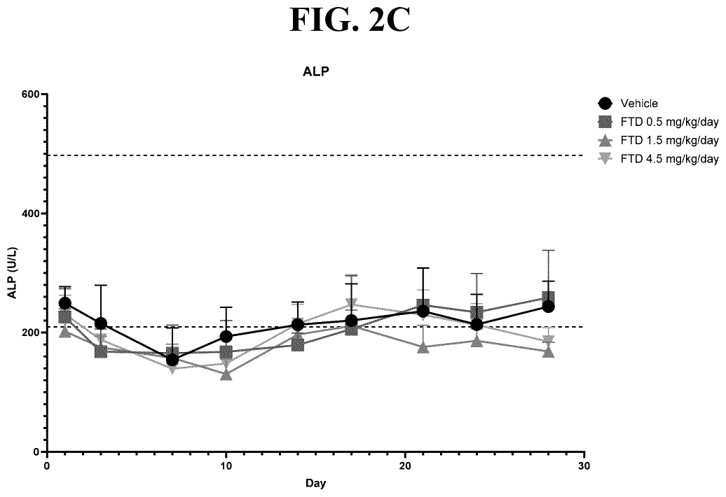

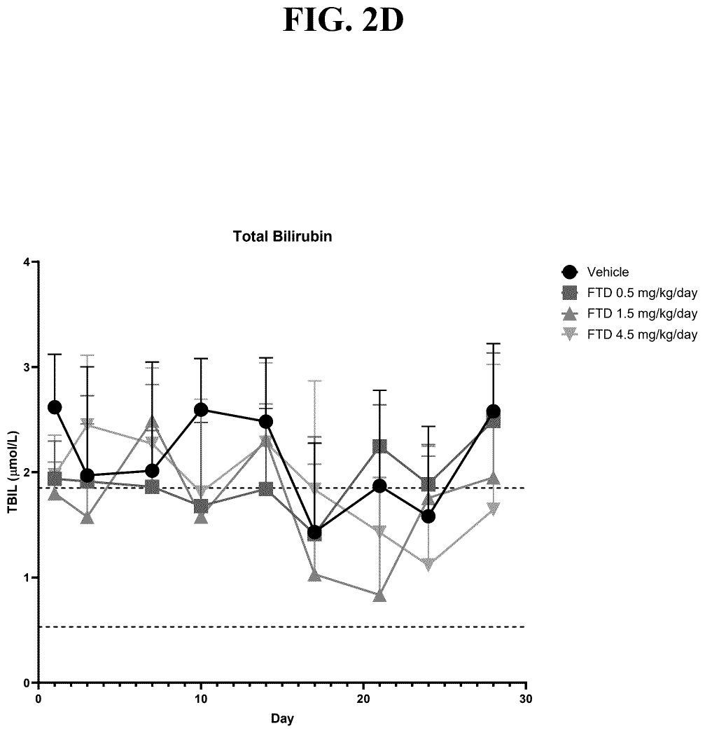

An Alzet mini-osmotic pump (mode 2ML4) was used and pumped continuously for 28 days at a rate of 2.5 μl/hr the drug solution or the vehicle as control. The alanine aminotransferase (ALT), aspartate aminotransferase (AST), alkaline phosphatase (ALP), and total bilirubin levels of the rats were measured over time. No liver/biliary toxicity was observed with 28 days of continuous infusion of trifluridine at doses up to 4.5 mg/kg/day. Results are shown in FIGS. 2A-2D.

Hepatic arterial infusion of trifluridine at doses of up to a human equivalent dose of 0.75 mg/kg/day did not generate liver/bile toxicity.

### Example 4: 28-Day Continuous Hepatic Arterial Infusion of Trifluridine to Assess Toxicity

Male SD-1 rats were continuously dosed via the hepatic artery for 28 days. Four groups were tested: Group 1: saline, n=6, Group 2: vehicle, n=6; Group 3: trifluridine at 5 mg/kg/day, n=6; and Group 4: trifluridine at 15 mg/kg/day, n=6.

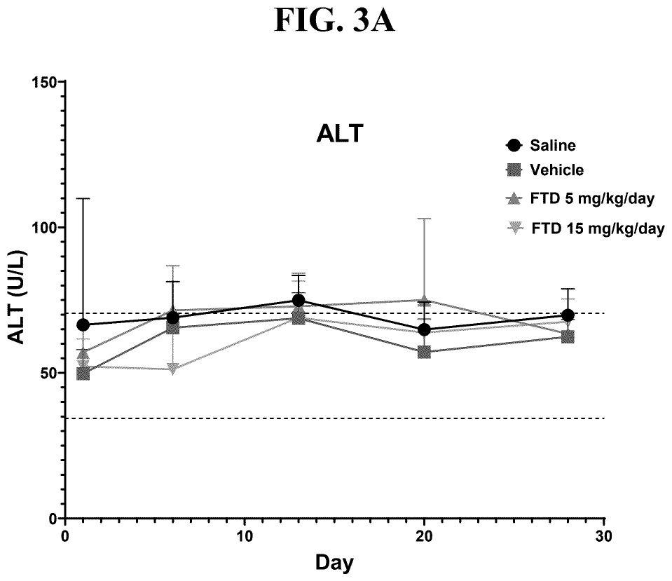

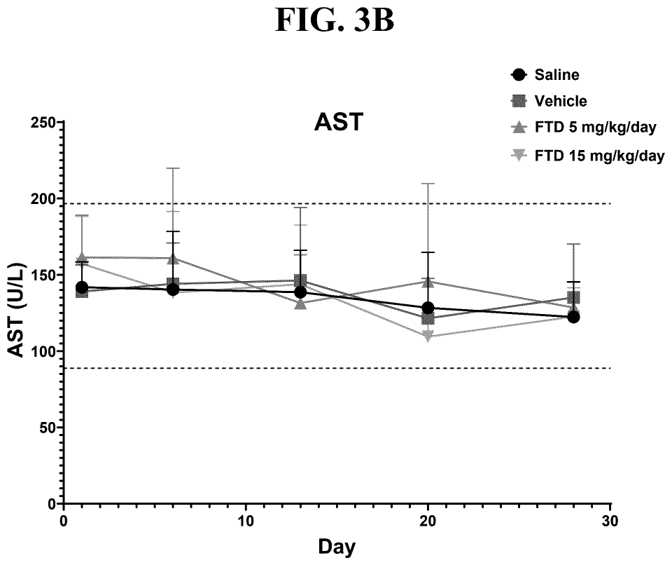

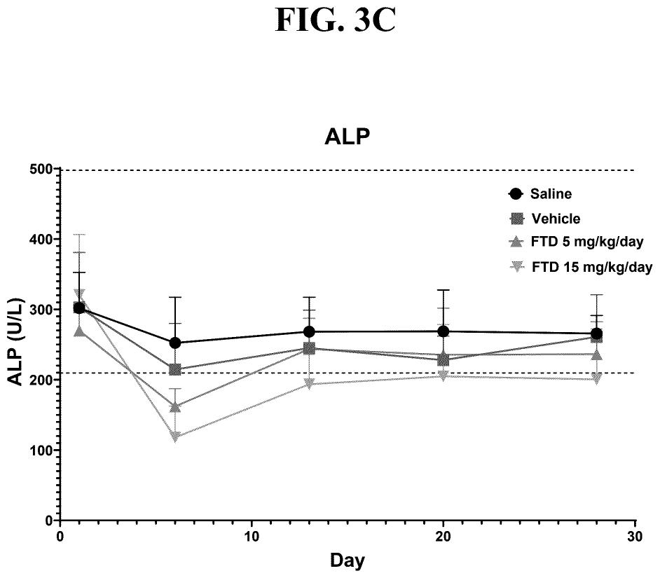

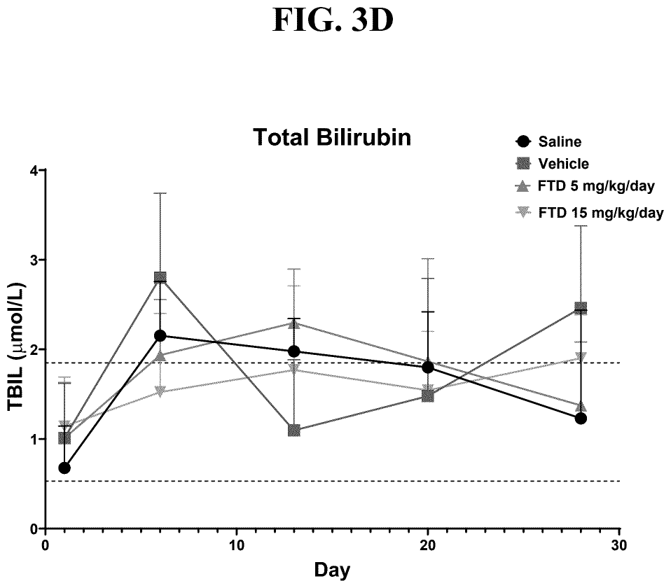

An Alzet mini-osmotic pump (mode 2ML4) was used and pumped continuously for 28 days at a rate of 2.5 μl/hr for the drug solution, the saline or the vehicle as controls. The alanine aminotransferase (ALT), aspartate aminotransferase (AST), alkaline phosphatase (ALP), and total bilirubin levels of the rats were measured over time. No liver/biliary toxicity was observed with 28 days of continuous infusion of trifluridine at doses up to 15 mg/kg/day based on liver function tests and liver histopathology. Results of the liver function tests are shown in FIGS. 3A-3D.

Hepatic arterial infusion of trifluridine at doses of up to a human equivalent dose of 2.42 mg/kg/day did not generate liver/bile toxicity.

### Example 5: Treating Liver Cancer with Trifluridine Continuously Administered for 7 Days

A subject having liver cancer is administered a therapeutically effective amount of trifluridine by hepatic arterial infusion via an internal pump continuously for 7 days. Trifluridine by hepatic arterial infusion inhibits cellular proliferation, inhibits tumor growth, and treats liver cancer. The subject's alanine aminotransferase, aspartate aminotransferase, alkaline phosphatase, and bilirubin levels do not rise more than about 20% above the subject's alanine aminotransferase, aspartate aminotransferase, alkaline phosphatase, and bilirubin levels prior to administration of the therapeutically effective amount of trifluridine.

### Example 6: Treating Liver Cancer with Trifluridine Continuously Administered for 14 Days

A subject having liver cancer is administered a therapeutically effective amount of trifluridine by hepatic arterial infusion via an internal pump continuously for 14 days. Trifluridine by hepatic arterial infusion inhibits cellular proliferation, inhibits tumor growth, and treats liver cancer. The subject's alanine aminotransferase, aspartate aminotransferase, alkaline phosphatase, and bilirubin levels do not rise more than about 20% above the subject's alanine aminotransferase, aspartate aminotransferase, alkaline phosphatase, and bilirubin levels prior to administration of the therapeutically effective amount of trifluridine.

### Example 7: Treating Liver Cancer with Trifluridine Continuously Administered for 28 Days

A subject having liver cancer is administered a therapeutically effective amount of trifluridine by hepatic arterial infusion via an internal pump continuously for 28 days. Trifluridine by hepatic arterial infusion inhibits cellular proliferation, inhibits tumor growth, and treats liver cancer. The subject's alanine aminotransferase, aspartate aminotransferase, alkaline phosphatase, and bilirubin levels do not rise more than about 20% above the subject's alanine aminotransferase, aspartate aminotransferase, alkaline phosphatase, and bilirubin levels prior to administration of the therapeutically effective amount of trifluridine.

### Example 8

The effect of various co-solvents on trifluridine solubility was evaluated based on the following procedure.

An aqueous phase was prepared, including 50 mM citric acid, 1,000 IU/mL heparin sodium, at pH 3.0 (adjusted with HCl and NaOH as needed). The vehicles for solubility investigation were also prepared with 25 v/v % of each of the following solvents along with 75% aqueous phase: ethanol, propylene glycol, dimethyl sulfoxide, dimethylacetamide, N-methyl-2-pyrrolidone, PEG 400, and water (the control was still mixed with 75% aqueous phase).

The pH of each cosolvent system and water was tested and recorded. To prepare and analyze the samples, ˜50 mg trifluridine was weighed into 14 separate Whatman Mini-UniPrep Syringeless Filters. 400 μL of each solvent system was added, in duplicate, to the trifluridine-containing filter systems (˜100 mg/mL). Each sample was vortexed for at least 2 minutes. The samples were placed on the shaker at room temperature for ˜24 hours at 800 rpm. The samples were removed from the shaker and allowed to set at room temperature for 1-2 hours. The laboratory temperature and appearance were recorded. Samples were placed in the centrifuge at 25° C. for 10 minutes at 4,000 rpm. The samples were allowed to set at room temperature for 30-60 minutes and laboratory temperature was recorded. Each vial was compressed to filter the contents through the filtration system. The filtered concentration was about 40 mg/ml. An aliquot of the filtrate was removed for dilution with 25% acetonitrile, 75% water, and 0.1% TFA. The sample was analyzed by HPLC against standards of known concentration. The pH of each filtrate was tested and recorded.

Table 2 shows the trifluridine solubility results of the tested formulations. The introduction of DMA and NMP increased trifluridine solubility to over 100 mg/ml.

TABLE 2
Pharmaceutical Formulation Co-Solvent Analysis

| | | | | | | 25 C.- | After 25 C.-800 rpm-24 h | | | | |
|---|---|---|---|---|---|---|---|---|---|---|---|
| | | | | | | 800 rpm- | Standstill | Standstill 2 h-25 C. | | | |
| | Target | Aqueous | | T0 | | 24 h | 1 h-25 C. | | | Conc. | Purity |
| No. | API | phase | Co-solvent | Appearance | pH | Appearance | Appearance | Appearance | pH | (mg/mL) | (%) |
| PT 1 | 100 mg/ml | 50 mM | 25% Ethanol(EtOH) | Suspension | 3.66 | Suspension | Suspension | Suspension | 3.59 | 56.03 | 100.00 |
| PT 2 | | citric | 25% Propylene | Suspension | 3.52 | Suspension | Suspension | Suspension | 3.49 | 46.34 | 100.00 |
| | | acid, | glycol(PG) | | | | | | | | |
| PT 3 | | 1000 | 25% Dimethyl | Suspension | 3.89 | Suspension | Suspension | Suspension | 3.92 | 62.52 | 100.00 |
| | | IU/mL | sulfoxide(DMSO) | | | | | | | | |
| PT 4 | | heparin | 25% | Clear | 3.82 | Clear | Clear | Clear | 3.74 | 107.03 | 100.00 |
| | | sodium, | Dimethyl- | solution | | solution | solution | solution | | | |
| | | pH | acetamide(DMA) | with small | | with | with | with | | | |
| | | 3.0(75%) | | solid | | particles | particles | particles | | | |
| PT 5 | | | 25% N-methyl-2- | Clear | 3.67 | Clear | Clear | Clear | 3.83 | 114.08 | 100.00 |
| | | | pyrrolidone(NMP) | solution | | solution | solution | solution | | | |
| | | | | with small | | with | with | with | | | |
| | | | | solid | | particles | particles | particles | | | |
| PT 6 | | | 25% PEG 400 | Suspension | 3.83 | Suspension | Suspension | Suspension | 3.80 | 48.54 | 100.00 |
| PT 7 | | | 25% Water | Suspension | 3.32 | Suspension | Suspension | Suspension | 3.32 | 45.50 | 100.00 |

### Example 9

Based on the cosolvent solubility screening described in Table 2, DMA was selected as the cosolvent, and a further investigation of FTD solubility as a function of DMA concentration was conducted in the presence of heparin. Based on FTD solubility as a function of DMA concentration, a series of formulations with heparin were prepared for viscosity determination to assess the effect of DMA concentration on an achievable FTD daily dose. These results were then used to construct a predictive model of FTD dose as a function of % DMA with JMP® software (version 18.0.1) illustrated in Table 3.

TABLE 3
Predictive dose based on viscosity in DMA

| | | | Viscosity | Trifluridine |
|---|---|---|---|---|
| Trifluridine | DMA | Aqueous | at 37° C. | Dose^{a} |
| (mg/mL) | (% v/v) | Phase | (cP) | (mg/day) |
| 0 | 0 | 50 mM | 0.748 | 0 |
| 40 | 0 | citrate, 1000 | 0.827 | 43.4 |
| 0 | 10 | IU/mL heparin | 0.989 | 0 |
| 40 | 10 | sodium, pH 3.0 | 1.038 | 34.6 |
| 80 | 10 | | 1.135 | 63.3 |
| 0 | 30 | | 1.64 | 0 |
| 25 | 30 | | 1.756 | 12.8 |
| 50 | 30 | | 1.784 | 25.2 |
| 80 | 30 | | 1.975 | 36.4 |
| 100 | 30 | | 2.038 | 44 |
| 120 | 30 | | 2.14 | 50.3 |

^{a}Trifluridine daily doses were calculated using the 1.2 ml/day flow rate of a floxuridine drug solution which includes dexamethasone and heparin with a known viscosity of 0.839 cP, used for HAI infusion via the INTERA ® 3000 pump.

Solubility of trifluridine was measured in a variety of vehicles, including full aqueous solutions, as well as co-solvent systems of increasing concentrations of DMA. In parallel, the dynamic viscosity of some of these solutions was measured using a rheometer, and the daily dose that could be delivered for these trifluridine drug solutions using the INTERA® 3000 HAI pump were calculated using the known 1.2 ml/day daily volume of floxuridine solution (measured viscosity of 0.839 cP) that the pump delivers as a benchmark.

Daily flow of each sample using the INTERA® 3000 HAI pump was calculated based on the following formula:

$$Q = \frac{π {Pr}^{4}}{8 η l}$$

### Definition of Terms Used

Q=flow
P=pressure drop along the tube
d=diameter of the tube
r=radius of the tube
η=viscosity of the fluid
l=length of the tube

The following samples were prepared and viscosity was analyzed by a rheometer at 37° C. The results of these experiments are presented in Table 4.
1. 0% DMA, 0 mg/mL FTD, 0 mM citric acid
2. 0% DMA, 6.7 mg/ml floxuridine, 1,000 IU/ml heparin, 0.83 mg/ml dexamethasone
3. 0% DMA, 0 mg/ml FTD, 75 mM citric acid
4. 10% DMA, 0 mg/ml FTD, 75 mM citric acid
5. 15% DMA, 0 mg/ml FTD, 75 mM citric acid
6. 20% DMA, 0 mg/ml FTD, 75 mM citric acid
7. 0% DMA, 40 mg/ml FTD, 75 mM citric acid
8. 10% DMA, 40 mg/ml FTD, 75 mM citric acid
9. 15% DMA, 40 mg/ml FTD, 75 mM citric acid
10. 20% DMA, 40 mg/ml FTD, 75 mM citric acid
11. 10% DMA, 70 mg/ml FTD, 75 mM citric acid
12. 15% DMA, 76 mg/ml FTD, 75 mM citric acid
13. 20% DMA, 81 mg/ml FTD, 75 mM citric acid

TABLE 4
Viscosity analysis of solutions

| | Trifluridine | Additional | Viscosity at | Trifluridine |
|---|---|---|---|---|
| % DMA | concentration | vehicle | 37° C. | Dose |
| (v/v) | (mg/ml) | components | (cP) | (mg/day) |
| 0 | 0 | Water | 0.691 | 0 |
| 0 | 6.7 mg/ml | Saline, 1,000 | 0.839 | — |
| | Floxuridine | U/ml heparin, | | |
| | | 0.83 mg/ml | | |
| | | dexamethasone | | |
| 0 | 0 | 75 mM citric | 0.702 | 0 |
| 10 | 0 | acid | 0.935 | 0 |
| 15 | 0 | | 1.048 | 0 |
| 20 | 0 | | 1.221 | 0 |
| 0 | 40 | | 0.780 | 51.1 |
| 10 | 40 | | 1.003 | 39.8 |
| 15 | 40 | | 1.163 | 34.3 |
| 20 | 40 | | 1.373 | 29.1 |
| 10 | 70 | | 1.033 | 67.6 |
| 15 | 76 | | 1.205 | 62.9 |
| 20 | 81 | | 1.410 | 57.3 |

### Example 10

The effect of a co-solvent (DMA) on trifluridine solubility and formulation viscosity was determined based on the following protocol.

The following systems prepared based on the protocol described in Example 9 were evaluated.
1. 10% DMA in 75 mM citric acid, pH 3.1±0.1
2. 15% DMA in 75 mM citric acid, pH 3.1±0.1
3. 20% DMA in 75 mM citric acid, pH 3.1±0.1

To determine sampi solubility, the drugs were weighed into a filtration system to achieve a target concentration of 175 mg/mi trifluridine (FTD). The corresponding volume of dimethylacetamide (DMA) buffer was added to achieve 175 mg/ml trifluridine. The sample was vortexed and mixed for 72 hours at the designated temperature. The appearance was checked regularly and additional drug was added, if needed. The temperature was maintained and the sample was allowed to stand 1 to 2 hours, filtered, and diluted for HPLC analysis. The pH of filtrate was tested after returning to room temperature. The diluted samples were analyzed by HPLC against standards of known concentration. The following samples using the systems described above are shown below.
1. 20% DMA, temperature 40° C.
2. 10% DMA, temperature 27° C.
3. 15% DMA, temperature 5.2° C.
4. 10% DMA, temperature 40° C.
5. 10% DMA, temperature 5.2° C.
6. 15% DMA, temperature 27° C.
7. 15% DMA, temperature 40° C.
8. 20% DMA, temperature 5.2° C.
9. 15% DMA, temperature 27° C.
10. 20% DMA, temperature 27° C.

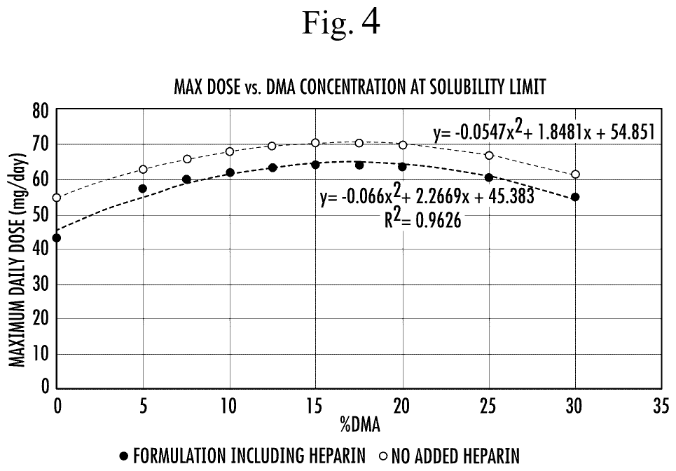

Solubility of FTD in varying amounts of DMA at multiple temperatures are presented in Table 5. These results were then used to construct a predictive model of trifluridine dose as a function of % DMA with JMP® software (version 18.0.1) (Table 6, FIG. 4).

TABLE 5
Solubility of FTD in varying amounts
of DMA at multiple temperatures

| | Aqueous | Temp | Solubility |
|---|---|---|---|
| % DMA | Phase | (° C.) | (mg/mL) |
| 10 | 75 mM citrate, | 5.2 | 73.44 |
| 10 | pH 3.0 | 27 | 74.34 |
| 10 | | 40 | 75.02 |
| 15 | | 5.2 | 76.91 |
| 15 | | 27 | 82.46 |
| 15 | | 27 | 82.75 |
| 15 | | 40 | 86.53 |
| 20 | | 5.2 | 85.35 |
| 20 | | 27 | 91.77 |
| 20 | | 40 | 113.46 |

TABLE 6
Predictive daily dose as a function of DMA concentration

| | Solubility | Daily dose |
|---|---|---|
| | (mg/ml, | (mg/day, |
| % DMA | at 5° C.) | at 5° C.) |
| 10 | 72.8 | 71.2 |
| 11 | 73.7 | 70.0 |
| 12 | 74.6 | 68.9 |
| 13 | 75.5 | 67.6 |
| 14 | 76.4 | 66.3 |
| 16 | 78.3 | 63.6 |
| 18 | 80.1 | 60.7 |
| 20 | 81.9 | 57.6 |

### Example 11

The anticoagulant effect of citrate buffer compared to heparin and fondaparinux was determined based on the following protocol.

Rat citrated plasma was collected from Sprague Dawley (SD) rats. Plasma from SD rats was obtained by centrifugation. Lyophilized human plasma was dissolved with 1 ml of sterile water for injection. The plasma was kept at 15-25° C. for 30 minutes and inverted to mix before use. A 1,000 IU heparin solution and a 0.835 mg/ml fondaparinux (FPX) solution were prepared using sterile water for injection (Table 4). Citrate buffer solutions were prepared at concentration of 0 mM, 100 mM, 125 mM, 240 mM, 250 mM, 375 mM, 500 mM, 625 mM, 750 mM, 875 mM, 1000 mM, and 1125 mM using sterile water for injection. Plasma was mixed with anticoagulant and activated partial thromboplastin time (APTT) reagents per manufacturer's instructions. The time to coagulation (in seconds) was measured using a coagulation analyzer. Citric acid solution with similar time-to-coagulation compared to 1,000 IU/ml of heparin and 0.167 mg/ml of fondaparinux were identified (Tables 7-9).

TABLE 7
Anticoagulant heparin and citrate results in rat.

| | Rat - aPTT |
|---|---|
| Anticoagulant | results (sec) |
| 1,000 U/ml heparin | 13.1 |
| 100 mM citrate | No results, Clot |
| 125 mM citrate | No results, Clot |
| 250 mM citrate | 18.4, followed by small clots |
| 500 mM citrate | No results, no clots |
| 625 mM citrate | No results, no clots |

TABLE 8
Anticoagulant fondaparinux and citrate in rat.

| | Rat - aPTT |
|---|---|
| Anticoagulant | results (sec) |
| 0.167 mg/ml FPX | 147.6 |
| 0 mM citrate | 22 |
| 48 mM citrate | 139 |
| 50 mM citrate | 167.3 |
| 75 mM citrate | Undetectable |
| 100 mM citrate | Undetectable |
| 125 mM citrate | Undetectable |

TABLE 9
Anticoagulant fondaparinux and citrate in human.

| | Human - aPTT |
|---|---|
| Anticoagulant | results (sec) |
| 0.167 mg/ml FPX | 75.6 |
| 0 mM citrate | 27.2 |
| 34 mM citrate | 64.9 |
| 35 mM citrate | 76.4 |
| 36 mM citrate | 86.0 |
| 40 mM citrate | Undetectable |

It is to be appreciated that the Detailed Description section, and not the Summary and Abstract sections, is intended to be used to interpret the claims. The Summary and Abstract sections may set forth one or more but not all exemplary embodiments of the present invention as contemplated by the inventor(s), and thus, are not intended to limit the present invention and the appended claims in any way.

The present invention has been described above with the aid of functional building blocks illustrating the implementation of specified functions and relationships thereof. The boundaries of these functional building blocks have been arbitrarily defined herein for the convenience of the description. Alternate boundaries can be defined so long as the specified functions and relationships thereof are appropriately performed.

The foregoing description of the specific embodiments will so fully reveal the general nature of the invention that others can, by applying knowledge within the skill of the art, readily modify and/or adapt for various applications such specific embodiments, without undue experimentation, without departing from the general concept of the present invention. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

The breadth and scope of the present invention should not be limited by any of the above-described exemplary embodiments, but should be defined only in accordance with the following claims and their equivalents.

What is claimed is:

1. A liquid pharmaceutical formulation comprising:
about 50 mg/ml to about 150 mg/ml of trifluridine,
about 5 mM to about 250 mM of buffer, and
a solvent system comprising water and a co-solvent,
wherein the formulation has a pH of about 1 to 8.

2. The formulation of claim 1, wherein the formulation comprises about 50 mg/ml to about 100 mg/ml of trifluridine.

3. The formulation of claim 1, wherein the formulation comprises about 25 mM to about 100 mM of buffer.

4. The formulation of claim 1, wherein the solvent system comprises about 10% to about 30% v/v of co-solvent.

5. The formulation of claim 1, wherein the pH is about 2 to about 5.

6. A method of treating liver cancer in a subject in need thereof, the method comprising administering to the subject a therapeutically effective amount of the formulation of claim 1 by hepatic arterial infusion.

7. A liquid pharmaceutical formulation comprising:
about 50 mg/ml to about 100 mg/ml of trifluridine,
about 25 mM to about 100 mM of citrate buffer,
a solvent system comprising water and about 10% to about 30% v/v dimethylacetamide,
wherein the formulation has a pH of about 2 to 5.

8. A method of treating liver cancer in a subject in need thereof, the method comprising administering to the subject a therapeutically effective amount of trifluridine by hepatic arterial infusion.

9. The method of claim 1, wherein the therapeutically effective amount of trifluridine is administered via a pump.

10. The method of claim 8, wherein the therapeutically effective amount of trifluridine is about 0.01 mg/kg/day to about 3 mg/kg/day.

11. The method of claim 8, wherein the therapeutically effective amount of trifluridine is administered for at least 7 days.

12. The method of claim 8, wherein, after the therapeutically effective amount of trifluridine is administered for at least 7 days, the subject's alanine aminotransferase level does not rise more than about 20% above the subject's alanine aminotransferase level prior to administration of the therapeutically effective amount of trifluridine.

13. The method of claim 8, wherein, after the therapeutically effective amount of trifluridine is administered for at least 7 days, the subject's alanine aminotransferase level does not rise more than about 20 U/L above the subject's alanine aminotransferase level prior to administration of the therapeutically effective amount of trifluridine.

14. The method of claim 8, wherein, after the therapeutically effective amount of trifluridine is administered for at least 7 days, the subject's aspartate aminotransferase level does not rise more than about 20% above the subject's aspartate aminotransferase level prior to administration of the therapeutically effective amount of trifluridine.

15. The method of claim 8, wherein, after the therapeutically effective amount of trifluridine is administered for at least 7 days, the subject's aspartate aminotransferase level does not rise more than about 20 U/L above the subject's aspartate aminotransferase level prior to administration of the therapeutically effective amount of trifluridine.

16. The method of claim 8, wherein, after the therapeutically effective amount of trifluridine is administered for at least 7 days, the subject's alkaline phosphatase level does not rise more than about 20% above the subject's alkaline phosphatase level prior to administration of the therapeutically effective amount of trifluridine.

17. The method of claim 8, wherein, after the therapeutically effective amount of trifluridine is administered for at least 7 days, the subject's alkaline phosphatase level does not rise more than about 50 IU/L above the subject's alkaline phosphatase level prior to administration of the therapeutically effective amount of trifluridine.

18. The method of claim 8, wherein, after the therapeutically effective amount of trifluridine is administered for at least 7 days, the subject's total bilirubin level does not rise more than about 20% above the subject's total bilirubin level prior to administration of the therapeutically effective amount of trifluridine.

19. The method of claim 8, wherein, after the therapeutically effective amount of trifluridine is administered for at least 7 days, the subject's total bilirubin level does not rise more than about 1 mg/dL above the subject's total bilirubin level prior to administration of the therapeutically effective amount of trifluridine.

20. The method of claim 8, wherein the subject has a plasma concentration of trifluridine of less than about 0.1 μM after the therapeutically effective amount of trifluridine is administered for at least 7 days.
